(19) United States
(12) Patent Application Publication
(10) Pub. No.: US 2023/0116809 A1
(43) Pub. Date: Apr. 13, 2023

(54) PREVENTION AND TREATMENT OF OCULAR CONDITIONS

(71) Applicant: Ascendis Pharma Ophthalmology Division A/S, Hellerup (DK)

(72) Inventors: Thomas KNAPPE, Heidelberg (DE); Burkhardt Laufer, Dossenheim (DE); Harald Rau, Dossenheim (DE); Kennett Sprogøe, Holte (DK); Tobias Voigt, Heidelberg (DE); Samuel Weisbrod, Heidelberg (DE)

(21) Appl. No.: 17/937,564

(22) Filed: Oct. 3, 2022

Related U.S. Application Data
(63) Continuation of application No. 15/400,887, filed on Jan. 6, 2017, now abandoned.
Application No. 15/400,887 is a division of application No. 14/350,394, filed on Apr. 8, 2014, now abandoned.

(30) Foreign Application Priority Data
Oct. 12, 2011 (EP) 11184865.1

(51) Int. Cl. A61K 47/60; A61K 9/00; A61K 31/573; A61K 47/69

(52) U.S. Cl. CPC A61K 47/60; A61K 9/0048; A61K 31/573; A61K 47/6903; A61K 47/26

(57) ABSTRACT

The present invention relates to pharmaceutical compositions comprising hydrogel-linked prodrug for use in the treatment, prevention and/or diagnosis a condition of the eye and ophthalmic devices comprising said pharmaceutical compositions.

The present application is a divisional of U.S. patent application Ser. No. 14/350,394 filed on Apr. 8, 2014, which claims priority from PCT Patent Application No. PCT/EP2012/070212 filed on Oct. 11, 2012, which claims priority from European Patent Application No. EP 11184865.1 filed on Oct. 12, 2011, the disclosures of which are incorporated herein by reference in their entirety.

### FIELD OF THE INVENTION

A leading cause of blindness is the inability to introduce drugs or therapeutic agents into the eye and maintain these drugs or agents at a therapeutically effective concentration therein for the necessary duration. Systemic administration may not be an ideal solution because, often, unacceptably high levels of systemic dosing is needed to achieve effective intraocular concentrations, with the increased incidence of unacceptable side effects of the drugs. Simple ocular instillation or application is not an acceptable alternative in many cases because the drug may be quickly washed out by tear-action or is depleted from within the eye into the general circulation.

Thus, there is widespread recognition in the field of ophthalmology that controlled release drug delivery systems would benefit patient care and ocular health by providing extended delivery of therapeutic agents to the eye while minimizing the problems associated with patient compliance to prescribed therapeutic medical regimens. Although a wide variety of drug delivery methods exist, topical eye drop therapy is limited by poor absorption, a need for frequent and/or chronic dosing over periods of days to years, rapid turnover of aqueous humor, production and movement of the tear film and other causes, which may effectively remove therapeutic agents long before therapy has been completed or the proper dose delivered.

A solution to this problem would be to provide a delivery device which can be implanted into the eye such that a controlled amount of desired drug can be released constantly over a period of several days, or weeks, or even months. Some such devices have been reported in the prior art. See, for example, U.S. Pat. No. 4,853,224, which discloses biocompatible implants for introduction into an anterior segment or posterior segment of an eye for the treatment of an ocular condition. U.S. Pat. No. 5,164,188 discloses a method of treating an ocular condition by introduction of a biodegradable implant comprising drugs of interest into the suprachoroidal space or pars plana of the eye. See also U.S. Pat. Nos. 5,824,072, 5,476,511, 4,997,652, 4,959,217, 4,668,506, and 4,144,317. However, it is desirable to avoid surgery of the eye, so implants are not necessarily the ideal tool for drug delivery.

Intravitreal injections are commonly used to deliver therapeutic agents to the eye, particularly to the vitreous humor of the eye for treatment of ophthalmic maladies such as age related macular degeneration (AMD), diabetic macular edema (DME), inflammation or the like. Intravitreal injections are often particularly desirable since they can provide enhanced bioavailability to a target location (e.g., the retina) of the eye relative to other delivery mechanisms such as topical delivery.

It is noted that citation or identification of any document in this application is not an admission that such document is available as prior art to the present invention.

While generally providing a desirable form of drug delivery, intravitreal injections also have drawbacks and can present various different complications. For example, intravitreal injections can result in delivery of undesirably high concentrations of therapeutic agent to a target location or elsewhere particularly when the therapeutic agent is relatively soluble.

In addition to the above, therapeutic agents delivered by intravitreal injections can lack duration of action since the agents can often rapidly disperse within the eye after injection. Such lack of duration is particularly undesirable since it can necessitate greater injection frequency.

In view of the above, there exists a need to provide a form of administration that overcomes these drawbacks at least partially.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right to disclaim, and hereby disclose a disclaimer of, any previously described product, method of making the product, or process of using the product.

### SUMMARY OF THE INVENTION

This objective is achieved with a hydrogel-linked prodrug and/or a pharmaceutical composition comprising a hydrogel-linked prodrug for use in the prevention, diagnosis and/or treatment of an ocular condition.

Preferred is the prevention and/or treatment of an ocular condition.

The invention also relates to a method of preventing and/or treating an ocular disease, wherein said method comprises the step of administering a therapeutically effective amount of a hydrogel-linked-prodrug or pharmaceutical composition of the present invention to a patient in need thereof.

In another embodiment this invention relates to a hydrogel-linked prodrug and/or a pharmaceutical composition comprising a hydrogel-linked prodrug for use for intraocular injection. Preferably, the intraocular injection is an intravitreal injection into the vitreous body.

In a further embodiment the present invention relates to a hydrogel-linked prodrug and/or a pharmaceutical composition comprising a hydrogel-linked prodrug for use for intraocular injection in the prevention, diagnosis and/or treatment of an ocular condition.

Preferably, the intraocular injection is an intravitreal injection into the vitreous body.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

It was now surprisingly found that hydrogel-linked prodrugs provide a long-lasting depot which is beneficial for the prevention, diagnosis and/or treatment of an ocular condition. Such hydrogel-linked prodrugs are carrier-linked prodrugs in which the carrier is a hydrogel and to which biologically active moieties are connected through reversible prodrug linkers and which biologically active moieties are released from the carrier-linked prodrug in the form of a drug.

As the drug is released in therapeutically effective concentrations over an extended period of time, overconcentration of the drug is avoided. A single intraocular injection is also less invasive than the surgical procedures needed for ophthalmic implants.

Within the present invention the terms are used having the meaning as follows.

As used herein, an “ocular condition” is a disease, ailment or condition which affects or involves the eye or one of the parts or regions of the eye. Broadly speaking, the eye includes the eyeball and the tissues and fluids which constitute the eyeball, the periocular muscles (such as the oblique and rectus muscles) and the portion of the optic nerve which is within or adjacent to the eyeball.

The terms “drug”, “biologically active molecule”, “biologically active moiety”, “biologically active agent”, “active agent”, “active substance” and the like mean any substance which can affect any physical or biochemical properties of a biological organism, including but not limited to viruses, bacteria, fungi, plants, animals, and humans. In particular, as used herein, the terms include any substance intended for diagnosis, cure, mitigation, treatment, or prevention of disease in organisms, in particular humans or other animals, or to otherwise enhance physical or mental well-being of organisms, in particular humans or animals.

“Biologically active moiety D” means the part of a biologically active moiety-reversible prodrug linker conjugate or the part of a biologically active moiety-reversible prodrug linker-carrier conjugate, which results after cleavage in a drug D-H of known biological activity. In particular, the drug D-H is suitable for treating, diagnosing and/or preventing at least one condition of the eye in at least one organism, in particular humans. According to the present invention, the biologically active moiety-reversible prodrug linker-carrier conjugate is a hydrogel-linked prodrug.

“Amine-containing biologically active moiety” or “hydroxyl-containing biologically active moiety” means the part (moiety or fragment) of a biologically active moiety-reversible prodrug linker conjugate or the part of a biologically active moiety-reversible prodrug linker-carrier conjugate (active agent) of (known) biological activity, and which part of the drug comprises at least one amine or hydroxyl group, respectively.

Accordingly, as used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a biologically active moiety is released from a prodrug as a drug.

In addition, the subterm “aromatic amine-containing” means that the respective biologically active moiety D and analogously the corresponding drug D-H contains at least one aromatic fragment which is substituted with at least one amino group. The subterm “aliphatic amine-containing” means that the respective biologically active moiety D and analogously the corresponding drug D-H contains at least one aliphatic fragment which is substituted with at least one amino group. Without further specification the term “amine-containing” is used generically and refers to aliphatic and aromatic amine-containing moieties.

The subterm “aromatic hydroxyl-containing” means that the respective moiety D and analogously the corresponding drug D-H contains at least one aromatic fragment, which is substituted with at least one hydroxyl group. The subterm “aliphatic hydroxyl-containing” means that the hydroxyl group of the respective moiety D and analogously the corresponding drug D-H is connected to an aliphatic fragment. Without further specification the term “hydroxyl-containing” is used generically and refers to aliphatic and aromatic hydroxyl-containing moieties.

“Pharmaceutical composition” or “composition” means a composition containing one or more prodrugs, and optionally one or more excipients, as well as any product which results, directly or indirectly, from combination, complexation or aggregation of any of the excipients and/or prodrug(s), or from dissociation of any of the excipients and/or prodrug(s), or from other types of reactions or interactions of any of the excipients and/or prodrug(s). Accordingly, a pharmaceutical composition of the present invention encompasses any composition obtainable by admixing a hydrogel-linked prodrug of the present invention and a pharmaceutically acceptable excipient.

The term “excipient” refers to a diluent, adjuvant, or vehicle with which the hydrogel-linked prodrug is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These compositions can take the form of solutions, suspensions, emulsions, tablets, pills, capsules, powders, sustained-release formulations and the like.

The composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a diagnostically and/or therapeutically effective amount of the a hydrogel-linked prodrug, preferably in purified form, together with a suitable amount of excipient so as to provide the form for proper administration to the patient. The formulation should suit the mode of administration.

The term “intraocular injection” refers to an injection into the aqueous humor (anterior or posterior chamber), the vitreous body or lens.

To enhance physicochemical or pharmacokinetic properties of a drug in vivo, such drug can be conjugated with a carrier. If the drug is transiently bound to a carrier and/or a linker, as in the hydrogel-linked prodrug comprised in the pharmaceutical composition for use in the prevention, diagnosis and/or treatment of an ocular condition of the present invention, such systems are commonly assigned as “carrier-linked prodrugs”. According to the definitions provided by IUPAC (as given under http://www.chem.qmul.ac.uk/iupac/medchem/ah.html, accessed on Mar. 7, 2011), a carrier-linked prodrug is a prodrug that contains a temporary linkage of a given active substance with a transient carrier group that produces improved physicochemical or pharmacokinetic properties and that can be easily removed in vivo, usually by a hydrolytic cleavage. In other words, a carrier-linked prodrug comprises three components, namely the biologically active moiety which is connected to a reversible prodrug linker moiety which reversible prodrug moiety is connected to a carrier. The linkage between the biologically active moiety and the reversible prodrug linker is reversible, whereas the linkage between the reversible prodrug linker and the carrier is preferably a stable linkage. It is understood that a hydrogel-linked prodrug is a carrier-linked prodrug in which the carrier is a hydrogel.

The term “promoiety” refers to the part of the prodrug which is not the drug, thus meaning linker and carrier and/or any optional spacer moieties.

The terms “hydrolytically degradable”, “biodegradable”, “auto-cleavable”, “self-cleavable”, “reversible” or “transient” refer to bonds and linkages which are non-enzymatically hydrolytically degradable or cleavable under physiological conditions (aqueous buffer at pH 7.4, 37° C.) with half-lives ranging from one hour to nine months, including, but are not limited to, aconityls, acetals, amides, carboxylic anhydrides, esters, imines, hydrazones, maleamic acid amides, ortho esters, phosphamides, phosphoesters, phosphosilyl esters, silyl esters, sulfonic esters, aromatic carbamates, carbamates, sulfonamides, N-acetylsulfonamides, thiocarbamates, and combinations thereof, and the like. Preferred bonds and linkages which are non-enzymatically hydrolytically degradable or cleavable under physiological conditions (aqueous buffer at pH 7.4, 37° C.) with half-lives ranging from one hour to nine months are selected from aconityls, acetals, amides, carboxylic anhydrides, esters, imines, hydrazones, maleamic acid amides, ortho esters, phosphamides, phosphoesters, phosphosilyl esters, silyl esters, sulfonic esters, aromatic carbamates, and combinations thereof. On the other hand, stable or permanent linkages are typically non-cleavable permanent bonds, meaning that they have a half-life of at least twelve months under physiological conditions (aqueous buffer at pH 7.4, 37° C.).

A “traceless prodrug linker” refers to a prodrug linker from which a drug is released in its free form, meaning that upon release from the promoiety the drug does not contain any traces of the promoiety.

“Free form” of a drug refers to the drug in its unmodified, pharmacologically active form, such as after being released from a traceless prodrug linker.

The term “hydrogel” refers to a three-dimensional, hydrophilic or amphiphilic polymeric network capable of taking up large quantities of water which causes swelling of the hydrogel in aqueous media. The networks are composed of homopolymers or copolymers and are insoluble due to the presence of covalent chemical or physical (ionic, hydrophobic interactions, entanglements) crosslinks. The crosslinks provide the network structure and physical integrity.

The term “polymer” describes a molecule comprising repeating structural units connected by chemical bonds in a linear, circular, branched, crosslinked or dendrimeric way or a combination thereof, which can be of synthetic or biological origin or a combination of both. Typically, a polymer has a molecular weight of at least 500 Da. It is understood, that when the polymer is a polypeptide, then the individual amino acids of the polypeptide may be the same or may be different.

The term “polymeric” refers to a moiety comprising at least one polymer.

It is understood that all reagents and moieties comprising one or more polymer(s) refer to macromolecular entities known to exhibit variations with respect to molecular weight, chain lengths or degree of polymerization, or the number of functional groups and chemical functional groups. Structures shown and molecular weights given for backbone reagents, backbone moieties, crosslinker reagents, crosslinker moieties or other moieties and reagents are thus only representative examples.

The term “poly(ethylene glycol) based polymeric chain” or “PEG based chain” refers to an oligo- or polymeric molecular chain comprising ethylene glycol monomers.

The term “PEG-based” as understood herein means that the mass proportion of PEG chains in the hydrogel according to the invention is at least 10% by weight, preferably at least 20% by weight, and even more preferably at least 25% by weight based on the total weight of the hydrogel according to the invention. The remainder can be made up of other polymers.

If the term “poly(ethylene glycol) based polymeric chain” is used in reference to a crosslinker reagent or to a crosslinker, it refers to a crosslinker moiety or chain comprising at least 20 weight % ethylene glycol moieties.

The phrases “in bound form”, “connected to”, and “moiety” refer to sub-structures which are part of a molecule. The phrases “in bound form” or “connected to” are used to simplify reference to moieties or functional groups or chemical functional groups by naming or listing reagents, starting materials or hypothetical starting materials well known in the art, and whereby “in bound form” and “connected to” means that for example one or more hydrogen radicals (—H) or one or more activating or protecting groups present in the reagents or starting materials are not present in the moiety when part of a molecule.

As used herein, the term “immiscible” means the property where two substances are not capable of combining to form a homogeneous mixture.

The term “chemical functional group” refers to carboxylic acid and activated derivatives, amino, maleimide, thiol and derivatives, sulfonic acid and derivatives, carbonate and derivatives, carbamate and derivatives, hydroxyl, aldehyde, ketone, hydrazine, isocyanate, isothiocyanate, phosphoric acid and derivatives, phosphonic acid and derivatives, haloacetyl, alkyl halides, acryloyl and other alpha-beta unsaturated michael acceptors, arylating agents like aryl fluorides, hydroxylamine, disulfides like pyridyl disulfide, vinyl sulfone, vinyl ketone, diazoalkanes, diazoacetyl compounds, oxirane, and aziridine.

If a chemical functional group is coupled to another chemical functional group or functional group, the resulting chemical structure is referred to as “linkage”. For example, the reaction of an amine group with a carboxyl group results in an amide linkage. The terms “linkage” and “bond” are used synonymously.

The term “interconnectable functional group” refers to chemical functional groups, which participate in a radical polymerization reaction and are part of the crosslinker reagent or the backbone reagent.

The term “polymerizable functional group” refers to chemical functional groups, which participate in a ligation-type polymerization reaction and are part of the crosslinker reagent and the backbone reagent.

“Reactive functional groups” are chemical functional groups of the backbone moiety, which are connected to the hyperbranched moiety.

“Functional group” is the collective term used for “reactive functional group”, “degradable interconnected functional group”, or “conjugate functional group”.

A “degradable interconnected functional group” is a linkage comprising a biodegradable bond which on one side is connected to a spacer moiety connected to a backbone moiety and on the other side is connected to the crosslinking moiety. The terms “degradable interconnected functional group”, “biodegradable interconnected functional group”, “interconnected biodegradable functional group” and “interconnected functional group” are used synonymously.

As used herein, the term “activated functional group” means a functional group, which is connected to an activating group, i.e. a functional group was reacted with an activating reagent. Preferred activated functional groups include but are not limited to activated ester groups, activated carbamate groups, activated carbonate groups and activated thiocarbonate groups. Preferred activating groups are selected from formulas ((f-i) to (f-vi):

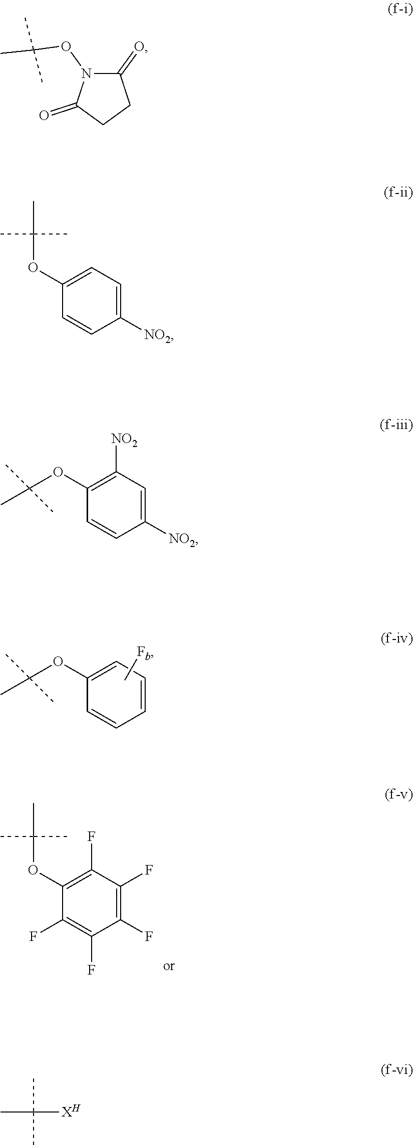

wherein
the dashed lines indicate attachment to the rest of the molecule;
b is 1, 2, 3 or 4; and
X^{H} is Cl, Br, I, or F.

Accordingly, a preferred activated ester has the formula

—(C═O)—X^{F},
wherein
X^{F} is selected from formula (f-i), (f-ii), (f-iii), (f-iv), (f-v) and (f-vi).

Accordingly, a preferred activated carbamate has the formula

—N—(C═O)—X^{F},
wherein
X^{F} is selected from formula (f-i), (f-ii), (f-iii), (f-iv), (f-v) and (f-vi).

Accordingly, a preferred activated carbonate has the formula

—O—(C═O)—X^{F},
wherein
X^{F} is selected from formula (f-i), (f-ii), (f-iii), (f-iv), (f-v) and (f-vi).

Accordingly, a preferred activated thioester has the formula

—S—(C═O)—X^{F},
wherein
X^{F} is selected from formula (f-i), (f-ii), (f-iii), (f-iv), (f-v) and (f-vi).

Accordingly, an “activated end functional group” is an activated functional group which is localized at the end of a moiety or molecule, i.e. is a terminal activated functional group.

The terms “blocking group” or “capping group” are used synonymously and refer to moieties which are irreversibly (especially permanent) connected to reactive functional groups or chemical functional groups to render them incapable of reacting with for example chemical functional groups.

The terms “protecting group” or “protective group” refers to a moiety which is reversibly connected to reactive functional groups or chemical functional groups to render them incapable of reacting with for example other chemical functional groups.

The term “reagent” refers to an intermediate or starting reagent used in the assembly process leading to hydrogels, conjugates, and prodrugs.

“Alkyl” means a straight-chain, branched or cyclic carbon chain (unsubstituted alkyl). Optionally, one or more hydrogen atoms of an alkyl carbon may be replaced by a substituent. In general, a preferred alkyl is C₁₋₆ alkyl.

“C₁₋₄ alkyl” means an alkyl chain having 1 to 4 carbon atoms (unsubstituted C₁₋₄ alkyl), e.g. if present at the end of a molecule: methyl, ethyl, n-propyl, isopropyl, n-butyl, isobutyl, sec-butyl tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group (also referred to as C₁₋₄ alkylene). Optionally, one or more hydrogen atom(s) of a C₁₋₄ alkyl carbon may be replaced by a substituent as indicated herein. Accordingly, “C₁₋₅₀ alkyl” means an alkyl chain having 1 to 50 carbon atoms.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl, tert-butyl, n-pentyl, n-hexyl, or e.g. —CH₂—, —CH₂—CH₂—, —CH(CH₃)—, —C(CH₂)—, —CH₂—CH₂—CH₂—, —CH(C₂H₅)—, —C(CH₃)₂—, when two moieties of a molecule are linked by the alkyl group (also referred to as C₁₋₆ alkylene). One or more hydrogen atom(s) of a C₁₋₆ alkyl carbon may be replaced by a substituent as indicated herein. The terms C₁₋₁₅ alkyl or C₁₋₁₅ alkylene are defined accordingly.

“C₂₋₆ alkenyl” means an alkenyl chain having 2 to 6 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. One or more hydrogen atom(s) of a C₂₋₆ alkenyl carbon may be replaced by a substituent as indicated herein.

The term C₂₋₄ alkenyl is defined accordingly.

“C₂₋₆ alkynyl” means an alkynyl chain having 2 to 6 carbon atoms, e.g. if present at the end of a molecule: —C≡CH, —CH₂—C≡CH, CH₂—CH₂—C≡CH, CH₂—C≡C≡CH₃, or e.g. —C≡C— when two moieties of a molecule are linked by the alkynyl group. One or more hydrogen atom(s) of a C₂₋₆ alkynyl carbon may be replaced by a substituent as indicated herein. The term C₂₋₄ alkynyl is defined accordingly.

“C₂₋₅₀ alkenyl” means a branched, unbranched or cyclic alkenyl chain having 2 to 50 carbon atoms (unsubstituted C₂₋₅₀ alkenyl), e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group.

Optionally, one or more hydrogen atom(s) of a C₂₋₅₀ alkenyl carbon may be replaced by a substituent as further specified. Accordingly, the term “alkenyl” relates to a carbon chain with at least one carbon carbon double bond. Optionally, one or more triple bonds may occur. The term “C₂₋₁₅ alkenyl” is defined accordingly.

“C₂₋₅₀ alkynyl” means a branched, unbranched or cyclic alkynyl chain having 2 to 50 carbon atoms (unsubstituted C₂₋₅₀ alkynyl), e.g. if present at the end of a molecule: —C≡CH, —CH₂—C≡CH, CH₂—CH₂—C≡CH, CH₂—C≡C≡CH₃, or e.g. —C≡C— when two moieties of a molecule are linked by the alkynyl group. Optionally, one or more hydrogen atom(s) of a C₂₋₅₀ alkynyl carbon may be replaced by a substituent as further specified. Accordingly, the term “alkynyl” relates to a carbon chain with at least one carbon triple bond. Optionally, one or more double bonds may occur.

“C₃₋₇ cycloalkyl” or “C₃₋₇ cycloalkyl ring” means a cyclic alkyl chain having 3 to 7 carbon atoms, which may have carbon-carbon double bonds being at least partially saturated (unsubstituted C₃₋₇ cycloalkyl), e.g. cyclopropyl, cyclobutyl, cyclopentyl, cyclohexyl, cyclohexenyl, cycloheptyl. Optionally, one or more hydrogen atom(s) of a cycloalkyl carbon may be replaced by a substituent as indicated herein. The term “C₃₋₇ cycloalkyl” or “C₃₋₇ cycloalkyl ring” also includes bridged bicycles like norbonane (norbonanyl) or norbonene (norbonenyl). Accordingly, “C₃₋₅ cycloalkyl” means a cycloalkyl having 3 to 5 carbon atoms. Accordingly, “C₃₋₈ cycloalkyl” means a cycloalkyl having 3 to 8 carbon atoms. Accordingly, “C₃₋₁₀ cycloalkyl” means a cycloalkyl having 3 to 10 carbon atoms.

“Halogen” means fluoro, chloro, bromo or iodo. It is generally preferred that halogen is fluoro or chloro.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom (unsubstituted 4 to 7 membered heterocyclyl). For the sake of completeness it is indicated that in some embodiments of the present invention, 4 to 7 membered heterocyclyl has to fulfill additional requirements. Examples for a 4 to 7 membered heterocycles are azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine. Optionally, one or more hydrogen atom(s) of a 4 to 7 membered heterocyclyl may be replaced by a substituent.

“8 to 11 membered heterobicyclyl” or “8 to 11 membered heterobicycle” means a heterocyclic system of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom (unsubstituted 8 to 11 membered heterobicyclyl). Examples for a 8 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine or pteridine. The term 8 to 11 membered heterobicycle also includes spiro structures of two rings like 1,4-dioxa-8-azaspiro[4.5]decane or bridged heterocycles like 8-aza-bicyclo[3.2.1]octane. The term “9 to 11 membered heterobicyclyl” or “9 to 11 membered heterobicycle” is defined accordingly.

The term “aliphatic” means a fully saturated or unsaturated hydrocarbon, such as an alkyl, alkenyl or alkynyl.

As used herein, the term “polyamine” means a reagent or moiety comprising more than one amine (—NH— and/or —NH₂), e.g. from 2 to 64 amines, from 4 to 48 amines, from 6 to 32 amines, from 8 to 24 amines, or from 10 to 16 amines. Particularly preferred polyamines comprise from 2 to 32 amines.

The term “derivatives” refers to chemical functional groups or functional groups suitably substituted with protecting and/or activation groups or to activated forms of a corresponding chemical functional group or functional group which are known to the person skilled in the art. For example, activated forms of carboxyl groups include but are not limited to active esters, such as succinimidyl ester, benzotriazyl ester, nitrophenyl ester, pentafluorophenyl ester, azabenzotriazyl ester, acyl halogenides, mixed or symmetrical anhydrides, acyl imidazole.

In general the term “substituted” preferably refers to substituents, which are the same or different and which are independently selected from the group consisting of halogen, CN, COOR^{b9}, OR^{b9}, C(O)R^{b9}, C(O)N(R^{b9}R^{b9a}), S(O)₂N(R^{b9}R^{b9a}), S(O)N(R^{b9}R^{b9a}), S(O)₂R^{b9}, S(O)R^{b9}, N(R^{b9})S(O)₂N(R^{b9a}R^{b9b}), SR^{b9}, N(R^{b9}R^{b9a}), NO₂, OC(O)R^{b9}, N(R^{b9})C(O)R^{b9a}, N(R^{b9})S(O)₂R^{b9a}, N(R^{b9})S(O)R^{b9a}, N(R^{b9})C(O)OR^{b9a}, N(R^{b9})C(O)N(R^{b9a}R^{b9b)}, OC(O)N(R^{b9}R^{9a}), T^{b}, C₁₋₅₀ alkyl, C₂₋₅₀ alkenyl, and C₂₋₅₀ alkynyl,
wherein T^{b}, C₁₋₅₀ alkyl, C₂₋₅₀ alkenyl, and C₂₋₅₀ alkynyl are optionally substituted with one or more R^{b10}, which are the same or different, and wherein C₁₋₅₀ alkyl; C₂₋₅₀ alkenyl; and C₂₋₅₀ alkynyl are optionally interrupted by one or more groups selected from the group consisting of T^{b}, —C(O)O—; —O—; —C(O)—; —C(O)N(R^{b11})—; —S(O)₂N(R^{b11})—; —S(O)N(R^{b11})—; —S(O)₂—; —S(O)—; —N(R^{b11})S(O)₂N(R^{b11a})—; —N(R^{b11})—; —OC(O)R^{b11}; —N(R^{b11})C(O)—; —N(R^{b11})S(O)₂—; —N(R^{b11})S(O)—; —N(R^{b11})C(O)O—; —N(R^{b11})C(O)N(R^{b11a}); and —OC(O)N(R^{b11}R^{b11a});
R^{b9}, R^{b9a}, R^{b9b} are independently selected from the group consisting of H; T^{b}; and C₁₋₅₀ alkyl; C₂₋₅₀ alkenyl; and C₂₋₅₀ alkynyl,
   wherein T^{b}, C₁₋₅₀ alkyl, C₂₋₅₀ alkenyl, and C₂₋₅₀ alkynyl are optionally substituted with one or more R^{b10}, which are the same or different, and wherein C₁₋₅₀ alkyl; C₂₋₅₀ alkenyl; and C₂₋₅₀ alkynyl are optionally interrupted by one or more groups selected from the group consisting of T^{b}, —C(O)O—, —O—, —C(O)—, —C(O)N(R^{b11})—, —S(O)₂N(R^{b11})—, —S(O)N(R^{b11})—, —S(O)₂—, —S(O)—, —N(R^{b11})S(O)₂N(R^{b11a})—, —S—, —N(R^{b11})—, —OC(O)R^{b11}, —N(R^{b11})C(O)—, —N(R^{b11})S(O)₂—, —N(R^{b11})S(O)—, —N(R^{b11})C(O)O—, —N(R^{b11})C(O)N(R^{b11a})—, and —OC(O)N(R^{b11}R^{b11a}),
   T^{b} is selected from the group consisting of phenyl, naphthyl, indenyl, indanyl, tetralinyl, C₃₋₁₀ cycloalkyl, 4- to 7-membered heterocyclyl, and 9- to 11-membered heterobicyclyl, wherein T^{b} is optionally substituted with one or more R^{b10}, which are the same or different,
   R^{b10} is halogen, CN, oxo (═O), COOR^{b12}, OR^{b12}, C(O)R^{b12}, —C(O)N(R^{b12}R^{b12a}), S(O)₂N(R^{b12}R^{b12a}), S(O)N(R^{b12}R^{b12a}), S(O)₂R^{b12}, S(O)R^{b12}, N(R^{b12})S(O)₂N(R^{b12a}R^{b12}), SR^{b12}, N(R^{b12}R^{b12a}), NO₂, OC(O)R^{b12}, N(R^{b12})C(O)R^{b12a}, N(R^{b12})S(O)₂R^{b12a}, N(R^{b12})S(O)R^{b12a}, N(R^{b12})C(O)OR^{b12a}, N(R^{b12})C(O)N(R^{12a}R^{b12b}), OC(O)N(R¹²R^{b12a}), or C₁₋₆ alkyl, wherein C₁₋₆ alkyl is optionally substituted with one or more halogen, which are the same or different,
   R^{b11}, R^{b11a}, R^{b12}, R^{b12a}, R^{b12b} are independently selected from the group consisting of H; or C₁₋₆ alkyl, wherein C₁₋₆ alkyl is optionally substituted with one or more halogen, which are the same or different.

Preferably, R⁹, R^{9a}, R^{9b} may be independently of each other H.

Preferably, R¹⁰ is C₁₋₆ alkyl.

Preferably, T is phenyl.

Preferably, a maximum of 6 —H atoms of a molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

The term “pharmaceutically acceptable” means approved by a regulatory agency such as the EMEA (Europe) and/or the FDA (US) and/or any other national regulatory agency for use in animals, preferably in humans.

In general the term “comprise” or “comprising” also encompasses “consist of” or “consisting of”.

The present invention relates to a hydrogel-linked prodrug and/or a pharmaceutical composition comprising a hydrogel-linked prodrug for use in the prevention, diagnosis and/or treatment of an ocular condition.

Preferred is the prevention and/or treatment of an ocular condition.

In another embodiment this invention relates to a hydrogel-linked prodrug and/or a pharmaceutical composition comprising a hydrogel-linked prodrug for use for intraocular injection. Preferably, the intraocular injection is an intravitreal injection into the vitreous body.

In a further embodiment the present invention relates to a hydrogel-linked prodrug and/or a pharmaceutical composition comprising a hydrogel-linked prodrug for use for intraocular injection in the prevention, diagnosis and/or treatment of an ocular condition. Preferably, the intraocular injection is an intravitreal injection into the vitreous body.

The ocular conditions to be prevented, diagnosed and/or treated with the pharmaceutical composition comprising hydrogel-linked prodrug can be divided into anterior ocular conditions and posterior ocular conditions.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site. Thus, an anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, retinal pigmented epithelium, Bruch's membrane, optic nerve (i.e. the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, nonretinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

In the hydrogel-linked prodrugs biologically active moieties are reversibly connected to the hydrogel of said hydrogel-linked prodrug through reversible prodrug linker moieties, and which biologically active moieties are released from said hydrogel-linked prodrug as drugs upon administration.

Preferably, the hydrogel of the hydrogel-linked prodrug is a biodegradable hydrogel.

The hydrogel comprises, preferably consists of at least one polymer which is preferably selected from the group of poly(acrylic acids), poly(acrylates), poly(acrylamides), poly(alkyloxy) polymers, poly(amides), poly(amidoamines), poly(amino acids), poly(anhydrides), poly(aspartamide), poly(butyric acid), poly(caprolacton), poly(carbonates), poly(cyanoacrylates), poly(dimethylacrylamide), poly(esters), poly(ethylene), poly(ethylene glycol), poly(ethylene oxide), poly(ethyloxazoline), poly(glycolic acid), poly(hydroxyethyl acrylate), poly(hydroxyethyloxazoline), poly(hydroxypropylmethacrylamide), poly(hydroxypropyl methacrylate), poly(hydroxypropyloxazoline), poly(iminocarbonates), poly(N-isopropylacrylamide), poly(lactic acid), poly(lactic-co-glycolic acid), poly(methacrylamide), poly(methacrylates), poly(methyloxazoline), poly(propylene fumarate), poly(organophosphazenes), poly(ortho esters), poly(oxazolines), poly(propylene glycol), poly(siloxanes), poly(urethanes), poly(vinylalcohols), poly(vinylamines), poly(vinylmethylether), poly(vinylpyrrolidone), silicones, ribonucleic acids, desoxynucleic acid, albumins, antibodies and fragments thereof, blood plasma protein, collagens, elastin, fascin, fibrin, keratins, polyaspartate, polyglutamate, prolamins, transferrins, cytochromes, flavoprotein, glycoproteins, hemoproteins, lipoproteins, metalloproteins, phytochromes, phosphoproteins, opsins, agar, agarose, alginate, arabinans, arabinogalactans, carrageenan, cellulose, carbomethyl cellulose, hydroxypropyl methylcellulose and other carbohydrate-based polymers, chitosan, dextran, dextrin, gelatin, hyaluronic acid and derivatives, mannan, pectins, rhamnogalacturonans, starch, hydroxyalkyl starch, xylan, and copolymers and functionalized derivatives thereof.

Preferably, the hydrogel is a biodegradable poly(ethylene glycol) (PEG)-based hydrogel. The hydrogel is a shaped article, preferably in the shape of microparticles. More preferably, the hydrogel is in the shape of microparticulate beads. Even more preferably, such microparticulate beads have a diameter of 1 to 1000 μm, more preferably of 5 to 500 μm, more preferably of 10 to 100 μm, even more preferably of 20 to 80 μm. Bead diameters are measured when the microparticulate beads are suspended in an isotonic aqueous buffer.

In a preferred embodiment, the hydrogel-linked prodrug is bead-shaped. More preferably, the hydrogel-linked prodrug is in the shape of microparticulate beads. Even more preferably, such microparticulate beads have a diameter of 1 to 1000 μm, more preferably of 5 to 500 μm, more preferably of 10 to 100 μm, even more preferably of 20 to 80 μm.

Bead diameters are measured when the microparticulate beads are suspended in an isotonic aqueous buffer.

Such hydrogel may be polymerized in different ways, such as through radical polymerization, ionic polymerization or ligation reactions. Preferred hydrogels, hydrogel-linked prodrugs and their methods of polymerization are disclosed in WO-A 2006/003014 and WO-A 2011/012715, which are hereby enclosed by reference in their entirety.

If the hydrogel is processed through radical or ionic polymerization, the at least two starting materials are crosslinking macromonomers or crosslinking monomers—which are referred to as crosslinker reagents—and a multi-functional macromonomer, which is referred to as backbone reagent. The crosslinker reagent carries at least two interconnectable functional groups and the backbone reagent carries at least one interconnectable functional group and at least one chemical functional group which is not intended to participate in the polymerization step. Additional diluent monomers may or may not be present.

Useful interconnectable functional groups include, but are not limited to, radically polymerizable groups, like vinyl, vinyl-benzene, acrylate, acrylamide, methacylate, methacrylamide and ionically polymerizable groups, like oxetane, aziridine, and oxirane.

In an alternative method of preparation, the hydrogel is generated through chemical ligation reactions. In such reactions, the starting material is at least one macromolecular starting material with complementary functionalities which undergo a reaction such as a condensation or addition reaction. In one embodiment, only one macromolecular starting material is used, which is a heteromultifunctional backbone reagent, comprising a number of polymerizable functional groups which may be the same or different.

In another embodiment and in the case if two or more macromolecular starting materials are used, one of these starting materials is a crosslinker reagent with at least two identical polymerizable functional groups and the other starting material is a homomultifunctional or heteromultifunctional backbone reagent, which also comprises a number of polymerizable functional groups.

Suitable polymerizable functional groups present on the crosslinker reagent include primary and secondary amines, carboxylic acid and derivatives, maleimide, thiol, hydroxyl and other alpha,beta unsaturated Michael acceptors, such as vinylsulfone groups, preferably terminal primary or secondary amine, carboxylic acid and derivatives, maleimide, thiol, hydroxyl and other alpha,beta unsaturated Michael acceptors, such as vinylsulfone groups. Suitable polymerizable functional groups present in the backbone reagent include, but are not limited to, primary and secondary amine, carboxylic acid and derivatives, maleimide, thiol, hydroxyl and other alpha,beta unsaturated Michael acceptors, like vinylsulfone groups.

The crosslinker reagent may be a linear or branched molecule and preferably is a linear molecule. If the crosslinker reagent has two polymerizable functional groups, it is referred to as a “linear crosslinker reagent”; if the crosslinker reagent has more than two polymerizable functional groups it is considered to be a “branched crosslinker reagent”.

Preferably, a crosslinker reagent is terminated by two polymerizable functional groups and may comprise no biodegradable group or may comprise at least one biodegradable bond. Preferably, the crosslinker reagent comprises at least one biodegradable bond.

In one embodiment, a crosslinker reagent consists of a polymer. Preferably, crosslinker reagents for hydrogel-linked prodrugs of drugs with a molecular weight of less than about 15 kDa have a molecular weight in the range of from 60 Da to 5 kDa, more preferably, from 0.5 kDa to 4 kDa, even more preferably from 1 kDa to 4 kDa, even more preferably from 1 kDa to 3 kDa. Preferably, crosslinker reagents for hydrogel-linked prodrugs of drugs with a molecular weight of more than about 15 kDa have a molecular weight in the range of from 2 to 40 kDa, more preferably of from 5 to 30 kDa, more preferably 2 to 20 kDa.

In addition to oligomeric or polymeric crosslinking reagents, low-molecular weight crosslinking reagents may be used, especially when hydrophilic high-molecular weight backbone moieties are used.

In one embodiment, a crosslinker reagent comprises monomers connected by biodegradable bonds, i.e. the crosslinker reagent is formed from monomers connected by biodegradable bonds. Such polymeric crosslinker reagents may contain up to 100 biodegradable bonds or more, depending on the molecular weight of the crosslinker reagent and the molecular weight of the monomer units. Examples for such crosslinker reagents may comprise poly(lactic acid)- or poly(glycolic acid)-based polymers.

Preferably, the crosslinker reagents are PEG based, preferably the crosslinker reagent is a PEG based molecular chain. Preferably, the poly(ethylene glycol) based crosslinker reagents are hydrocarbon chains comprising connected ethylene glycol units, wherein the poly(ethylene glycol) based crosslinker reagents comprise at least each m ethylene glycol units, and wherein m is an integer in the range of from 3 to 100, preferably from 10 to 70, if the drug has a molecular weight of less than about 15 kDa. If the drug has a molecular weight of more than about 15 kDa, m is an integer in the range of from 40 to 800, more preferably in the range of from 100 to 600 and most preferably in the range of from 100 to 400. Preferably, the poly(ethylene glycol) based crosslinker reagents have a molecular weight in the range of from 0.5 kDa to 5 kDa, if the drug is less than about 15 kDa, or in the range of from 5 to 30 kDa, if the drug has a molecular weight of more than about 15 kDa.

A preferred crosslinker reagent is shown below:

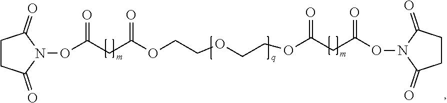

wherein
each m is independently an integer ranging from 2 to 4, and
q is an integer of from 3 to 100, if the hydrogel is used for a hydrogel-linked prodrug of drugs having a molecular weight of less than about 15 kDa and q is an integer of from 40 to 800, if the hydrogel is used for a hydrogel-linked prodrug of drugs having a molecular weight of more than about 15 kDa.

Even more preferred is the following crosslinker reagent:

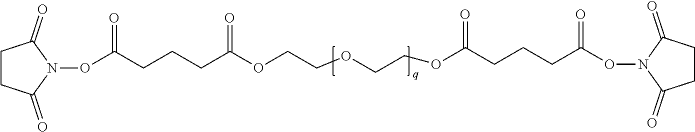

wherein q is 45.

Preferably, a backbone reagent is characterized by having a branching core, from which at least three PEG-based polymeric chains extend. Such branching cores may comprise, each in bound form, poly- or oligoalcohols, preferably pentaerythritol, tripentaerythritol, hexaglycerine, sucrose, sorbitol, fructose, mannitol, glucose, cellulose, amyloses, starches, hydroxyalkyl starches, polyvinylalcohols, dextranes, hyualuronans, or branching cores may comprise, each in bound form, mono-, poly- or oligoamines such as ornithine, diaminobutyric acid, trilysine, tetralysine, pentalysine, hexalysine, heptalysine, octalysine, nonalysine, decalysine, undecalysine, dodecalysine, tridecalysine, tetradecalysine, pentadecalysine or oligolysines, polyethyleneimines, polyvinylamines.

Preferably, three to sixteen PEG-based polymeric chains, more preferably four to eight PEG-based polymeric chains, extend from the branching core. Preferred branching cores may comprise, preferably consist of, pentaerythritol, trilysine, tetralysine, pentalysine, hexalysine, heptalysine or oligolysine, low-molecular weight PEI, hexaglycerine, or tripentaerythritol, each in bound form. Preferably, a PEG-based polymeric chain is a suitably substituted poly(ethylene glycol) derivative.

Preferably, such poly(ethylene glycol)-based polymeric chain is a linear PEG-based chain, of which one terminus is connected to the branching core and the other to a hyperbranched dendritic moiety. It is understood that a PEG-based chain may be terminated or interrupted by alkyl or aryl groups optionally substituted with heteroatoms and chemical functional groups.

Preferred backbone reagents comprising PEG-based polymeric chains extending from a branching core are multi-arm PEG derivatives as, for instance, detailed in the products list of JenKem Technology, USA (accessed by download from http://jenkemusa.net/pegproducts2.aspx on Mar. 8, 2011), such as a 4-arm-PEG derivative, in particular comprising a pentaerythritol core, an 8-arm-PEG derivative comprising a hexaglycerin core, and an 8-arm-PEG derivative comprising a tripentaerythritol core. Most preferred structures comprising PEG-based polymeric chains extending from a branching core suitable for backbone reagents are multi-arm PEG derivatives selected from:

a 4-arm PEG amine comprising a pentaerythritol core:

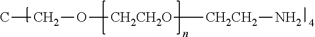

with n ranging from 5 to 500;

a 4-arm PEG carboxyl comprising a pentaerythritol core:

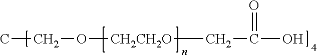

with n ranging from 5 to 500;

an 8-arm PEG amine comprising a hexaglycerin core:

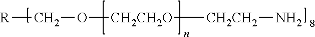

with n ranging from 5 to 500 and

R=hexaglycerin core structure;

an 8-arm PEG carboxyl comprising a hexaglycerin core:

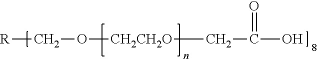

with n ranging from 5 to 500 and

R=hexaglycerin core structure;

an 8-arm PEG amine comprising a tripentaerythritol core:

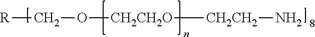

with n ranging from 5 to 500

and R=tripentaerythritol core structure;

and an 8-arm PEG carboxyl comprising a tripentaerythritol core:

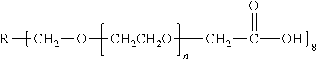

with n ranging from 5 to 500 and

R=tripentaerythritol core structure;

each in bound form.

Preferred molecular weights for such multi-arm PEG-derivatives in a backbone reagent comprising PEG-based polymeric chains extending from a branching core are 1 kDa to 20 kDa, more preferably 1 kDa to 15 kDa and even more preferably 1 kDa to 10 kDa. It is understood that the terminal amine groups are further conjugated to hyperbranched dendritic moieties.

The hyperbranched dendritic moiety of a backbone reagent provides polymerizable functional groups. Preferably, each dendritic moiety has a molecular weight in the range of from 0.4 kDa to 4 kDa, more preferably 0.4 kDa to 2 kDa. Preferably, each dendritic moiety has at least 3 branchings and at least 4 polymerizable functional groups, and at most 63 branchings and 64 polymerizable functional groups, preferred at least 7 branchings and at least 8 polymerizable functional groups and at most 31 branchings and 32 polymerizable functional groups.

Examples for such dendritic moieties are trilysine, tetralysine, pentalysine, hexalysine, heptalysine, octalysine, nonalysine, decalysine, undecalysine, dodecalysine, tridecalysine, tetradecalysine, pentadecalysine, hexadecalysine, heptadecalysine, octadecalysine, nonadecalysine, ornithine, and diaminobutyric acid in bound form. Preferred dendritic moieties are trilysine, tetralysine, pentalysine, hexalysine, heptalysine, each in bound form; most preferred are trilysine, pentalysine or heptalysine, each in bound form.

A preferred backbone reagent is the following:

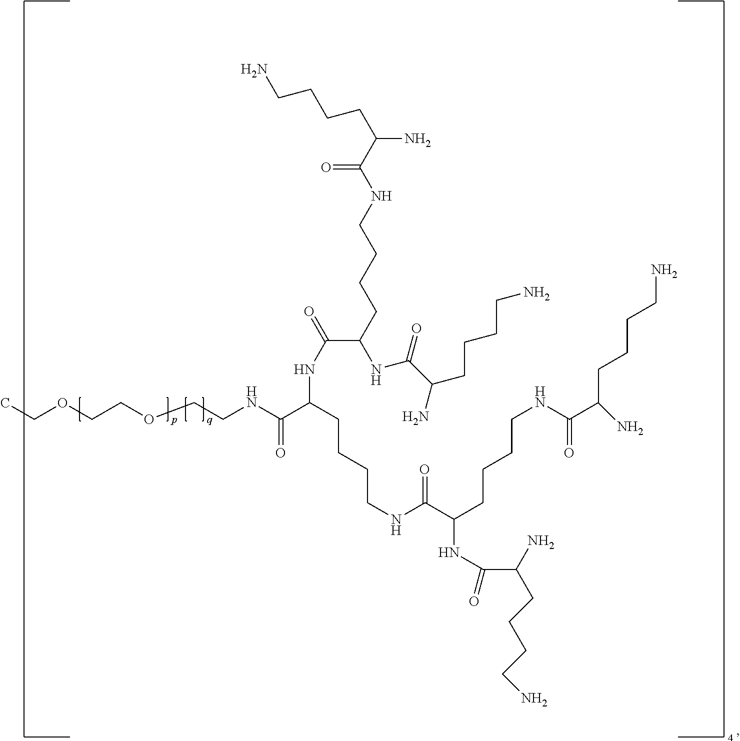

wherein
p is an integer of from 5 to 50, and
q is 1 or 2; and
wherein the —NH₂ moieties are the polymerizable functional groups of the backbone moiety.

During polymerization of the hydrogel, some polymerizable functional groups of the hyperbranched dendritic moieties are reacted with the polymerizable functional groups of crosslinker reagents to yield a reactive hydrogel to which further moieties are connected to provide hydrogel-linked prodrugs.

Polymerizable functional groups that participated in the polymerization process form the interconnected functional groups of the hydrogel. Polymerizable functional groups of the backbone reagents which did not participate in the polymerization reaction are referred to as reactive functional groups.

Ideally, the reactive functional groups are dispersed homogeneously throughout the reactive hydrogel, and may or may not be present on the surface of the reactive hydrogel. Non-limiting examples of such reactive functional groups include but are not limited to the following chemical functional groups connected to the hyperbranched dendritic moiety: carboxylic acid and activated derivatives, amino, maleimide, thiol and derivatives, sulfonic acid and derivatives, carbonate and derivatives, carbamate and derivatives, hydroxyl, aldehyde, ketone, hydrazine, isocyanate, isothiocyanate, phosphoric acid and derivatives, phosphonic acid and derivatives, haloacetyl, alkyl halides, acryloyl and other alpha-beta unsaturated michael acceptors, arylating agents like aryl fluorides, hydroxylamine, disulfides like pyridyl disulfide, vinyl sulfone, vinyl ketone, diazoalkanes, diazoacetyl compounds, oxirane, and aziridine. Preferred reactive functional groups include thiol, maleimide, amino, carboxylic acid and derivatives, carbonate and derivatives, carbamate and derivatives, aldehyde, and haloacetyl.

Preferably, the reactive functional groups are primary amino groups or carboxylic acids, most preferred primary amino groups.

Such reactive functional groups are characterized by being chemoselectively addressable in the presence of other functional groups and chemical functional groups.

The reactive functional groups may serve as attachment points for linkage of a spacer moiety, a reversible prodrug moiety or capping group. Spacer moieties are further connected to either reversible prodrug linker moieties or capping groups.

Preferably, the covalent attachment formed between a reactive functional group provided by a backbone moiety and a spacer moiety or a prodrug linker moiety is a permanent bond. Suitable reactive functional groups for attachment of a spacer moiety or a reversible prodrug linker moiety to the hydrogel include but are not limited to carboxylic acid and derivatives, carbonate and derivatives, hydroxyl, hydrazine, hydroxylamine, maleamic acid and derivatives, ketone, amino, aldehyde, thiol and disulfide.

A backbone moiety of the hydrogel is characterized by a number of hydrogel-connected biologically active moiety-reversible prodrug linker conjugates, hydrogel-connected spacer moieties, interconnected functional groups and optionally capping groups. Preferably, the sum of hydrogel-connected biologically active moiety-reversible prodrug linker conjugates, hydrogel-connected spacer moieties, interconnected functional groups and optionally capping groups per backbone moiety is 16 to 128, preferably 20 to 100, more preferably 24 to 80 and most preferably 30 to 60.

Preferably, the sum of hydrogel-connected biologically active moiety-reversible prodrug linker conjugates, hydrogel-connected spacer moieties, interconnected functional groups and optionally capping groups is equally divided by the number of PEG-based polymeric chains extending from the branching core. For instance, if there are 32 hydrogel-connected biologically active moiety-reversible prodrug linker conjugates, hydrogel-connected spacer moieties, interconnected functional groups and optionally capping groups, eight groups may be provided by each of the four PEG-based polymeric chains extending from the core by means of hyperbranched dendritic moieties attached to the terminus of each PEG-based polymeric chain. Alternatively, four functional groups may be provided by each of eight PEG-based polymeric chains extending from the core by means of hyperbranched dendritic moieties attached to the terminus of each PEG-based polymeric chain or two groups by each of sixteen PEG-based polymeric chains by means of hyperbranched dendritic moieties attached to the terminus of each PEG-based polymeric chain. If the number of PEG-based polymeric chains extending from the branching core does not allow for an equal distribution, it is preferred that the deviation from the mean number of the sum of hydrogel-connected biologically active moiety-reversible prodrug linker conjugates, interconnected functional groups and optionally capping groups per PEG-based polymeric chain is kept to a minimum.

Preferably, the reversible prodrug linker is attached to the biologically active moiety by an self-cleavable chemical functional group. Preferably, the linker has self-cleavable properties and as a consequence the hydrogel-linked prodrug is a carrier-linked prodrug, capable of releasing drug from the conjugate and in such a way that the release is predominantly dependent upon the self-cleavage of the linker.

Preferably, the linkage between reversible prodrug-linker and biologically active moiety is hydrolytically degradable under physiological conditions (aqueous buffer at pH 7.4, 37° C.) with half-lives ranging from one hour to nine months, include, but are not limited to, aconityls, acetals, amides, carboxlic anhydrides, esters, imines, hydrazones, maleamic acid amides, ortho esters, phosphamides, phosphoesters, phosphosilyl esters, silyl esters, sulfonic esters, aromatic carbamates, carbamates, sulfonamides, N-acetylsulfonamides, thiocarbamates, and combinations thereof, and the like. Preferred bonds and linkages which are non-enzymatically hydrolytically degradable or cleavable under physiological conditions (aqueous buffer at pH 7.4, 37° C.) with half-lives ranging from one hour to nine months are selected from aconityls, acetals, amides, carboxylic anhydrides, esters, imines, hydrazones, maleamic acid amides, ortho esters, phosphamides, phosphoesters, phosphosilyl esters, silyl esters, sulfonic esters, aromatic carbamates, and combinations thereof. Preferred biodegradable linkages between prodrug linker and biologically active moieties intended for transient linkage via a primary or aromatic hydroxyl group are esters, carbonates, phosphoesters and sulfonic acid esters and most preferred are esters or carbonates. Preferred biodegradable linkages between prodrug linker and biologically active moieties intended for transient linkage via a primary or aromatic amino group are amides or carbamates.

If the self-cleavable group is formed together with a primary or aromatic amino group of the biologically active moiety, a carbamate or amide group is preferred.

More preferably, the hydrogel is characterized in that the backbone moiety has a quaternary carbon of formula C(A-Hyp)₄, wherein each A is independently a poly(ethylene glycol)-based polymeric chain terminally attached to the quaternary carbon by a permanent covalent bond and the distal end of the PEG-based polymeric chain is covalently bound to a dendritic moiety Hyp, each dendritic moiety Hyp having at least four functional groups representing hydrogel-connected biologically active moiety-reversible prodrug linker conjugates, hydrogel-connected spacer moieties, interconnected functional groups and optionally capping groups.

Preferably, each A is independently selected from the formula —(CH₂)ₙ₁(OCH₂CH₂)ₙX—, wherein n1 is 1 or 2; n is an integer in the range of from 5 to 50; and X is a chemical functional group covalently linking A and Hyp.

Preferably, A and Hyp are covalently linked by an amide linkage.

Preferably, the dendritic moiety Hyp is a hyperbranched polypeptide. Preferably, the hyperbranched polypeptide is comprised of lysines in bound form. Preferably, each dendritic moiety Hyp has a molecular weight in the range of from 0.4 kDa to 4 kDa. It is understood that a backbone moiety C-(A-Hyp)₄ can consist of the same or different dendritic moieties Hyp and that each Hyp can be chosen independently. Each moiety Hyp consists of between 5 and 32 lysines, preferably of at least 7 lysines, i.e. each moiety Hyp is comprised of between 5 and 32 lysines in bound form, preferably of at least 7 lysines in bound form. Most preferably Hyp is comprised of heptalysinyl.

Preferably, there is a permanent amide bond between the hyperbranched dendritic moiety and the spacer moiety.

Preferably, C-(A-Hyp)₄ has a molecular weight in the range of from 1 kDa to 20 kDa, more preferably 1 kDa to 15 kDa and even more preferably 1 kDa to 10 kDa.

Such hydrogel, in particular biodegradable hydrogel, is characterized by a number of functional groups, consisting of hydrogel-connected biologically active moiety-reversible prodrug linker conjugates, hydrogel-connected spacer moieties, interconnected functional groups and optionally capping groups. Preferably, the sum of hydrogel-connected biologically active moiety-reversible prodrug linker conjugates, hydrogel-connected spacer moieties, interconnected functional groups and optionally capping groups is equal to or greater than 16, preferably 16 to 128, more preferably 20 to 100, even more preferred 20 to 80, even more preferably 24 to 32, most preferably 30-32.

The reactive functional groups of a reactive hydrogel serve as attachment points for hydrogel-connected biologically active moiety-reversible prodrug linker conjugates, hydrogel-connected spacer moieties, interconnected functional groups and optionally capping groups.

Such reactive hydrogel may be functionalized with a spacer carrying the same chemical functional group. For instance, amino groups may be introduced into such hydrogel by coupling a heterobifunctional spacer, such as suitably activated COOH-(EG)₆-NH-fmoc (EG=ethylene glycol), and removing the fmoc-protecting group. Such hydrogel can be further connected to a spacer carrying a different chemical functional group, such as a maleimide group. Such modified hydrogel may be further conjugated to biologically active moiety-reversible prodrug linker reagents, which carry a reactive thiol group on the reversible prodrug linker moiety.

In an alternative embodiment, multi-functional moieties are coupled to the reactive functional groups of the polymerized reactive biodegradable hydrogel to increase the number of reactive functional groups which allows for instance increasing the drug load of the hydrogel of the hydrogel-linked prodrug of the pharmaceutical composition of the present invention. Such multi-functional moieties may comprise lysine, dilysine, trilysine, tetralysine, pentalysine, hexalysine, heptalysine, or oligolysine, or low-molecular weight PEI, each in bound form. Preferably, the multi-functional moiety comprises lysine residues in bound form. Optionally, such multi-functional moiety may be protected with protecting groups and remaining reactive functional groups may be capped with suitable blocking reagents.

The covalent attachment formed between the reactive functional groups provided by such hydrogel and the reversible prodrug linker moieties are preferably permanent bonds. Suitable chemical functional groups for attachment of a reversible prodrug linker moiety to the reactive hydrogel include, but are not limited to, carboxylic acid and derivatives, carbonate and derivatives, hydroxyl, hydrazine, hydroxylamine, maleamic acid and derivatives, ketone, amino, aldehyde, thiol and disulfide.

A preferred backbone moiety is shown below, with dashed lines indicating interconnecting biodegradable linkages to crosslinker moieties:

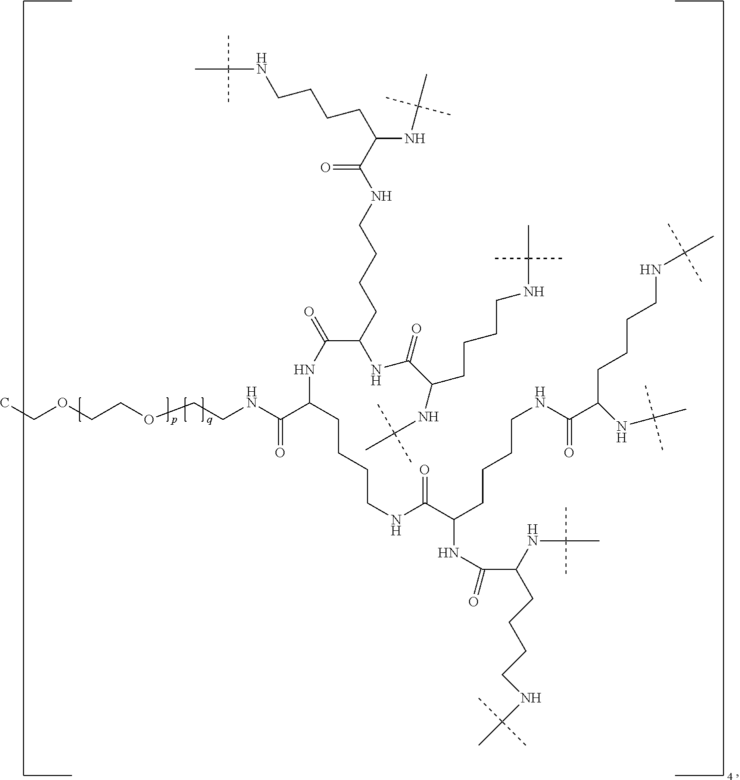

wherein
p is an integer of from 5 to 50, and
q is 1 or 2.

A preferred crosslinker moiety is shown below; dashed lines indicate interconnecting biodegradable linkages to backbone moieties:

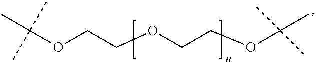

wherein n is an integer of from 5 to 50.

A particularly preferred carrier is a hydrogel obtainable by a process comprising the steps of:
(a) providing a mixture comprising
   (a-i) at least one backbone reagent, wherein the at least one backbone reagent has a molecular weight ranging from 1 to 100 kDa, and comprises at least three amines (—NH₂ and/or —NH—);
   (a-ii) at least one crosslinker reagent, wherein the at least one crosslinker reagent has a molecular weight ranging from 6 to 40 kDa, the at least one crosslinker reagent comprising
      (i) at least two carbonyloxy groups (—(C═O)—O— or —O—(C═O)—), and additionally
      (ii) at least two activated functional end groups selected from the group consisting of activated ester groups, activated carbamate groups, activated carbonate groups and activated thiocarbonate groups, and being PEG-based comprising at least 70% PEG; and
   (a-iii) a first solvent and at least a second solvent, which second solvent is immiscible in the first solvent, in a weight ratio of the at least one backbone reagent to the at least one crosslinker reagent ranging from 1:99 to 99:1;
(b) polymerizing the mixture of step (a) in a suspension polymerization to a hydrogel; and
(c) optionally working-up the hydrogel.

The mixture of step (a) comprises a first solvent and at least a second solvent. Said first solvent is preferably selected from the group comprising dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol and water and mixtures thereof.

The at least one backbone reagent and at least one crosslinker reagent are dissolved in the first solvent, i.e. the disperse phase of the suspension polymerization. In one embodiment the backbone reagent and the crosslinker reagent are dissolved separately, i.e. in different containers, using either the same or different solvent and preferably using the same solvent for both reagents. In another embodiment, the backbone reagent and the crosslinker reagent are dissolved together, i.e. in the same container and using the same solvent.

A suitable solvent for the backbone reagent is an organic solvent. Preferably, the solvent is selected from the group consisting of dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol and water and mixtures thereof. More preferably, the backbone reagent is dissolved in a solvent selected from the group comprising acetonitrile, dimethyl sulfoxide, methanol or mixtures thereof. Most preferably, the backbone reagent is dissolved in dimethylsulfoxide.

In one embodiment the backbone reagent is dissolved in the solvent in a concentration ranging from 1 to 300 mg/ml, more preferably from 5 to 60 mg/ml and most preferably from 10 to 40 mg/ml.

A suitable solvent for the crosslinker reagent is an organic solvent. Preferably, the solvent is selected from the group comprising dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol, water or mixtures thereof. More preferably, the crosslinker reagent is dissolved in a solvent selected from the group comprising dimethylformamide, acetonitrile, dimethyl sulfoxide, methanol or mixtures thereof. Most preferably, the crosslinker reagent is dissolved in dimethylsulfoxide.

In one embodiment the crosslinker reagent is dissolved in the solvent in a concentration ranging from 5 to 500 mg/ml, more preferably from 25 to 300 mg/ml and most preferably from 50 to 200 mg/ml.

The at least one backbone reagent and the at least one crosslinker reagent are mixed in a weight ratio ranging from 1:99 to 99:1, e.g. in a ratio ranging from 2:98 to 90:10, in a weight ratio ranging from 3:97 to 88:12, in a weight ratio ranging from 3:96 to 85:15, in a weight ratio ranging from 2:98 to 90:10 and in a weight ratio ranging from 5:95 to 80:20; particularly preferred in a weight ratio from 5:95 to 80:20, wherein the first number refers to the backbone reagent and the second number to the crosslinker reagent.

Preferably, the ratios are selected such that the mixture of step (a) comprises a molar excess of amine groups from the backbone reagent compared to the activated functional end groups of the crosslinker reagent. Consequently, the hydrogel resulting from the process of the present invention has free amine groups which can be used to couple a prodrug linker reagent to the hydrogel, either directly or through a spacer moiety.

The at least one second solvent, i.e. the continuous phase of the suspension polymerization, is preferably an organic solvent, more preferably an organic solvent selected from the group comprising linear, branched or cyclic C₅₋₃₀ alkanes; linear, branched or cyclic C₅₋₃₀ alkenes; linear, branched or cyclic C₅₋₃₀ alkynes; linear or cyclic poly(dimethylsiloxanes); aromatic C₆₋₂₀ hydrocarbons; and mixtures thereof. Even more preferably, the at least second solvent is selected from the group comprising linear, branched or cyclic C₅₋₁₆ alkanes; toluene; xylene; mesitylene; hexamethyldisiloxane; or mixtures thereof. Most preferably, the at least second solvent selected from the group comprising linear C₇₋₁₁ alkanes, such as heptane, octane, nonane, decane and undecane. Preferably, the mixture of step (a) further comprises a detergent. Preferred detergents are Cithrol DPHS, Hypermer 70A, Hypermer B246, Hypermer 1599A, Hypermer 2296, and Hypermer 1083.

Preferably, the detergent has a concentration of 0.1 g to 100 g per 1 L total mixture, i.e. disperse phase and continuous phase together. More preferably, the detergent has a concentration of 0.5 g to 10 g per 1 L total mixture, and most preferably, the detergent has a concentration of 0.5 g to 5 g per 1 L total mixture.

Preferably, the mixture of step (a) is an emulsion.

The polymerization in step (b) is initiated by adding a base. Preferably, the base is a non-nucleophilic base soluble in alkanes, more preferably the base is selected from N,N,N′,N′-tetramethylethylene diamine (TMEDA), 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, triethylamine, DIPEA, trimethylamine, N,N-dimethylethylamine, N,N,N′,N′-tetramethyl-1,6-hexanediamine, N,N,N′,N″,N″-pentamethyldiethylenetriamine, 1,8-diazabicyclo[5.4.0]undec-7-ene, 1,5-diazabicyclo[4.3.0]non-5-ene, and hexamethylenetetramine. Even more preferably, the base is selected from TMEDA, 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, 1,8-diazabicyclo[5.4.0]undec-7-ene, 1,5-diazabicyclo[4.3.0]non-5-ene, and hexamethylenetetramine. Most preferably, the base is TMEDA.

The base is added to the mixture of step (a) in an amount of 1 to 500 equivalents per activated functional end group in the mixture, preferably in an amount of 5 to 50 equivalents, more preferably in an amount of 5 to 25 equivalents and most preferably in an amount of 10 equivalents.

In process step (b), the polymerization of the hydrogel of the present invention is a condensation reaction, which preferably occurs under continuous stirring of the mixture of step (a). Preferably, the tip speed (tip speed=π×stirrer rotational speed×stirrer diameter) ranges from 0.2 to 10 meter per second (m/s), more preferably from 0.5 to 4 m/s and most preferably from 1 to 2 m/s.

In a preferred embodiment of step (b), the polymerization reaction is carried out in a cylindrical vessel equipped with baffles. The diameter to height ratio of the vessel may range from 4:1 to 1:2, more preferably the diameter to height ratio of the vessel ranges from 2:1 to 1:1.

Preferably, the reaction vessel is equipped with an axial flow stirrer selected from the group comprising pitched blade stirrer, marine type propeller, or Lightnin A-310. More preferably, the stirrer is a pitched blade stirrer.

Step (b) can be performed in a broad temperature range, preferably at a temperature from −10° C. to 100 C.°, more preferably at a temperature of 0° C. to 80° C., even more preferably at a temperature of 10° C. to 50° C. and most preferably at ambient temperature. “Ambient temperature” refers to the temperature present in a typical laboratory environment and preferably means a temperature ranging from 17 to 25° C.

Preferably, the hydrogel obtained from the polymerization is a shaped article, such as a coating, mesh, stent, nanoparticle or a microparticle. More preferably, the hydrogel is in the form of microparticular beads having a diameter from 1 to 500 micrometer, more preferably with a diameter from 10 to 300 micrometer, even more preferably with a diameter from 20 and 150 micrometer and most preferably with a diameter from 30 to 130 micrometer. The afore-mentioned diameters are measured when the hydrogel microparticles are fully hydrated in water.

Optional step (c) comprises one or more of the following step(s):

(c1) removing excess liquid from the polymerization reaction,

(c2) washing the hydrogel to remove solvents used during polymerization,

(c3) transferring the hydrogel into a buffer solution,

(c4) size fractionating/sieving of the hydrogel,

(c5) transferring the hydrogel into a container,

(c6) drying the hydrogel,

(c7) transferring the hydrogel into a specific solvent suitable for sterilization, and

(c8) sterilizing the hydrogel, preferably by gamma radiation

Preferably, optional step (c) comprises all of the following steps

(c1) removing excess liquid from the polymerization reaction,

(c2) washing the hydrogel to remove solvents used during polymerization,

(c3) transferring the hydrogel into a buffer solution,

(c4) size fractionating/sieving of the hydrogel,

(c5) transferring the hydrogel into a container,

(c7) transferring the hydrogel into a specific solvent suitable for sterilization, and

(c8) sterilizing the hydrogel, preferably by gamma radiation.

In one embodiment the backbone reagent is present in the form of its acidic salt, preferably in the form of an acid addition salt. Suitable acid addition salts are formed from acids which form non-toxic salts. Examples include but are not limited to the acetate, aspartate, benzoate, besylate, bicarbonate, carbonate, bisulphate, sulphate, borate, camsylate, citrate, edisylate, esylate, formate, fumarate, gluceptate, gluconate, glucuronate, hexafluorophosphate, hibenzate, hydrochloride, hydrobromide, hydroiodide, isethionate, lactate, malate, maleate, malonate, mesylate, methylsulphate, naphthylate, nicotinate, nitrate, orotate, oxalate, palmitate, pamoate, phosphate, hydrogen phosphate, dihydrogen phosphate, sacharate, stearate, succinate, tartrate and tosylate. Particularly preferred, the backbone reagent is present in the form of its hydrochloride salt.

In one embodiment, the at least one backbone reagent is selected from the group consisting of
a compound of formula (I)

B(-(A⁰)ₓ₁-(SP)ₓ₂-A¹-P-A²-Hyp¹)ₓ (I),
wherein
B is a branching core,
SP is a spacer moiety selected from the group consisting of C₁₋₆ alkyl, C₂₋₆ alkenyl and C₂₋₆ alkynyl,
P is a PEG-based polymeric chain comprising at least 80% PEG, preferably at least 85% PEG, more preferably at least 90% PEG and most preferably at least 95% PEG,
Hyp¹ is a moiety comprising an amine (—NH₂ and/or —NH—) or a polyamine comprising at least two amines (—NH₂ and/or —NH—),
x is an integer from 3 to 16,
x1, x2 are independently of each other 0 or 1, provided that x1 is 0, if x2 is 0,
A⁰, A¹, A² are independently of each other selected from the group consisting of

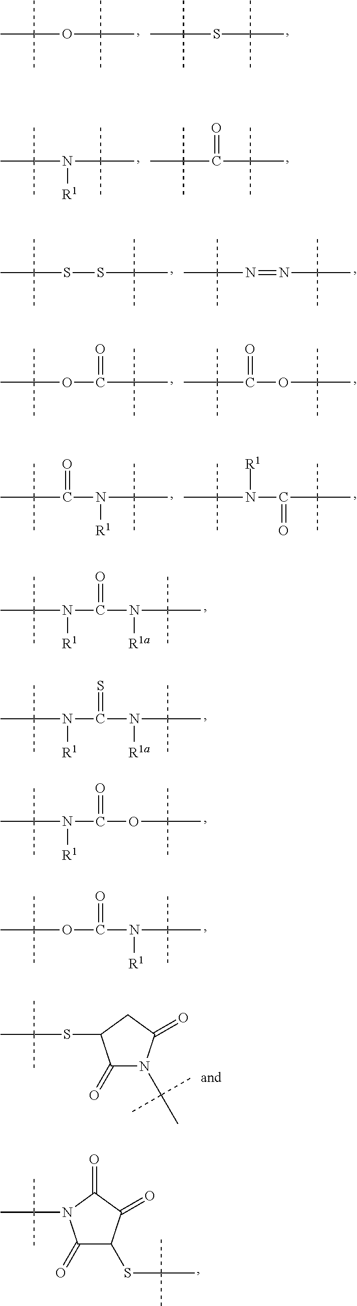

wherein R¹ and R^{1a} are independently of each other selected from H and C₁₋₆ alkyl;
a compound of formula (II)

Hyp²-A³-P-A⁴-Hyp³ (II),
wherein
P is defined as above in the compound of formula (I),
Hyp², Hyp³ are independently of each other a polyamine comprising at least two amines (—NH₂ and/or —NH—), and
A³ and A⁴ are independently selected from the group consisting of

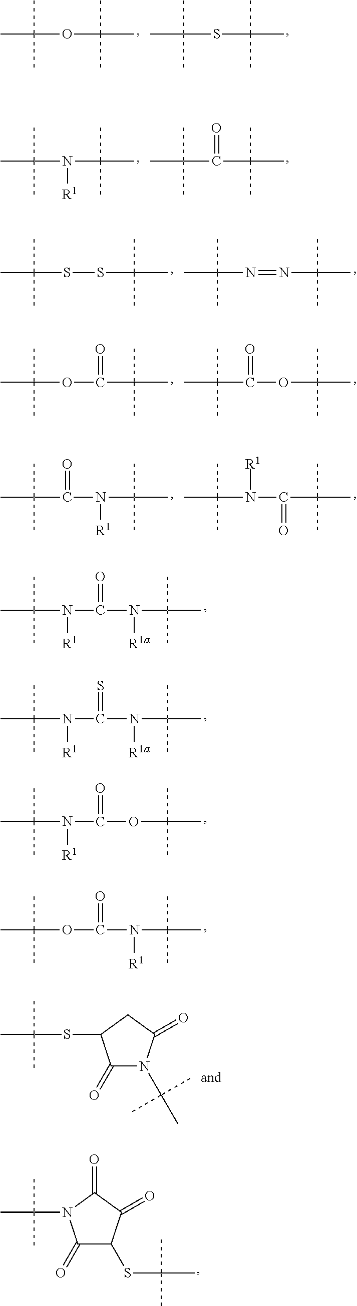

wherein R¹ and R^{1a} are independently of each other selected from H and C₁₋₆ alkyl;
a compound of formula (III)

P¹-A⁵-Hyp⁴ (III),
wherein
P¹ is a PEG-based polymeric chain comprising at least 80% PEG, preferably at least 85% PEG, more preferably at least 90% PEG and most preferably at least 95% PEG,
Hyp⁴ is a polyamine comprising at least three amines (—NH₂ and/or —NH), and
A⁵ is selected from the group consisting of

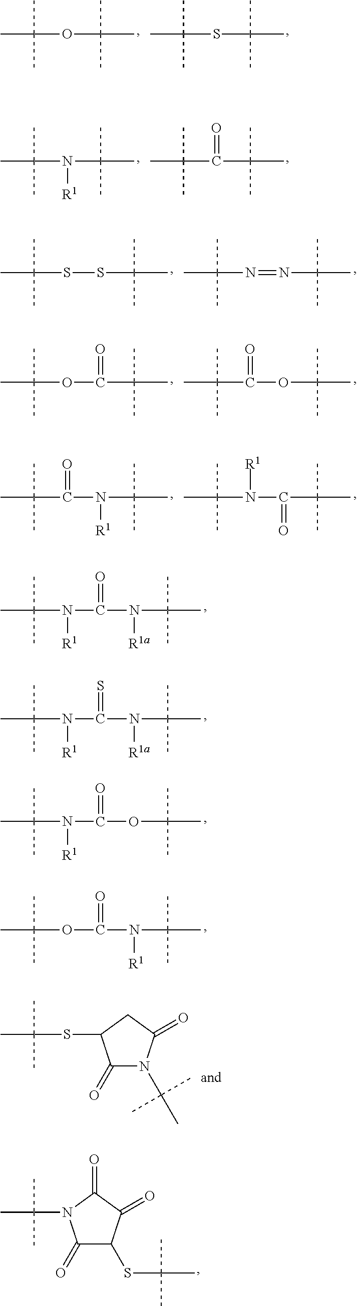

wherein R¹ and R^{1a} are independently of each other selected from H and C₁₋₆ alkyl;
and
a compound of formula (IV),

T¹-A⁶-Hyp⁵ (IV),
wherein
Hyp⁵ is a polyamine comprising at least three amines (—NH₂ and/or —NH), and
A⁶ is selected from the group consisting of

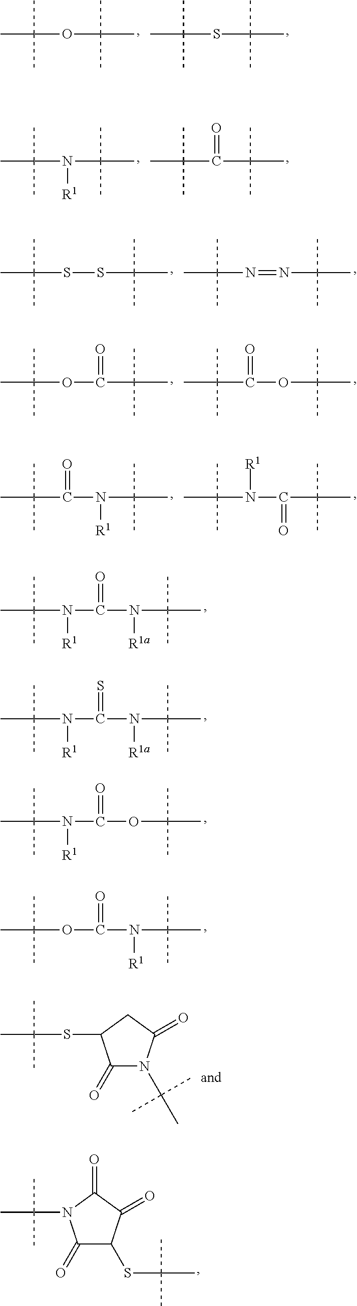

wherein R¹ and R^{1a} are independently of each other selected from H and C₁₋₆ alkyl; and
T¹ is selected from the group consisting of C₁₋₅₀ alkyl, C₂₋₅₀ alkenyl or C₂₋₅₀ alkynyl, which fragment is optionally interrupted by one or more group(s) selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl or naphthyl.

In the following sections the term “Hyp^{x}” refers to Hyp¹, Hyp², Hyp³, Hyp⁴ and Hyp⁵ collectively.

Preferably, the backbone reagent is a compound of formula (I), (II) or (III), more preferably the backbone reagent is a compound of formula (I) or (III), and most preferably the backbone reagent is a compound of formula (I).

In a preferred embodiment, in a compound of formula (I), x is 4, 6 or 8. Preferably, in a compound of formula (I) x is 4 or 8, most preferably, x is 4.

In a preferred embodiment in the compounds of the formulas (I) to (IV), A⁰, A¹, A², A³, A⁴, A⁵ and A⁶ are selected from the group comprising

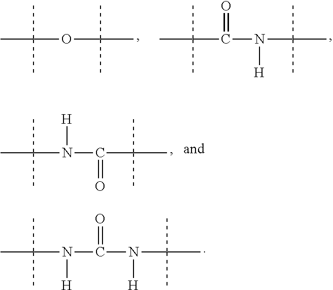

Preferably, in a compound of formula (I), A⁰ is

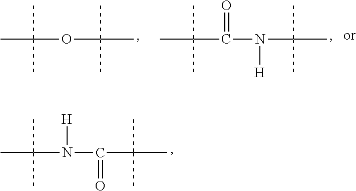

Preferably, in a compound of formula (I), A¹ is

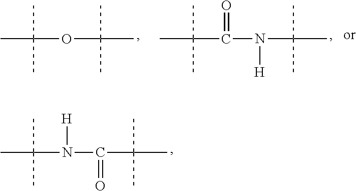

Preferably, in a compound of formula (I), A² is

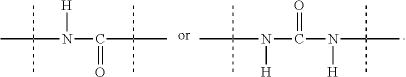

Preferably, in a compound of formula (II), A³ is

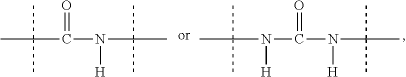

and A⁴ is

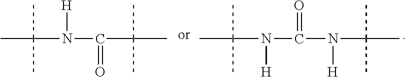

Preferably, in a compound of formula (III), A⁵ is

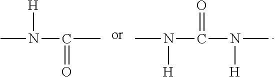

Preferably, in a compound of formula (IV), A⁶ is

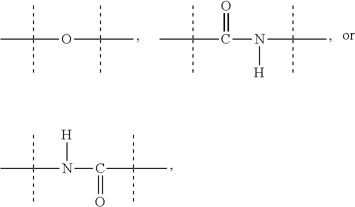

Preferably, in a compound of formula (IV), T¹ is selected from H and C₁₋₆ alkyl.

In one embodiment, in a compound of formula (I), the branching core B is selected from the following structures:

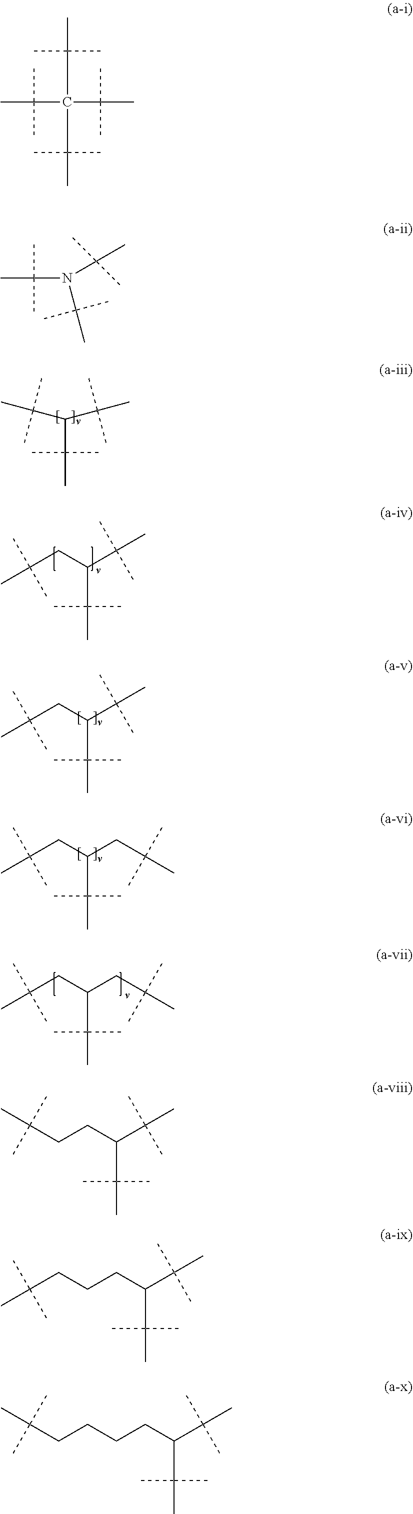

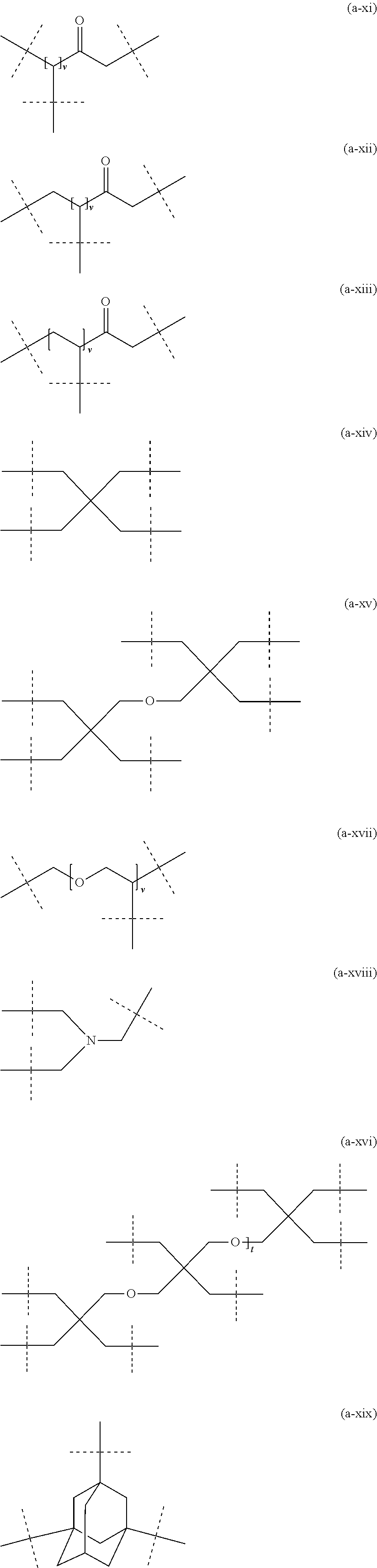

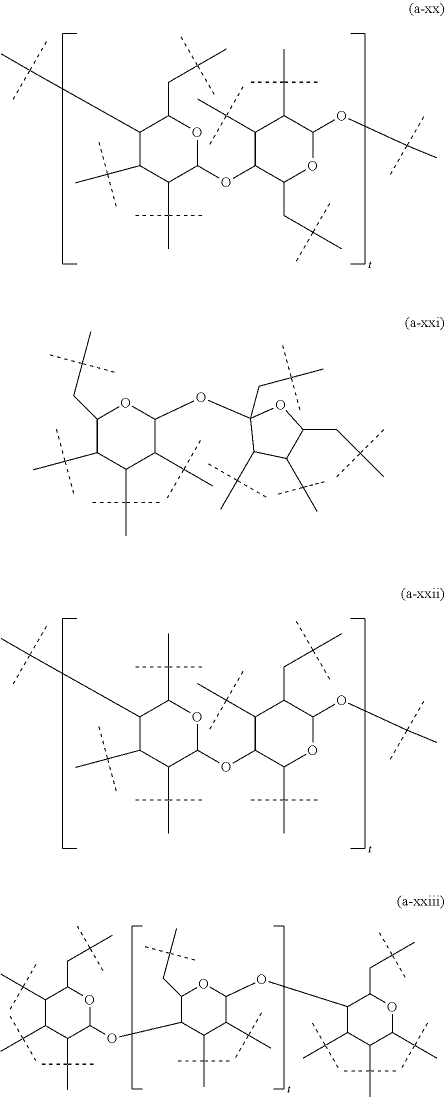

wherein
dashed lines indicate attachment to A⁰ or, if x1 and x2 are both 0, to A¹,
t is 1 or 2; preferably t is 1,
v is 1, 2, 3, 4, 5, 6, 7, 8, 9, 10, 11, 12, 13 or 14; preferably, v is 2, 3, 4, 5, 6; more preferably, v is 2, 4 or 6; most preferably, v is 2.

In a preferred embodiment, B has a structure of formula (a-i), (a-ii), (a-iii), (a-iv), (a-v), (a-vi), (a-vii), (a-viii), (a-ix), (a-x), (a-xiv), (a-xv) or (a-xvi). More preferably, B has a structure of formula (a-iii), (a-iv), (a-v), (a-vi), (a-vii), (a-viii), (a-ix), (a-x) or (a-iv). Most preferably, B has a structure of formula (a-xiv).

A preferred embodiment is a combination of B and A⁰, or, if x1 and x2 are both 0 a preferred combination of B and A¹, which is selected from the following structures:

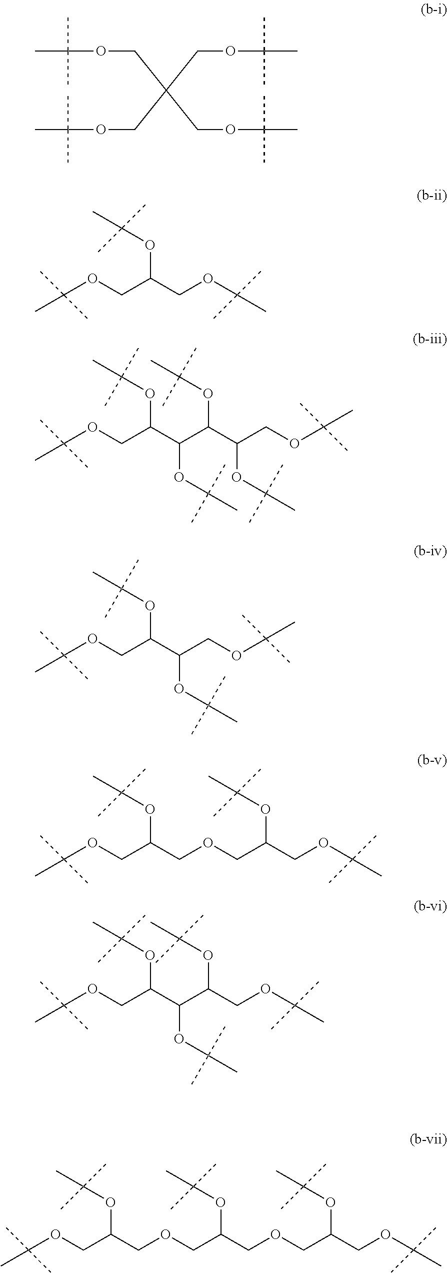

wherein
dashed lines indicate attachment to SP or, if x1 and x2 are both 0, to P.

More preferably, the combination of B and A⁰ or, if x1 and x2 are both 0, the combination of B and A¹, has a structure of formula of formula (b-i), (b-iv), (b-vi) or (b-viii) and most preferably has a structure of formula of formula (b-i).

In one embodiment, x1 and x2 of formula (I) are 0.

In one embodiment, the PEG-based polymeric chain P has a molecular weight from 0.3 kDa to 40 kDa; e.g. from 0.4 to 35 kDa, from 0.6 to 38 kDA, from 0.8 to 30 kDa, from 1 to 25 kDa, from 1 to 15 kDa or from 1 to 10 kDa. Most preferably P has a molecular weight from 1 to 10 kDa.

In one embodiment, the PEG-based polymeric chain P¹ has a molecular weight from 0.3 kDa to 40 kDa; e.g. from 0.4 to 35 kDa, from 0.6 to 38 kDA, from 0.8 to 30 kDa, from 1 to 25 kDa, from 1 to 15 kDa or from 1 to 10 kDa. Most preferably P¹ has a molecular weight from 1 to 10 kDa.

In one embodiment, in the compounds of formulas (I) or (II), P has the structure of formula (c-i):

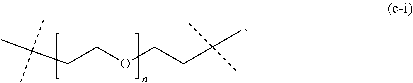

wherein n ranges from 6 to 900, more preferably n ranges from 20 to 700 and most preferably n ranges from 20 to 250.

In one embodiment, in the compounds of formulas (III), P¹ has the structure of formula (c-ii):

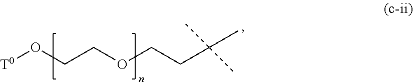

wherein
n ranges from 6 to 900, more preferably n ranges from 20 to 700 and most preferably n ranges from 20 to 250;
T⁰ is selected from the group comprising C₁₋₆ alkyl, C₂₋₆ alkenyl and C₂₋₆ alkynyl, which is optionally interrupted by one or more group(s) selected from —NH—, —N(C₁₋₄ alkyl)-, —O—, —S—, —C(O)—, —C(O)NH—, —C(O)N(C₁₋₄ alkyl)-, —O—C(O)—, —S(O)— or —S(O)₂—.

In one embodiment, in the compounds of formulas (I) to (IV), the moiety Hyp^{x} is a polyamine and preferably comprises in bound form and, where applicable, in R- and/or S-configuration a moiety of the formulas (d-i), (d-ii), (d-iii) and/or (d-iii):

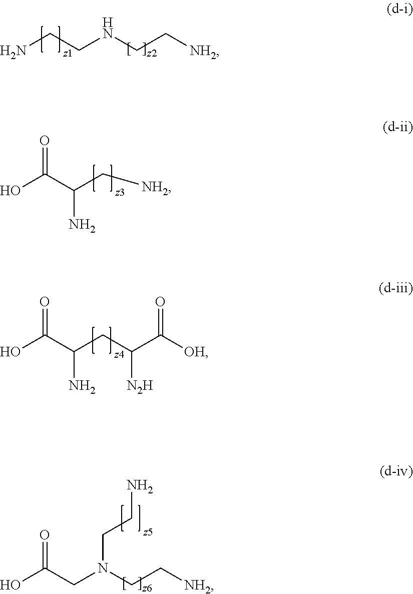

wherein
z1, z2, z3, z4, z5, z6 are independently of each other 1, 2, 3, 4, 5, 6, 7 or 8.

More preferably, Hyp^{x} comprises in bound form and in R- and/or S-configuration lysine, ornithine, diaminoproprionic acid and/or diaminobutyric acid.

Hyp^{x} has a molecular weight from 40 Da to 30 kDa, preferably from 0.3 kDa to 25 kDa, more preferably from 0.5 kDa to 20 kDa.

Hyp^{x} is preferably selected from the group consisting of

a moiety of formula (e-i)

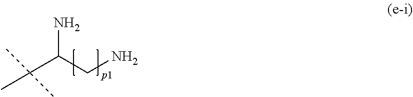

wherein
p1 is an integer from 1 to 5, preferably p1 is 4, and
the dashed line indicates attachment to A² if the backbone reagent has a structure of formula (I) and to A³ or A⁴ if the backbone reagent has the structure of formula (II);

a moiety of formula (e-ii)

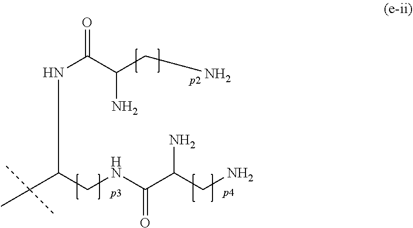

wherein
p2, p3 and p4 are identical or different and each is independently of the others an integer from 1 to 5, preferably p2, p3 and p4 are 4, and
the dashed line indicates attachment to A² if the backbone reagent has a structure of formula (I), to A³ or A⁴ if the backbone reagent has a structure of formula (II), to A⁵ if the backbone reagent has a structure of formula (III) and to A⁶ if the backbone reagent has a structure of formula (IV);

a moiety of formula (e-iii)

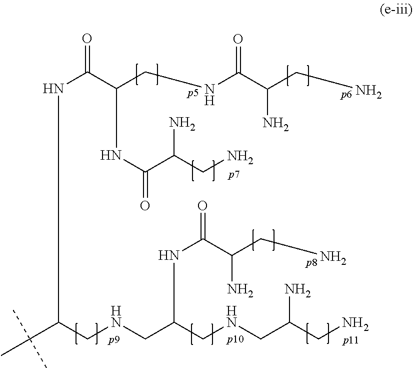

wherein
p5 to p11 are identical or different and each is independently of the others an integer from 1 to 5, preferably p5 to p11 are 4, and
the dashed line indicates attachment to A² if the backbone reagent is of formula (I), to A³ or A⁴ if the backbone reagent is of formula (II), to A⁵ if the backbone reagent is of formula (III) and to A⁶ if the backbone reagent is of formula (IV);

a moiety of formula (e-iv)

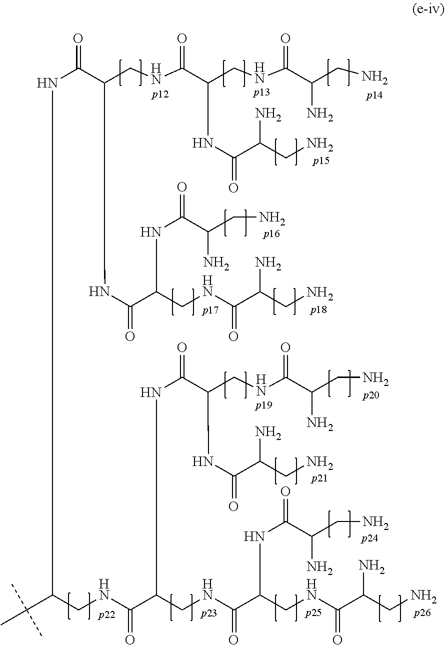

wherein
p12 to p26 are identical or different and each is independently of the others an integer from 1 to 5, preferably p12 to p26 are 4, and the dashed line indicates attachment to A² if the backbone reagent has a structure of formula (I), to A³ or A⁴ if the backbone reagent has a structure of formula (II), to A⁵ if the backbone reagent has a structure of formula (III) and to A⁶ if the backbone reagent has a structure of formula (IV);

a moiety of formula (e-v)

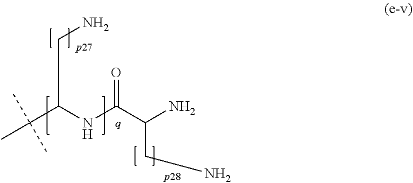

wherein
p27 and p28 are identical or different and each is independently of the other an integer from 1 to 5, preferably p27 and p28 are 4,
q is an integer from 1 to 8, preferably q is 2 or 6 and most preferably 1 is 6, and
the dashed line indicates attachment to A² if the backbone reagent has a structure of formula (I), to A³ or A⁴ if the backbone reagent has a structure of formula (II), to A⁵ if the backbone reagent has a structure of formula (III) and to A⁶ if the backbone reagent has a structure of formula (IV);

a moiety of formula (e-vi)

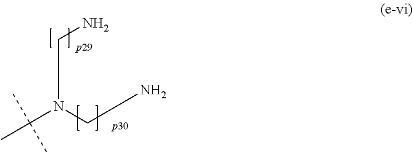

wherein
p29 and p30 are identical or different and each is independently of the other an integer from 2 to 5, preferably p29 and p30 are 3, and
the dashed line indicates attachment to A² if the backbone reagent has the structure of formula (I), to A³ or A⁴ if the backbone reagent has the structure of formula (II), to A⁵ if the backbone reagent has the structure of formula (III) and to A⁶ if the backbone reagent has the structure of formula (IV);

a moiety of formula (e-vii)

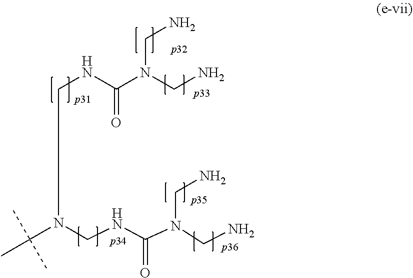

wherein
p31 to p36 are identical or different and each is independently of the others an integer from 2 to 5, preferably p31 to p36 are 3, and
the dashed line indicates attachment to A² if the backbone reagent has a structure of formula (I), to A³ or A⁴ if the backbone reagent has a structure of formula (II), to A⁵ if the backbone reagent has a structure of formula (III) and to A⁶ if the backbone reagent has a structure of formula (IV);

a moiety of formula (e-viii)

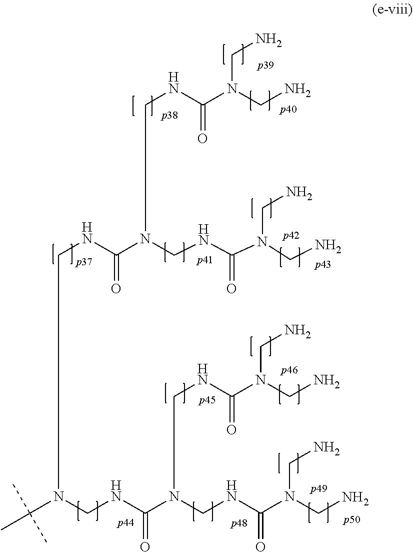

wherein
p37 to p50 are identical or different and each is independently of the others an integer from 2 to 5, preferably p37 to p50 are 3, and
the dashed line indicates attachment to A² if the backbone reagent has a structure of formula (I), to A³ or A⁴ if the backbone reagent has a structure of formula (II), to A⁵ if the backbone reagent has a structure of formula (III) and to A⁶ if the backbone reagent has a structure of formula (IV); and a moiety of formula (e-ix):

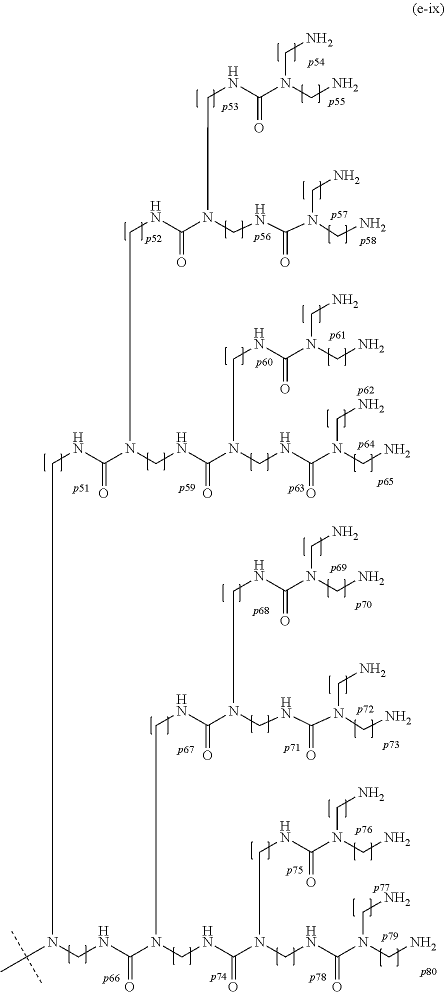

wherein
p51 to p80 are identical or different and each is independently of the others an integer from 2 to 5, preferably p51 to p80 are 3, and
the dashed line indicates attachment to A² if the backbone reagent has a structure of formula (I), to A³ or A⁴ if the backbone reagent has a structure of formula (II), to A⁵ if the backbone reagent has a structure of formula (III) and to A⁶ if the backbone reagent has a structure of formula (IV); and

wherein the moieties (e-i) to (e-v) may at each chiral center be in either R- or S-configuration, preferably, all chiral centers of a moiety (e-i) to (e-v) are in the same configuration.

Preferably, Hyp^{x} is has a structure of formulas (e-i), (e-ii), (e-iii), (e-iv), (e-vi), (e-vii), (e-viii) or (e-ix). More preferably, Hyp^{x} has a structure of formulas (e-ii), (e-iii), (e-iv), (e-vii), (e-viii) or (e-ix), even more preferably Hyp^{x} has a structure of formulas (e-ii), (e-iii), (e-vii) or (e-viii) and most preferably Hyp^{x} has the structure of formula (e-iii).

If the backbone reagent has a structure of formula (I), a preferred moiety -A²-Hyp¹ is a moiety of the formula

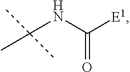

wherein
the dashed line indicates attachment to P; and
E¹ is selected from formulas (e-i) to (e-ix).

If the backbone reagent has a structure of formula (II) a preferred moiety Hyp²-A³- is a moiety of the formula

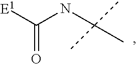

wherein
the dashed line indicates attachment to P; and
E¹ is selected from formulas (e-i) to (e-ix);

and a preferred moiety -A⁴-Hyp³ is a moiety of the formula

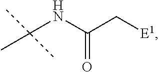

wherein
the dashed line indicates attachment to P; and
E¹ is selected from formulas (e-i) to (e-ix).

If the backbone reagent has a structure of formula (III), a preferred moiety -A⁵-Hyp⁴ is a moiety of the formula

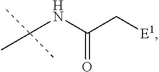

wherein
the dashed line indicates attachment to P¹; and
E¹ is selected from formulas (e-i) to (e-ix).

More preferably, the backbone reagent has a structure of formula (I) and B is has a structure of formula (a-xiv).

Even more preferably, the backbone reagent has the structure of formula (I), B has the structure of formula (a-xiv), x1 and x2 are 0, and A¹ is —O—.

Even more preferably, the backbone reagent has the structure of formula (I), B has the structure of formula (a-xiv), A¹ is —O—, and P has a structure of formula (c-i).

Most preferably, the backbone reagent has the following formula:

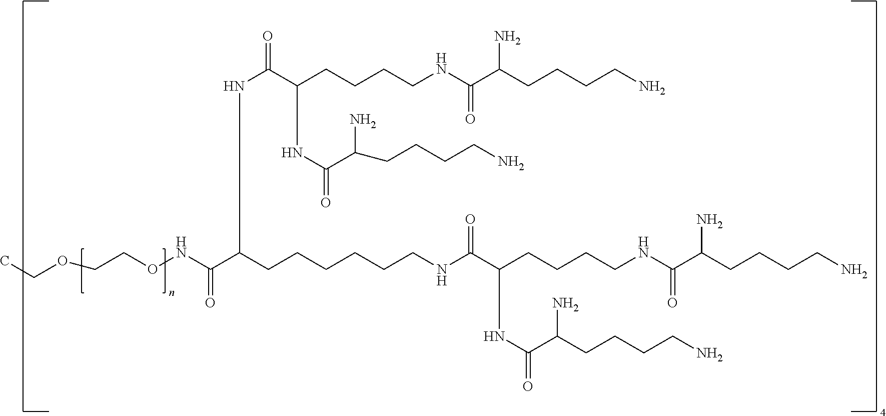

wherein
n ranges from 10 to 40, preferably from 10 to 30, more preferably from 10 to 20.

SP is a spacer moiety selected from the group comprising C₁₋₆ alkyl, C₂₋₆ alkenyl and C₂₋₆ alkynyl, preferably SP is —CH₂—, —CH₂—CH₂—, —CH(CH₃)—, —CH₂—CH₂—CH₂—, —CH(C₂H₅)—, —C(CH₃)₂—, —CH═CH— or —CH═CH—, most preferably SP is —CH₂—, —CH₂—CH₂— or —CH═CH—.

The at least one crosslinker reagent comprises at least two carbonyloxy groups (—(C═O)—O— or —O—(C═O)—), which are biodegradable linkages. These biodegradable linkages are necessary to render the hydrogel biodegradable. Additionally, the at least one crosslinker reagent comprises at least two activated functional end groups which during the polymerization of step (b) react with the amines of the at least one backbone reagent.

The crosslinker reagent has a molecular weight ranging from 6 to 40 kDa, more preferably ranging from 6 to 30 kDa, even more preferably ranging from 6 to 20 kDa, even more preferably ranging from 6 to 15 kDa and most preferably ranging from 6 to 10 kDa.

The crosslinker reagent comprises at least two activated functional end groups selected from the group comprising activated ester groups, activated carbamate groups, activated carbonate groups and activated thiocarbonate groups, which during polymerization react with the amine groups of the backbone reagents, forming amide bonds.

Preferably, the crosslinker reagent is a compound of formula (V):

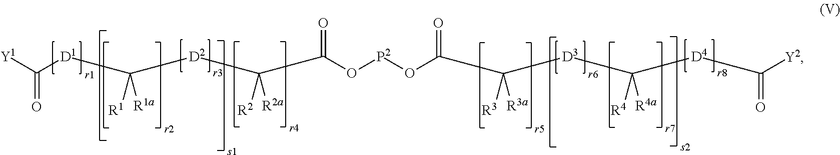

wherein
D¹, D², D³ and D⁴ are identical or different and each is independently of the others selected from the group comprising O, NR⁵, S and CR⁵R^{5a};
R¹, R^{1a}, R², R², R³, R^{3a}, R⁴, R^{4a}, R⁵ and R^{5a} are identical or different and each is independently of the others selected from the group comprising H and C₁₋₆ alkyl; optionally, one or more of the pair(s) R¹/R^{1a}, R²/R^{2a}, R³R^{3a}, R⁴/R^{4a}, R¹/R², R³/R⁴, R^{1a}/R^{2a}, and R^{3a}/R^{4a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl;
A is selected from the group consisting of phenyl, naphthyl, indenyl, indanyl and tetralinyl;
P² is

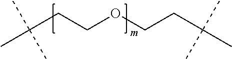

m ranges from 120 to 920, preferably from 120 to 460 and more preferably from 120 to 230;
r1, r2, r7, r8 are independently 0 or 1;
r3, r6 are independently 0, 1, 2, 3, or 4;
r4, r5 are independently 1, 2, 3, 4, 5, 6, 7, 8, 9 or 10;
s1, s2 are independently 1, 2, 3, 4, 5 or 6;
Y¹, Y² are identical or different and each is independently of the other selected from formulas (f-i) to (f-vi):

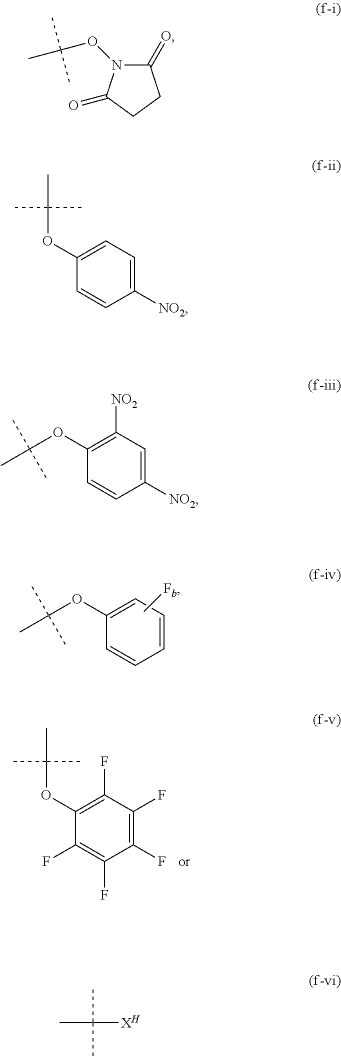

wherein
the dashed lines indicate attachment to the rest of the molecule,
b is 1, 2, 3 or 4
X^{H} is Cl, Br, I, or F.

It is understood that the Y¹ and Y² represent the at least two activated functional end groups.

Preferably, Y¹ and Y² have a structure of formula (f-i), (f-ii) or (f-v). More preferably, Y¹ and Y² have a structure of formula (f-i) or (f-ii) and most preferably, Y¹ and Y² have a structure of formula (f-i).

Preferably, both moieties Y¹ and Y² have the same structure. More preferably, both moieties Y¹ and Y² have the structure of formula (f-i).

Preferably, r1 is 0.

Preferably, r1 and s1 are both 0.

Preferably, one or more of the pair(s) R¹/R^{1a}, R²/R^{2a}, R³/R^{3a}, R⁴/R^{4a}, R¹/R², R³/R⁴, R^{1a}/R^{2a}, and R^{3a}/R^{4a} form a chemical bond or are joined together with the atom to which they are attached to form a C₃₋₈ cycloalkyl or form a ring A.

Preferably, one or more of the pair(s) R¹/R², R^{1a}/R^{2a}, R³/R⁴, R^{3a}/R^{4a} are joined together with the atom to which they are attached to form a 4- to 7-membered heterocyclyl or 8- to 11-membered heterobicyclyl.

Preferably, the crosslinker reagent of formula (V) is symmetric, i.e. the moiety

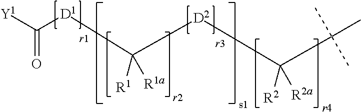

has the same structure as the moiety

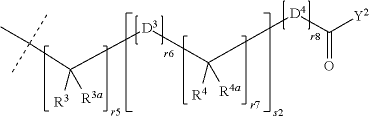

Preferred crosslinker reagents are of formula (V-1) to (V-53):

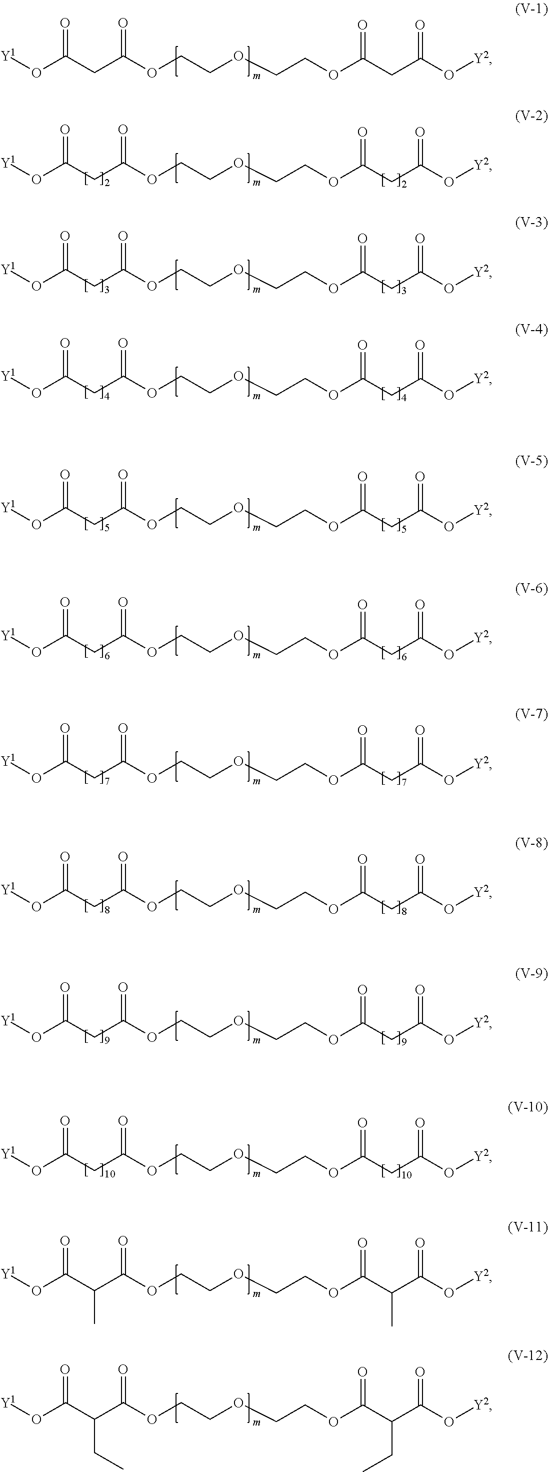

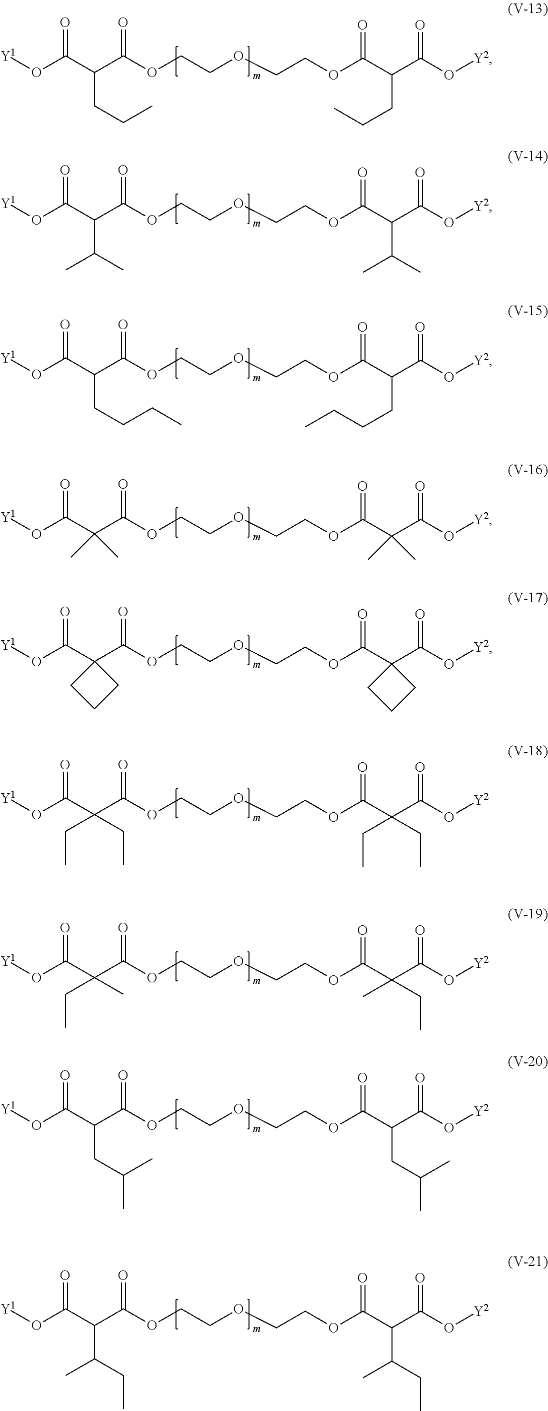

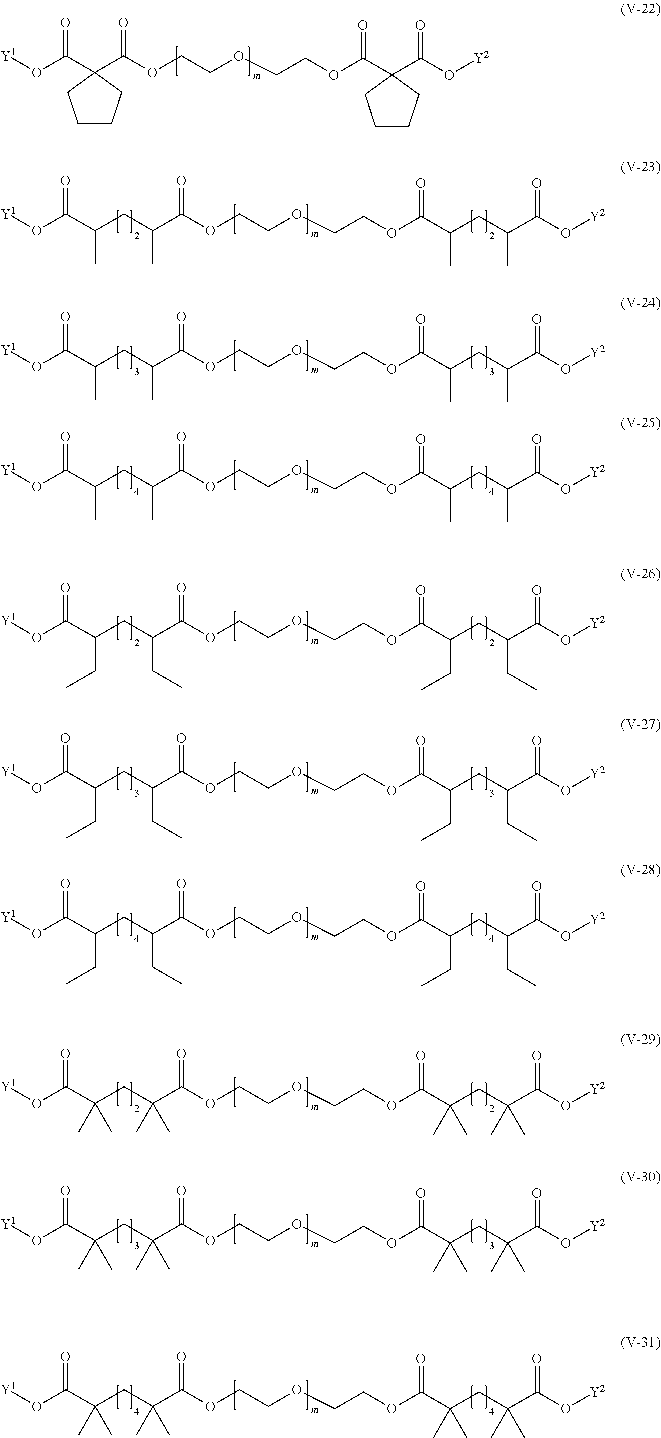

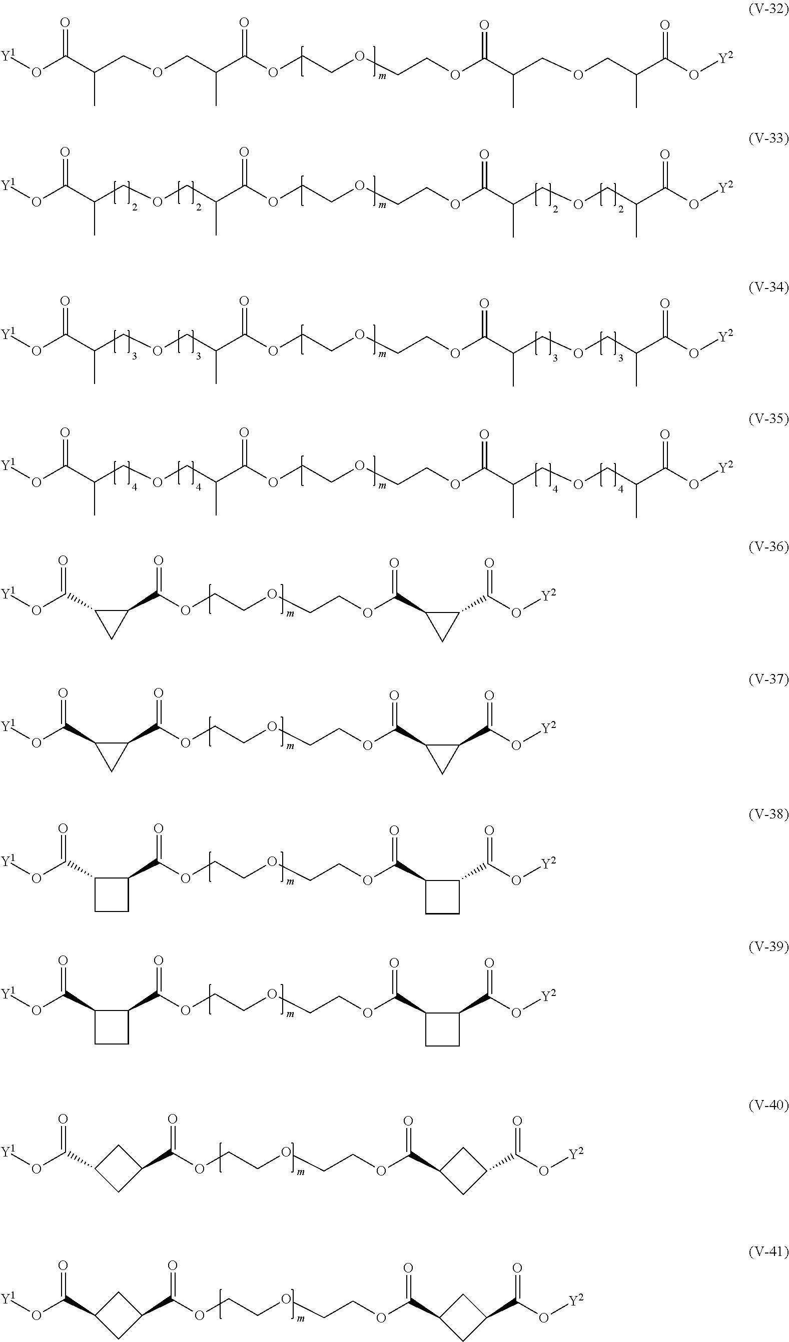

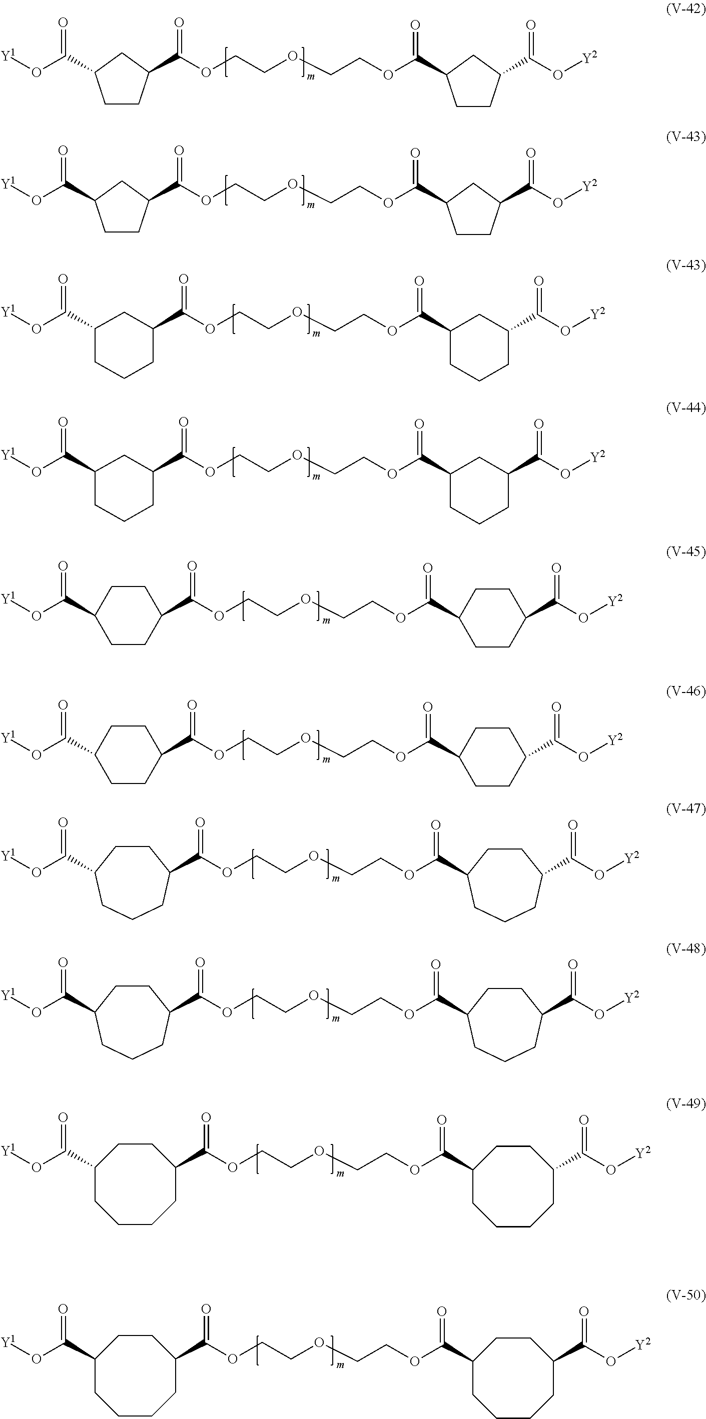

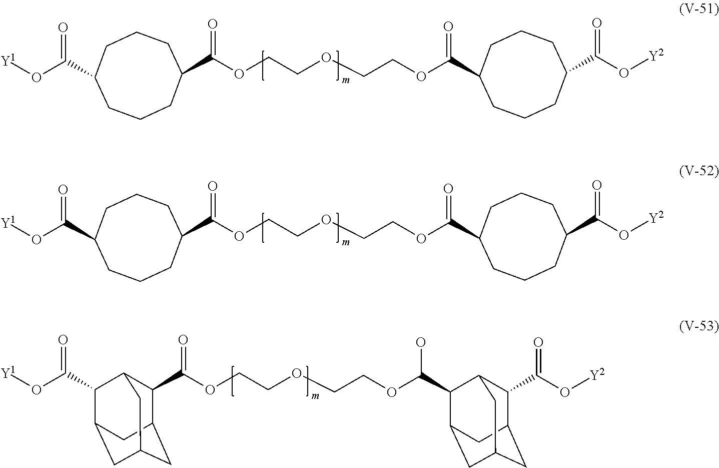

wherein
each crosslinker reagent may be in the form of its racemic mixture, where applicable; and
m, Y¹ and Y² are defined as above.

It was surprisingly found that the use of crosslinker reagents with branches, i.e. residues other than H, at the alpha carbon of the carbonyloxy group lead to the formation of hydrogels which are more resistant against enzymatic degradation, such as degradation through esterases.

Similarly, it was surprisingly found that the fewer atoms there are between the (C═O) of a carbonyloxy group and the (C═O) of the adjacent activated ester, activated carbamate, activated carbonate or activated thiocarbamate, the more resistant against degradation the resulting hydrogels are, such as more resistant against degradation through esterases. Accordingly, crosslinker reagents V-11 to V-53, V-1 and V-2 are preferred crosslinker reagents.

The preferred embodiments of the compound of formula (V) as mentioned above apply accordingly to the preferred compounds of formulas (V-1) to (V-53).

In another aspect, the present invention relates to a hydrogel obtainable by a process of the present invention as defined above.

The hydrogel contains from 0.01 to 1 mmol/g primary amine groups (—NH₂), more preferably, from 0.02 to 0.5 mmol/g primary amine groups and most preferably from 0.05 to 0.3 mmol/g primary amine groups. The term “X mmol/g primary amine groups” means that 1 g of dry hydrogel comprises X mmol primary amine groups. Measurement of the amine content of the hydrogel may be carried out according to Gude et al. (Letters in Peptide Science, 2002, 9(4): 203-206, which is incorporated by reference in its entirety).

A biologically active moiety is connected to the hydrogel of the hydrogel-linked prodrug through a reversible prodrug linker. The reversible prodrug linkers of a hydrogel-linked prodrug may be the same or different. Preferably, the reversible prodrug linkers of the hydrogel-linked prodrug are the same.

A suitable reversible prodrug linker moiety may be chosen depending on the one or more chemical functional groups present in the corresponding drug of a biologically active moiety. Suitable reversible prodrug linker moieties are known to the person skilled in the art and preferred examples are given in the following sections.

In a preferred embodiment, the reversible prodrug linker moiety connecting the hydrogel to a biologically active moiety is a traceless prodrug linker. Preferably, all reversible prodrug linker moieties of the hydrogel-linked prodrug are traceless prodrug linkers.

A preferred reversible prodrug linker moiety for amine-comprising drugs is described in WO-A 2005/099768. Therefore, the following sub-structures selected from the general formulas (II) and (III) are preferred embodiments for reversible prodrug linker-biologically active moiety conjugates:

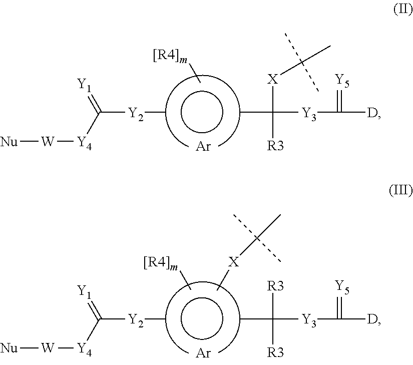

wherein the dashed line indicates attachment to the hydrogel or to a spacer moiety which is connected to the hydrogel, and wherein X, Y₁, Y₂, Y₃, Y₄, Y₅, R2, R3, R4, Nu, W, m, and D of formulas (II) and (III) have the following meaning:
D is an amine-comprising biologically active moiety which is attached to the rest of the sub-structure shown in formula (II) or (III) by forming a —O—(C═O)—N—; —O—(C═S)—N—; —S—(C═O)—N—; or —S—(C═S)—N— linkage;
X is a spacer moiety R5-Y6;
Y₁ and Y₂ are each independently O, S or NR6;
Y₃ is O or S;
Y₄ is O, NR6, or —C(R7)(R8)-;
Y₅ is 0 or S;
Y6 is O, S, NR6, succinimide, maleimide, unsaturated carbon-carbon bonds or any heteroatom containing a free electron pair or is absent;
R2 and R3 are independently selected from the group consisting of hydrogen, substituted or unsubstituted linear, branched or cyclical alkyl or heteroalkyl groups, aryls, substituted aryls, substituted or unsubstituted heteroaryls, cyano groups, nitro groups, halogens, carboxy groups, carboxyalkyl groups, alkylcarbonyl groups and carboxamidoalkyl groups;
R4 is selected from the group consisting of hydrogen, substituted or unsubstituted linear, branched or cyclical alkyls or heteroalkyls, aryls, substituted aryls, substituted or unsubstituted heteroaryl, substituted or unsubstituted linear, branched or cyclical alkoxys, substituted or unsubstituted linear, branched or cyclical heteroalkyloxys, aryloxys or heteroaryloxys, cyano groups and halogens;
R5 is selected from substituted or non-substituted linear, branched or cyclical alkyl or heteroalkyl, aryls, substituted aryls, substituted or non-substituted heteroaryls;
R6 is selected from hydrogen, substituted or unsubstituted linear, branched or cyclical alkyls or heteroalkyls, aryls, substituted aryls and substituted or unsubstituted heteroaryls;
R7 and R8 are each independently selected from the group consisting of hydrogen, substituted or unsubstituted linear, branched or cyclical alkyls or heteroalkyls, aryls, substituted aryls, substituted or unsubstituted heteroaryls, carboxyalkyl groups, alkylcarbonyl groups, carboxamidoalkyl groups, cyano groups, and halogens;
W is selected from substituted or unsubstituted linear, branched or cyclical alkyls, aryls, substituted aryls, substituted or unsubstituted linear, branched or cyclical heteroalkyls, substituted or unsubstituted heteroaryls;
Nu is a nucleophile;
m is 0, 1, 2, 3, 4, 5, or 6, and
Ar is a multi-substituted aromatic hydrocarbon or multi-substituted aromatic heterocycle.

Preferably, Nu of formulas (II) and (III) is selected from the group comprising primary, secondary and tertiary amine; thiol; carboxylic acid; hydroxylamine; hydrazine; and nitrogen containing heteroaryl.

Preferably, Ar of formulas (II) and (III) is selected from one of the following structures:

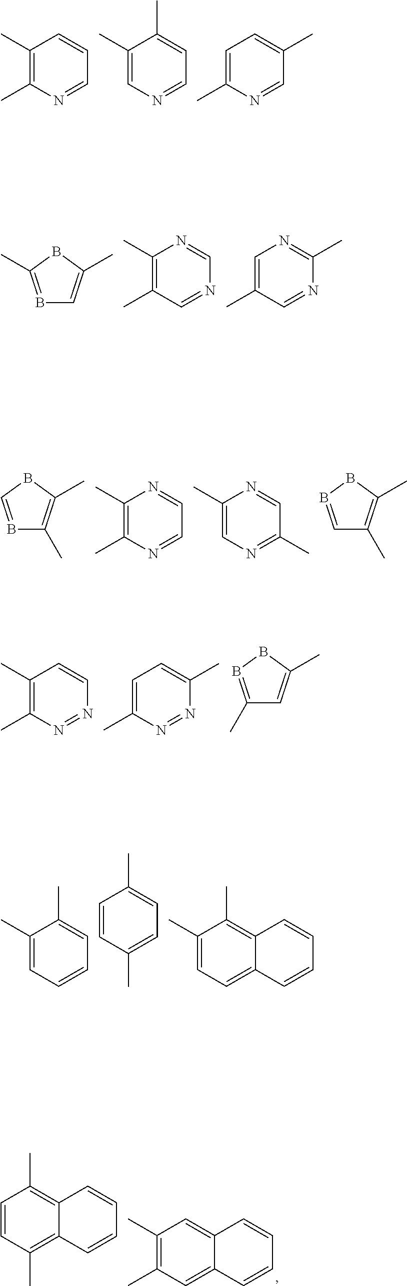

wherein each B is independently selected from O, S, N.

Preferably, R2, R3, R4, R5, R6, R7, R8 and W of formulas (II) and (III) are independently selected from hydrogen, methyl, ethyl, ethoxy, methoxy, and other C₁₋₆ linear, cyclical or branched alkyls and heteroalkyls.

Another suitable reversible prodrug linker moiety for amine-comprising drugs is described in WO-A 2006/136586. Accordingly, the following sub-structures selected from the general formulas (IV), (V) and (VI) are preferred embodiments for reversible prodrug linker-biologically active moiety conjugates:

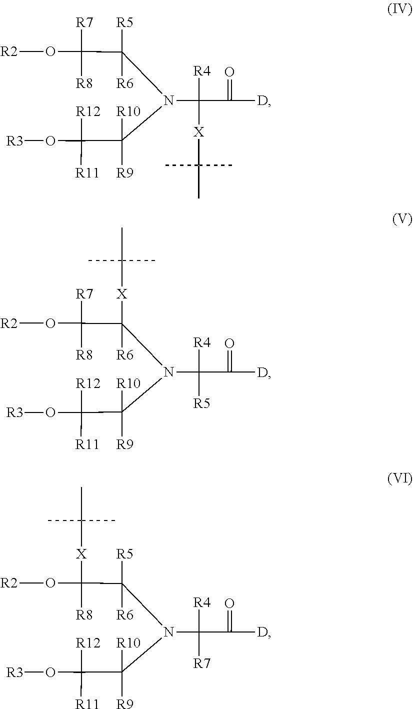

wherein the dashed line indicates attachment to the hydrogel or to a spacer moiety which is connected to the hydrogel, and wherein X, R2, R3, R4, R5, R6, R7, R8, R9, R10, R11, R12 and D of formulas (IV), (V) and (VI) have the following meaning:
D is an amine-comprising biologically active moiety;
X is a spacer R13-Y1;
Y1 is O, S, NR6, succinimide, maleimide, an unsaturated carbon-carbon bond, or any heteroatom-containing a free electron pair or Y1 is absent;
R2 and R3 are selected independently from hydrogen, acyl groups, and protecting groups for hydroxyl groups;
R4 to R12 are selected independently from hydrogen, substituted or non-substituted linear, branched or cyclical alkyl or heteroalkyl, aryls, substituted aryls, substituted or non-substituted heteroaryls, cyano, nitro, halogen, carboxy, and carboxamide; and
R13 is selected from substituted or non-substituted linear, branched or cyclical alkyl or heteroalkyl, aryls, substituted aryls, substituted or non-substituted heteroaryls.

Another suitable reversible prodrug linker moiety for primary amine- or secondary amine-comprising drugs is described in WO-A 2009/095479. Accordingly, a preferred hydrogel-linked prodrug is given by a prodrug conjugate D-L, wherein
-D is the primary amine- or secondary amine-comprising biologically active moiety; and
-L is a non-biologically active linker moiety -L¹ represented by formula (VII),

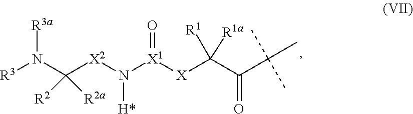

wherein the dashed line indicates the attachment to a primary or secondary amino group of an amine-containing biologically active moiety D by forming an amide bond; and wherein X, X¹, X², R¹, R^{1a}, R², R^{2a}, R³, and R^{3a} of formula (VII) have the following meaning:
X is C(R⁴R^{4a}); N(R⁴); O; C(R⁴R^{4a})—C(R⁵R^{5a}); C(R⁵R^{5a})—C(R⁴R^{4a});
C(R⁴R^{4a})—N(R⁶); N(R⁶)—C(R⁴R^{4a}); C(R⁴R^{4a})—O; or O—C(R⁴R^{4a});
X¹ is C; or S(O);
X² is C(R⁷, R^{7a}); or C(R⁷, R^{7a})—C(R¹, R^{8a}
R¹, R^{1a}, R², R^{2a}, R³, R^{3a}, R⁴, R^{4a}R⁵, R^{5a}, R⁶, R^{6a}, R^{7a}, R⁸, R^{8a} are independently selected from the group consisting of H; and C₁₋₄ alkyl; or
optionally, one or more of the pairs R^{1a}/R^{4a}, R^{1a}/R^{5a}, R^{4a}/R^{5a}, R^{4a}/R^{5a}, R^{7a}/R^{8a} form a chemical bond;
optionally, one or more of the pairs R¹/R^{1a}, R²/R^{2a}, R⁴/R^{4a}, R⁵/R^{5a}, R⁷/R^{7a}, R⁸/R^{8a} are joined together with the atom to which they are attached to form a C₃₋₇ cycloalkyl; or 4 to 7 membered heterocyclyl;
optionally, one or more of the pairs R¹/R⁴, R¹/R⁵, R¹/R⁶, R⁴/R⁵, R⁷/R⁸, R²/R³ are joined together with the atoms to which they are attached to form a ring A;
optionally, R³/R^{3a} are joined together with the nitrogen atom to which they are attached to form a 4 to 7 membered heterocycle;
A is selected from the group consisting of phenyl; naphthyl; indenyl; indanyl; tetralinyl; C₃₋₁₀ cycloalkyl; 4 to 7 membered heterocyclyl; and 9 to 11 membered heterobicyclyl; and
wherein L¹ is substituted with one group L²-Z and optionally further substituted, provided that the hydrogen marked with the asterisk in formula (VII) is not replaced by a substituent; and
   wherein
   L² is a single chemical bond or a spacer; and
   Z is the hydrogel of the hydrogel-linked prodrug.

Thus, the hydrogel is attached to any one of R¹, R^{1a}, R², R^{2a}, R³, R^{3a}, X, or X² of formula (VII), either directly (if L² is a single chemical bond) or through a spacer moiety (if L² is a spacer).

Optionally, L¹ in formula (VII) is further substituted, provided that the hydrogen marked with the asterisk in formula (VII) is not replaced by a substituent. Preferably, the one or more further optional substituents are independently selected from the group consisting of halogen, CN, COOR⁹, OR⁹, C(O)R⁹, C(O)N(R⁹R^{9a}), S(O)₂N(R⁹R^{9a}), S(O)N(R⁹R^{9a}), S(O)₂R⁹, S(O)R⁹, N(R⁹)S(O)₂N(R^{9a}R⁹), SR⁹, N(R⁹R^{9a}), NO₂, OC(O)R⁹, N(R⁹)C(O)R^{9a} N(R⁹)S(O)₂R^{9a}, N(R⁹)S(O)R^{9a}, N(R⁹)C(O)OR^{9a}, N(R⁹)C(O)N(R^{9a}R^{9b}), OC(O)N(R⁹R^{9a}) T, C₁₋₅₀ alkyl, C₂₋₅₀ alkenyl, and C₂₋₅₀ alkynyl,

wherein T, C₁₋₅₀ alkyl, C₂₋₅₀ alkenyl, and C₂₋₅₀ alkynyl are optionally substituted with one or more R¹⁰, which are the same or different, and wherein C₁₋₅₀ alkyl; C₂₋₅₀ alkenyl; and C₂₋₅₀ alkynyl are optionally interrupted by one or more groups selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹¹R^{11a});

T is selected from the group consisting of phenyl, naphthyl, indenyl, indanyl, tetralinyl, C₃₋₁₀ cycloalkyl, 4- to 7-membered heterocyclyl, and 9- to 11-membered heterobicyclyl, wherein T is optionally substituted with one or more R¹⁰, which are the same or different, R⁹, R^{9a}, R^{9b} are independently selected from the group consisting of H; T; and C₁₋₅₀ alkyl;

C₂₋₅₀ alkenyl; and C₂₋₅₀ alkynyl,

R¹⁰ is halogen, CN, oxo (═O), COOR¹², OR¹², C(O)R¹², C(O)N(R¹²R^{12a}) S(O)₂N(R¹²R^{12a}), S(O)N(R¹²R^{12a}) S(O)₂R¹², S(O)R¹, N(R¹²)S(O)₂N(R^{12a}R^{12b}), SR¹², N(R¹²R^{12a}), NO₂, OC(O)R¹², N(R¹²)C(O)R^{12a}, N(R¹²)S(O)₂R^{12a}, N(R¹²)S(O)R^{12a} N(R¹²)C(O)OR^{12a}, N(R¹²)C(O)N(R^{12a}R^{12b}), OC(O)N(R¹²R^{12a}), or C₁₋₆ alkyl, wherein C₁₋₆ alkyl is optionally substituted with one or more halogen, which are the same or different, R¹¹, R^{11a}, R¹², R¹², R^{12b} are independently selected from the group consisting of H; or C₁₋₆ alkyl, wherein C₁₋₆ alkyl is optionally substituted with one or more halogen, which are the same or different.

The term “interrupted” means that between two carbons a group is inserted or at the end of the carbon chain between the carbon and hydrogen.

Preferred moieties L¹ according to formula (VII) are selected from the group consisting of:

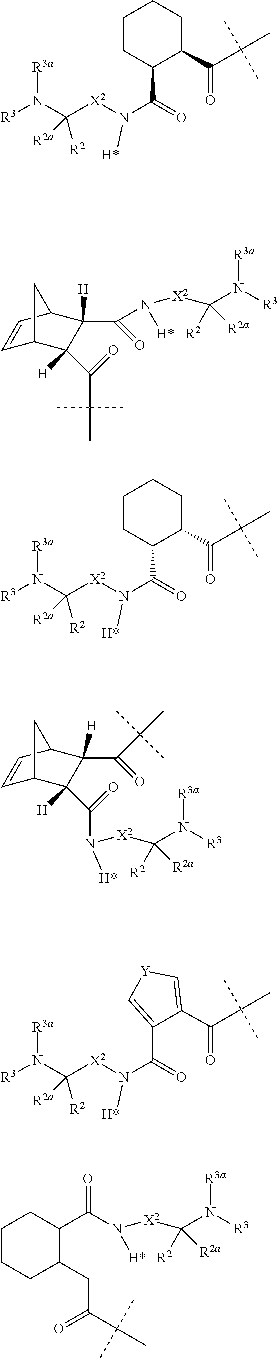

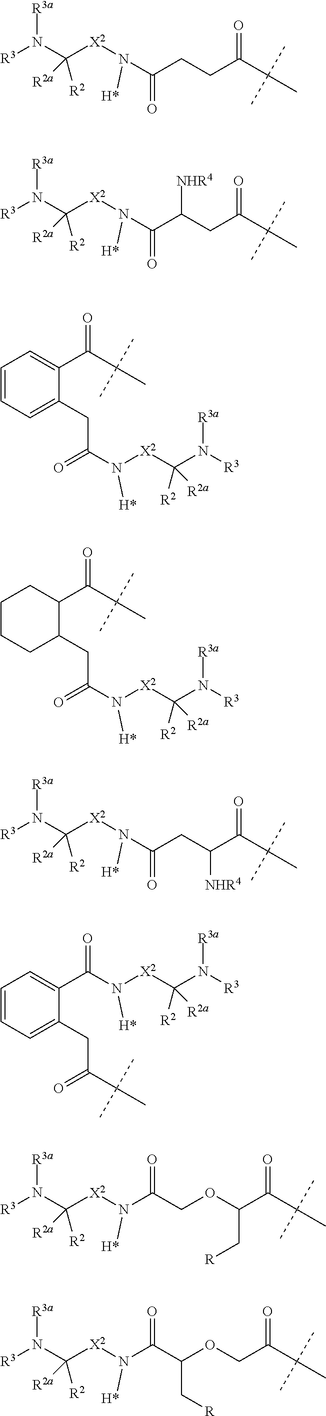

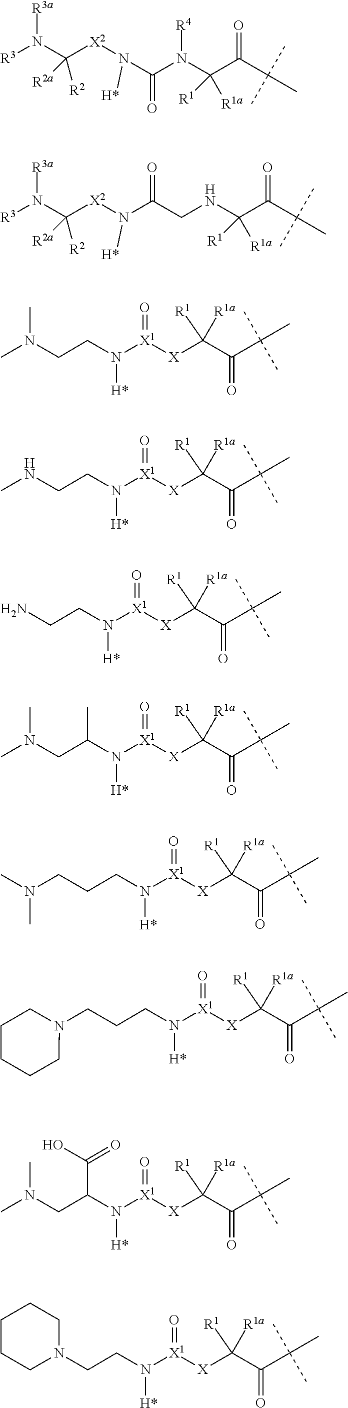

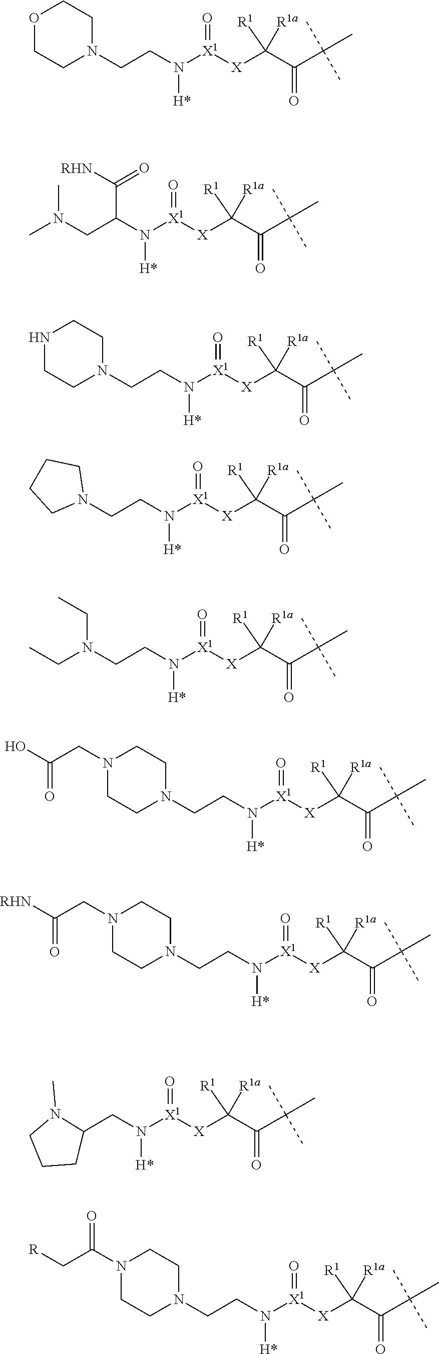

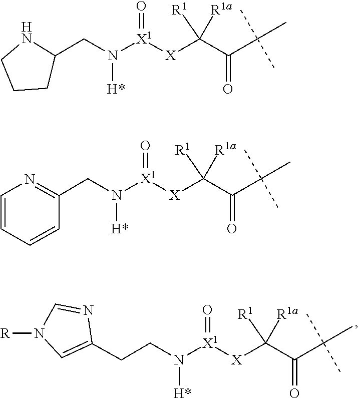

wherein

dashed lines indicate attachment to D of formula (VII);
R is H or C₁₋₄ alkyl;
Y is NH, O or S; and
R¹, R^{1a}, R², R^{2a}, R³, R^{3a}, R⁴, X, X¹, X² have the meaning as indicated in formula (VII).

Even more preferred moieties L¹ of formula (VII) are selected from the group consisting of:

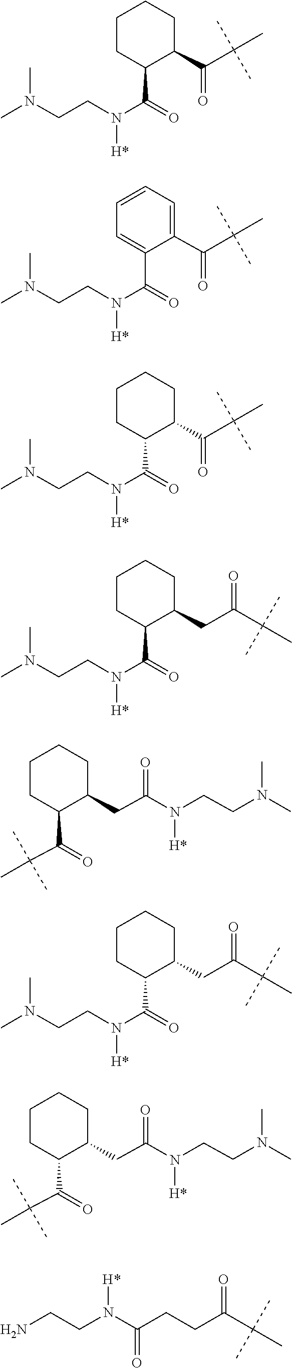

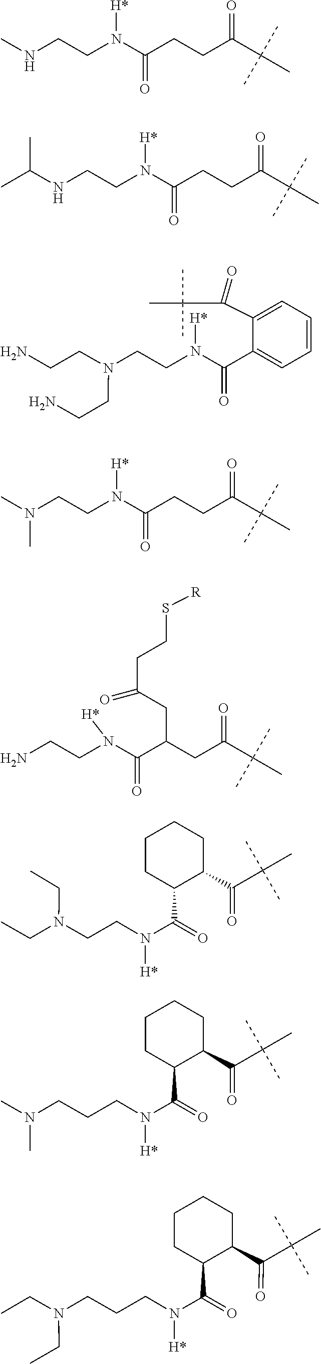

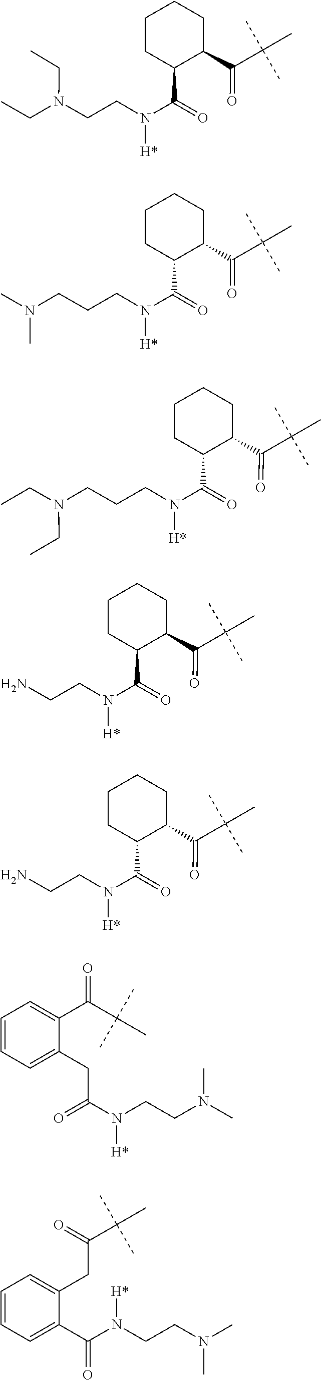

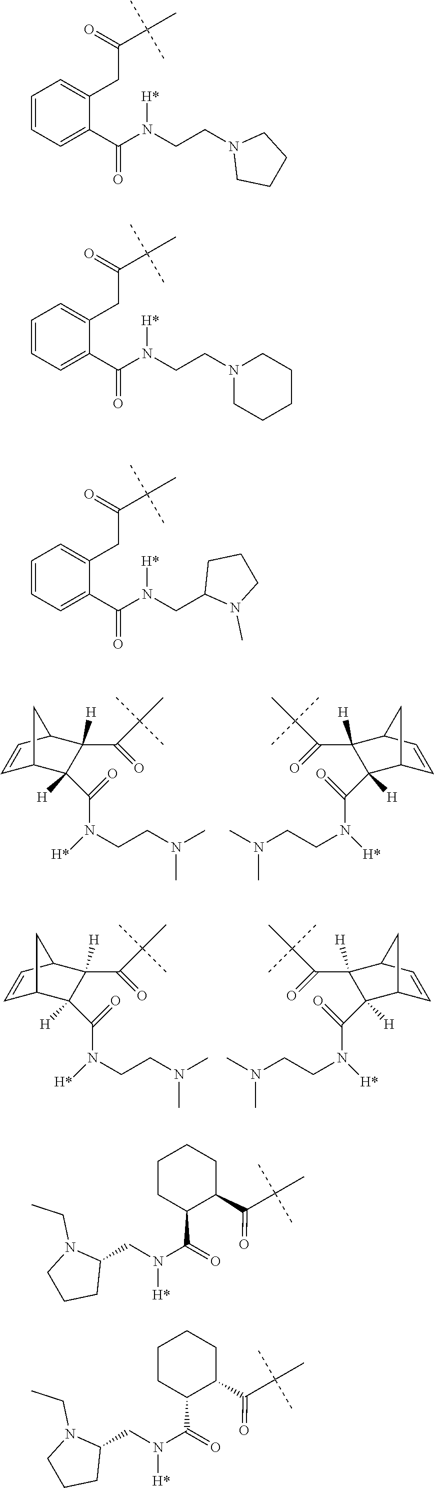

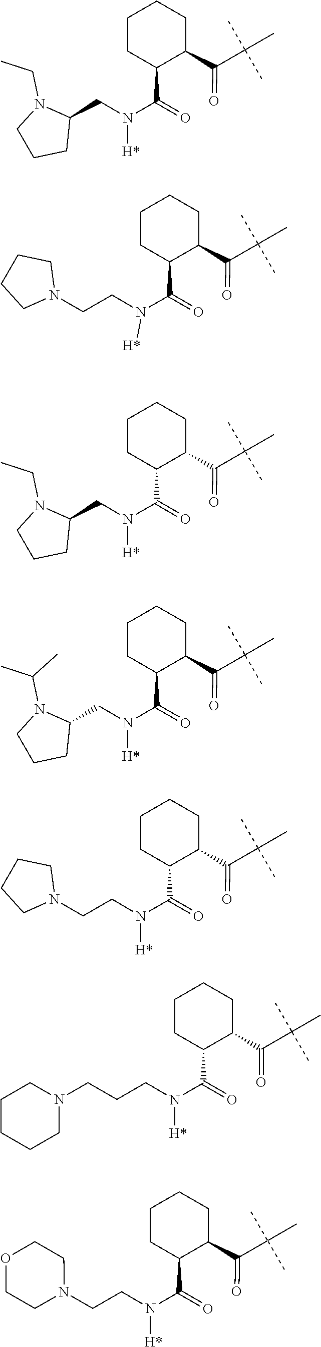

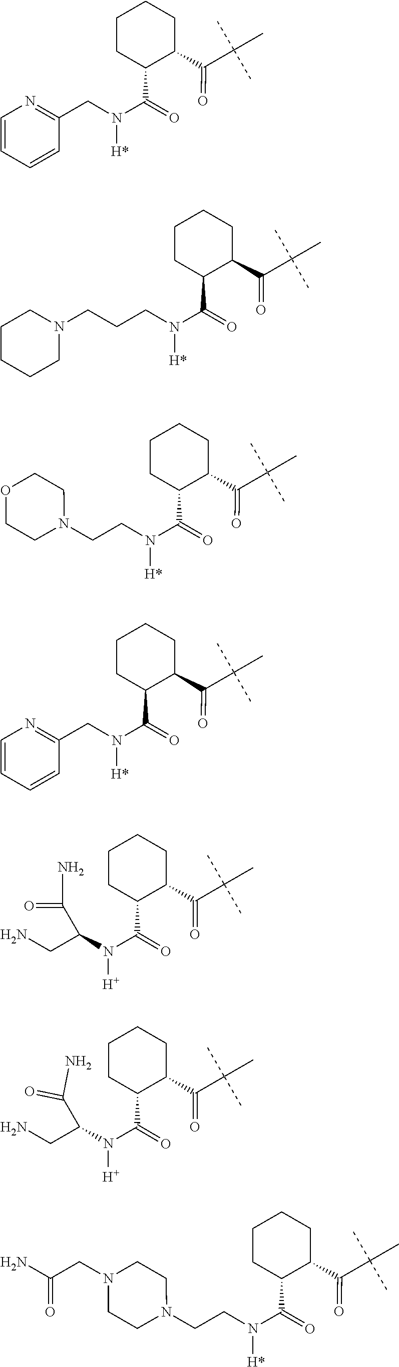

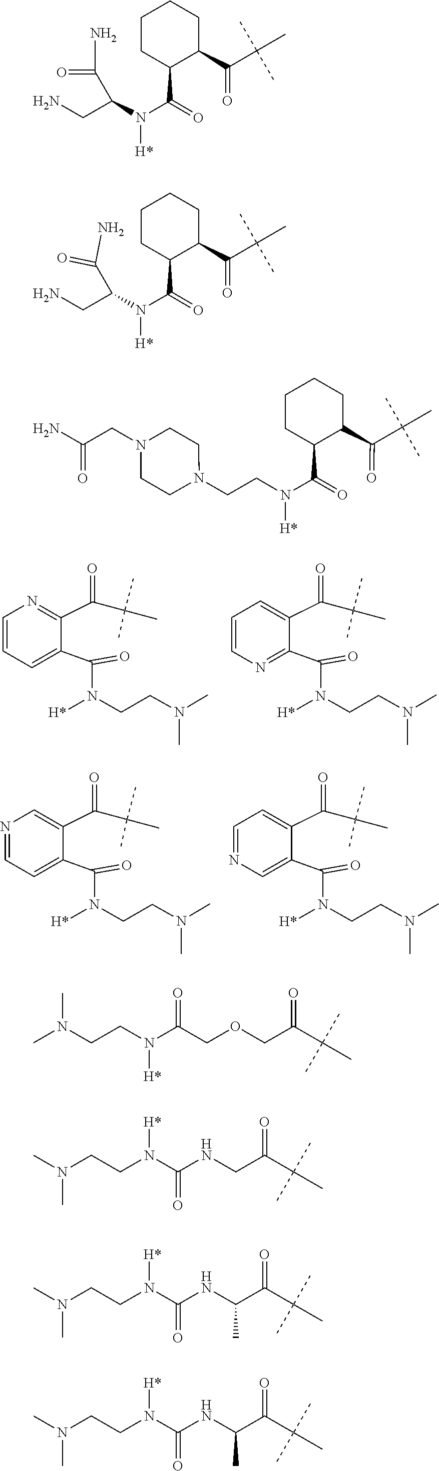

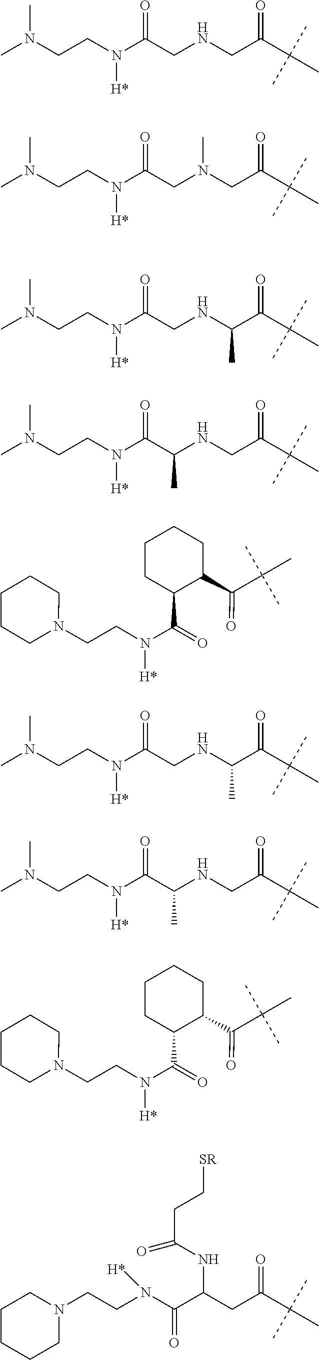

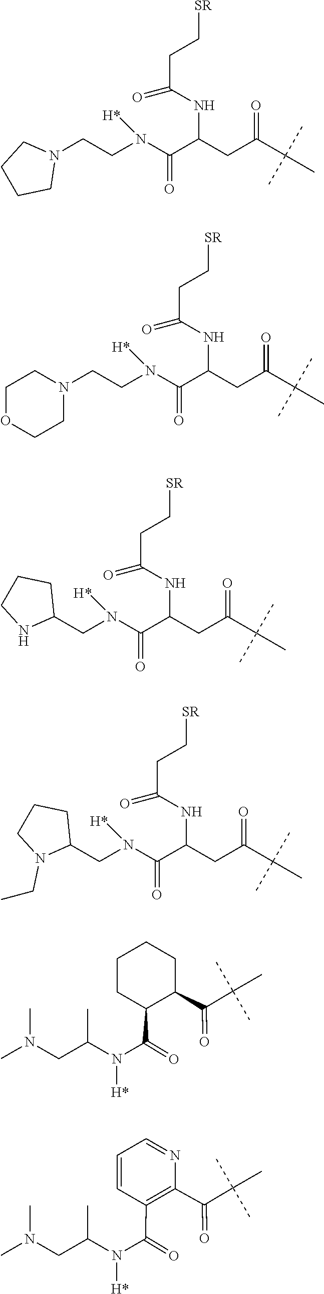

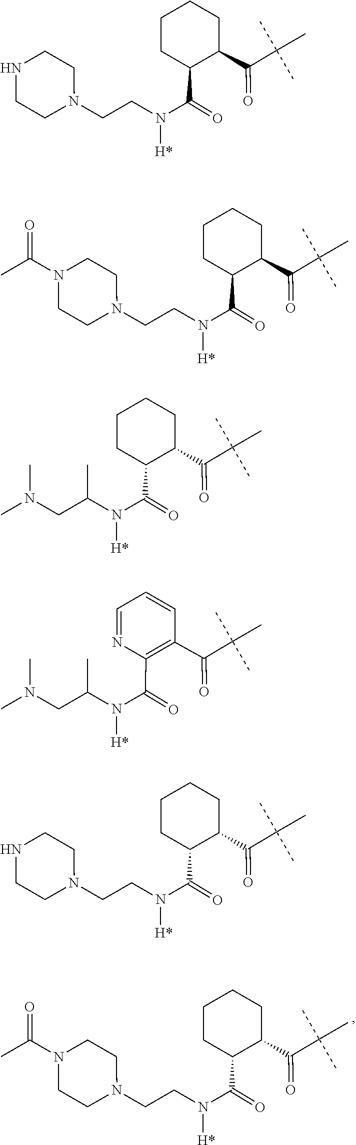

wherein
dashed lines indicate attachment to D of formula (VII), and
R is H or C₁₋₄ alkyl.

Another preferred hydrogel-linked prodrug is given by a conjugate D-L, wherein
-D is the biologically active moiety; and
-L is a non-biologically active linker moiety -L¹ represented by formula (VIII),

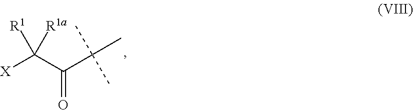

wherein the dashed line indicates attachment to a primary amine- or secondary amine-comprising biologically active moiety D by forming an amide bond; and wherein X, R¹, and R^{1a} of formula (VIII) have the following meaning:
X is H or C₁₋₅₀ alkyl, optionally interrupted by one or more groups selected from —NH—, —C(C₁₋₄ alkyl)-, —O—, —C(O)— or —C(O)NH—;
R¹ and R^{1a} are independently selected from the group consisting of H and C₁-C₄ alkyl;
wherein L¹ is substituted with one group L²-Z and optionally further substituted; and wherein
   L² is a single chemical bond or a spacer; and
   Z is the hydrogel of the hydrogel-linked prodrug.

Thus, the hydrogel is attached to any one of R¹, R^{1a} or X of formula (VIII), either directly (if L² is a single chemical bond) or through a spacer moiety (if L² is a spacer).

Optionally, the sub-structure of formula (VIII) is further substituted.

More preferably, L¹ of formula (VIII) comprises one of the fragments of formulas (VIIIb) or (VIIIc), wherein the dashed line marked with an asterisk indicates attachment to D by forming an amide bond with the aromatic amino group of D and the unmarked dashed line indicates attachment to the rest of L1 of formula (VIII) and wherein the structures of formulas (VIIIb) and (VIIIc) are optionally further substituted:

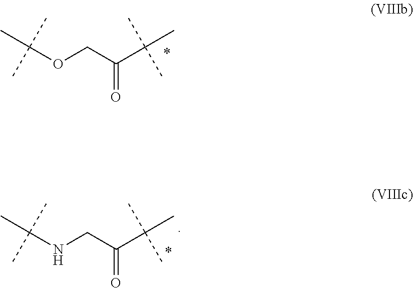

More preferably, L¹ of formula (VIII) comprises one of the fragments of formulas (VIIIba), (VIIIca), or (VIIIcb), wherein the dashed line marked with an asterisk indicates attachment to D of formula (VIII) by forming an amide bond with the aromatic amino group of D and the unmarked dashed line indicates attachment to the rest of L of formula (VIII):

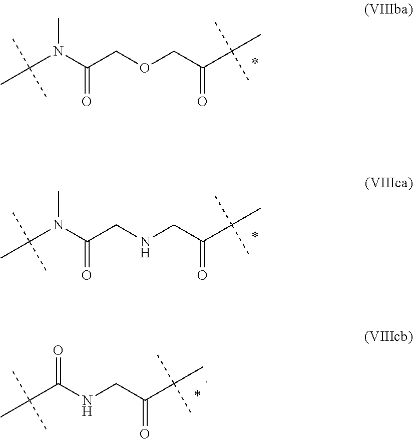

Another suitable reversible prodrug linker moiety for aromatic amine-comprising drugs is described in WO-A 2011/012721. Accordingly, a preferred hydrogel-linked prodrug is given by a conjugate D-L, wherein
-D is the biologically active moiety; and
-L is a non-biologically active linker moiety -L¹ represented by formula (IX),

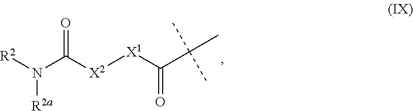

wherein the dashed line indicates the attachment to an aromatic amine group of an aromatic amine-containing biologically active moiety D by forming an amide bond; and wherein X¹, X², R² and R^{2a} of formula (IX) have the following meaning:
X¹ is C(R¹R^{1a}) or a cyclic fragment selected from C₃₋₇ cycloalkyl, 4- to 7-membered heterocyclyl, phenyl, naphthyl, indenyl, indanyl, tetralinyl, and 9- to 11-membered heterobicyclyl,
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, and O—C(R³R^{3a}),
wherein in case X¹ is a cyclic fragment, X² is a chemical bond, C(R³R^{3a}) N(R³) or O,
optionally, in case X¹ is a cyclic fragment and X² is C(R³R^{3a}), the order of the X¹ fragment and the X² fragment shown in formula (IX) may be changed,
R¹, R³ and R⁴ are independently selected from the group consisting of H, C₁₋₄ alkyl and —N(R⁵R^{5a})
R^{1a}, R², R^{2a}, R^{3a}, R^{4a} and R^{5a} are independently selected from the group consisting of H, and C₁₋₄ alkyl,
optionally, one of the pairs R^{2a}/R², R^{2a}/R^{3a}, R^{2a}/R^{4a} are joined to form a 4- to 7-membered at least partially saturated heterocycle,
R⁵ is C(O)R⁶,
R⁶ is C₁₋₄ alkyl,
optionally, one of the pairs R^{1a}/R^{4a}, R^{3a}/R^{4a} or R^{1a}/R^{3a} form a chemical bond; and
wherein L¹ is substituted with one group L²-Z and optionally further substituted; and wherein
   L² is a single chemical bond or a spacer; and
   Z is the hydrogel of the hydrogel-linked prodrug.

Thus, the hydrogel is attached to any one of X¹, X², R¹, R^{1a}, R², R^{2a}, R³, R^{3a}, R⁴, R⁵, R^{5a} or R⁶ of formula (IX), either directly (if L² is a single chemical bond) or through a spacer moiety (if L² is a spacer).

More preferably, the moiety L¹ according to formula (IX) is selected from the following formulas:

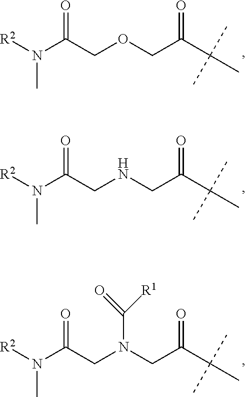

wherein the dashed line indicates attachment to the biologically active moiety D, and
R¹ and R² are used as defined in formula (IX).

Preferably, R^{1a}, R², R^{2a}, R^{3a}, R^{4a} and R^{5a} of formula (IX) are independently selected from the group consisting of H, and C₁₋₄ alkyl.

Another suitable reversible prodrug linker moiety for aromatic amine-comprising drugs is described in WO 2011/012722. Accordingly, a preferred linker structure for the hydrogel-linked prodrug is given by a conjugate D-L, wherein
-D is the biologically active moiety; and
-L is a non-biologically active linker moiety -L¹ represented by formula (X),

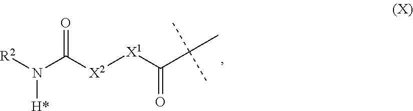

wherein the dashed line indicates attachment to an aromatic amine group of an aromatic amine-containing biologically active moiety D; and
wherein X¹, X², and R² of formula (X) have the following meaning:
X¹ is C(R¹R^{1a}) or a cyclic fragment selected from C₃₋₇ cycloalkyl, 4 to 7 membered heterocyclyl, phenyl, naphthyl, indenyl, indanyl, tetralinyl, and 9 to 11 membered heterobicyclyl;
wherein in case X¹ is a cyclic fragment, said cyclic fragment is incorporated via two adjacent ring atoms and the ring atom of X¹, which is adjacent to the carbon atom of the amide bond, is also a carbon atom;
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, and O—C(R³R^{3a});
wherein in case X¹ is a cyclic fragment, X² is a chemical bond, C(R³R^{3a}) N(R³) or O;
optionally, in case X¹ is a cyclic fragment and X² is C(R³R^{3a}), the order of the X¹ fragment and the X² fragment shown in formula (X) may be changed and the cyclic fragment is incorporated into the sub-structure of formula (X) via two adjacent ring atoms;
R¹, R³ and R⁴ are independently selected from the group consisting of H, C₁₋₄ alkyl and —N(R⁵R^{5a});
R^{1a}, R², R^{3a}, R^{4a} and R^{5a} are independently selected from the group consisting of H, and C₁₋₄ alkyl;
R⁵ is C(O)R⁶;
R⁶ is C₁₋₄ alkyl;
optionally, one of the pairs R^{1a}/R^{4a}, R^{3a}/R^{4a} or R^{1a}/R^{3a} form a chemical bond, provided that the hydrogen marked with the asterisk in formula (X) is not replaced;
wherein L¹ is substituted with one group L²-Z and optionally further substituted, provided that the hydrogen marked with the asterisk in formula (X) is not replaced; and wherein
   L² is a single chemical bond or a spacer; and
   Z is the hydrogel of the hydrogel-linked prodrug.

Thus, the hydrogel is attached to any one of X¹, X², R¹, R^{1a}, R², R³, R^{3a}, R⁴, R⁵, R^{5a} or R⁶ of formula (X), either directly (if L² is a single chemical bond) or through a spacer moiety (if L² is a spacer).

More preferably, the moiety L¹ of formula (X) is selected from the group consisting of formulas (i) through (xxix):

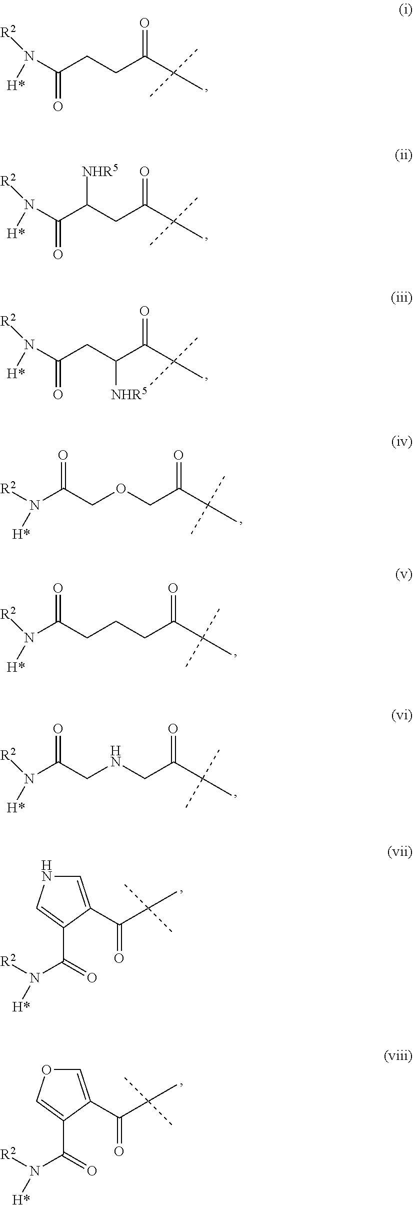

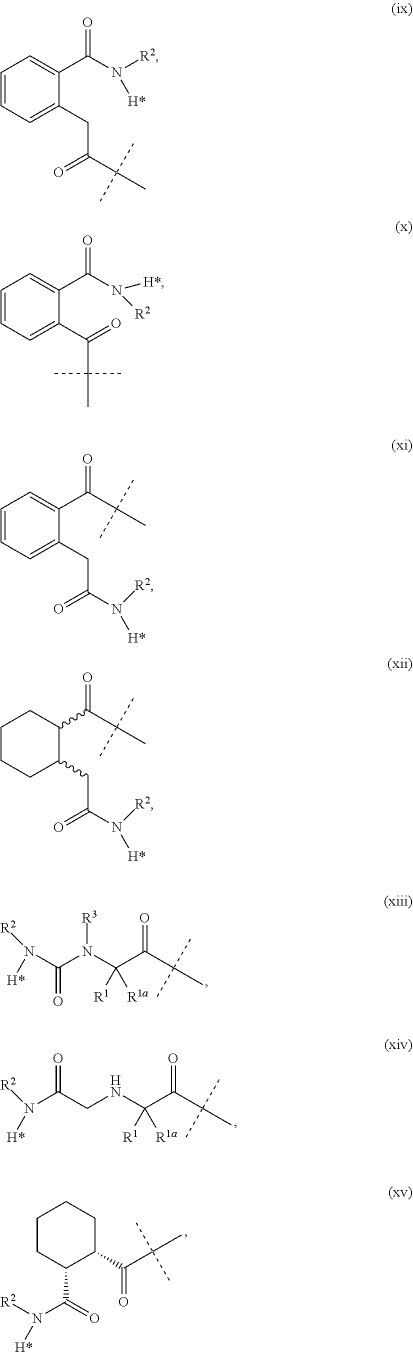

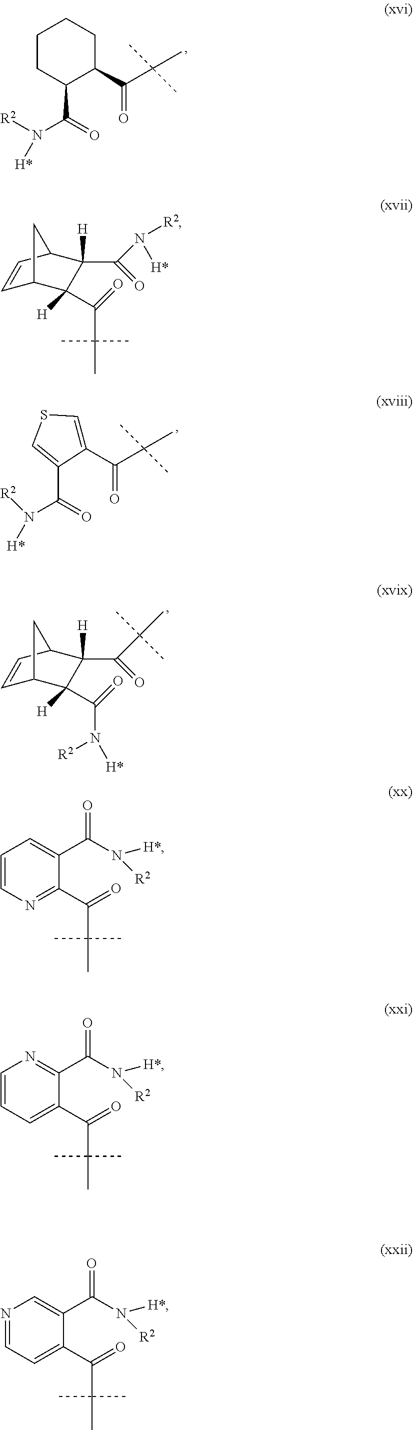

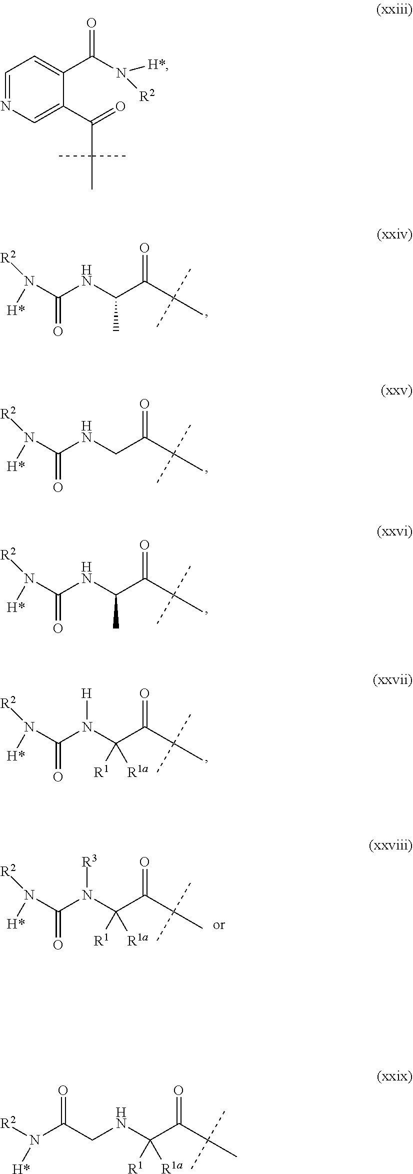

wherein the dashed line indicates attachment to D, and
R¹, R^{1a}, R², R³, and R⁵ are used as defined in formula (X).

The amino substituent of the aromatic fragment of D forms together with the carbonyl-fragment (—C(O)—) on the right hand side of L¹ (as depicted in formula (X)) an amide bond between L¹ and D. By consequence, D and L¹ of formula (X) are connected (chemically bound) by an amide fragment of the general structure Y¹—C(O)—N(R)—Y². Y¹ indicates the remaining parts of the sub-structure of formula (X) and Y² indicates the aromatic fragment of D. R is a substituent, such as C₁₋₄ alkyl or preferably hydrogen.

As indicated above, X¹ of formula (X) may also be a cyclic fragment such as C₃₋₇ cycloalkyl, phenyl or indanyl. In case X¹ is such a cyclic fragment, the respective cyclic fragment is incorporated into L¹ of formula (X) via two adjacent ring atoms (of said cyclic fragment). For example, if X¹ is phenyl, the phenyl fragment of L¹ is bound to X² of L¹ via a first (phenyl) ring atom being in α-position (adjacent) to a second (phenyl) ring atom, which itself is bound to the carbon atom of the carbonyl-fragment on the right hand side of L¹ according to formula (X), i.e. the carbonyl fragment which together with the aromatic amino group of D forms an amide bond.

Preferably, L¹ of formula (X) is defined as follows:
X¹ is C(R¹R^{1a}), cyclohexyl, phenyl, pyridinyl, norbonenyl, furanyl, pyrrolyl or thienyl,
wherein in case X¹ is a cyclic fragment, said cyclic fragment is incorporated into L¹ of formula (X) via two adjacent ring atoms;
X² is a chemical bond or selected from C(R³R^{3a}), N(R³), O, C(R³R^{3a})—O or C(R³R^{3a})—C(R⁴R^{4a}).
R¹, R³ and R⁴ are independently selected from H, C₁₋₄ alkyl and —N(R⁵R^{5a});
R^{1a}, R^{3a}, R^{4a} and R^{5a} are independently selected from H and C₁₋₄ alkyl;
R² is C₁₋₄ alkyl;
R⁵ is C(O)R⁶;
R⁶ is C₁₋₄ alkyl;
More preferably, L¹ of formula (X) is selected from the following formulas (i) to (xxix):

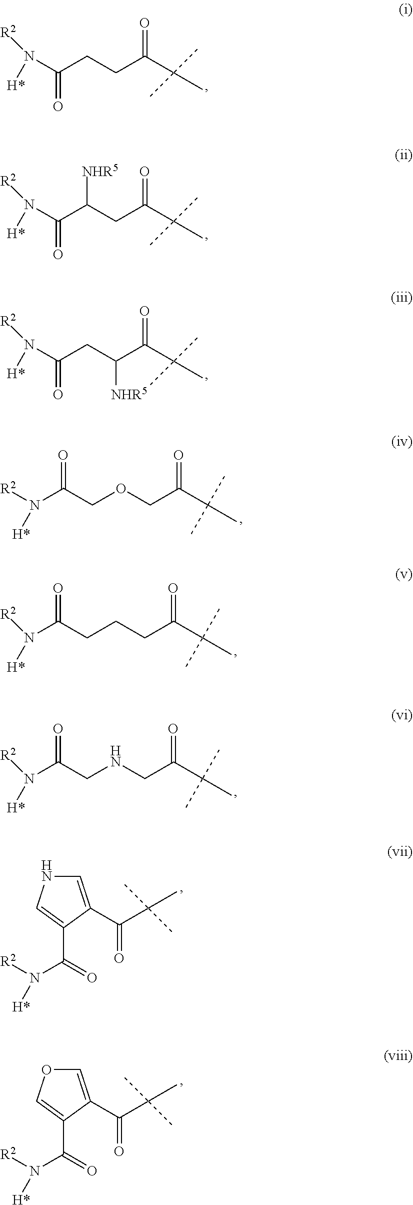

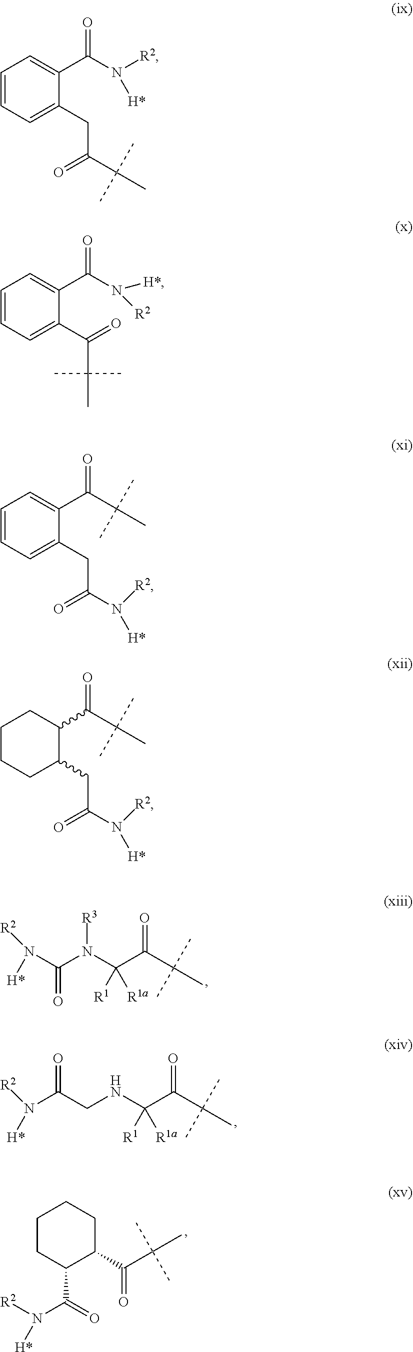

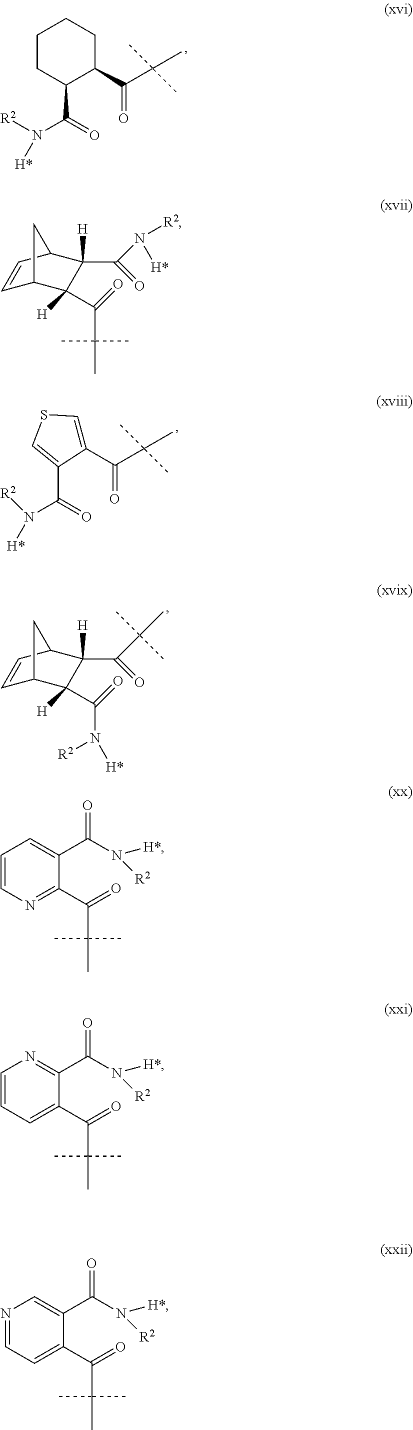

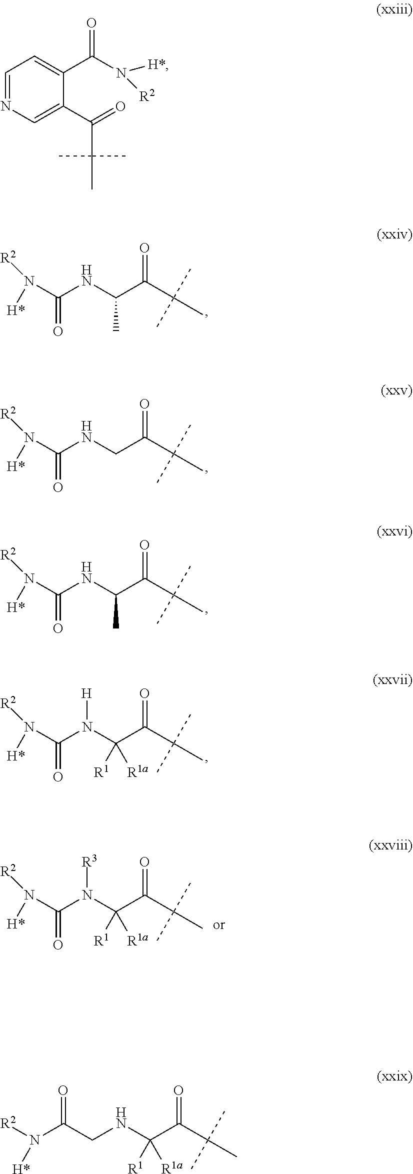

wherein the dashed line indicates attachment to D,
R⁵ is C(O)R⁶, and
R¹, R^{1a}, R², R³ and R⁶ are independently from each other C₁₋₄ alkyl.

Another suitable reversible prodrug linker moiety for hydroxyl-comprising drugs is described in WO 2011/012721. Accordingly, a preferred hydrogel-linked prodrug is given by formula (XI):

D-O—Z⁰ (XI),
wherein,
D is a hydroxyl-comprising biologically active moiety comprising 0 of formula (XI) which is coupled to the moiety Z⁰ through said oxygen of the hydroxyl group; and wherein Z⁰ of formula (XI) has the following meaning:
Z⁰ is C(O)—X⁰—Z¹; C(O)O—X⁰—Z¹; S(O)₂—X⁰—Z¹; C(S)—X⁰—Z¹; S(O)₂O—X⁰—Z¹; S(O)₂N(R¹)—X⁰—Z¹; CH(OR¹)—X⁰—Z¹; C(OR¹)(OR²)—X⁰—Z¹; C(O)N(R¹)—X⁰—Z¹; P(═O)(OH)O—X⁰—Z¹; P(═O)(OR¹)O—X⁰—Z¹; P(═O)(SH)O—X⁰—Z¹; P(═O)(SR¹)O—X⁰—Z¹; P(═O)(OR¹)—X⁰—Z¹; P(═S)(OH)O—X⁰—Z¹; P(═S)(OR¹)O—X⁰—Z¹; P(═S)(OH)N(R¹)—X⁰—Z¹; P(═S)(OR¹)N(R²)—X⁰—Z¹; P(═O)(OH)N(R¹)—X⁰—Z¹; or P(═O)(OR)N(R²)—X⁰—Z¹;
R¹, R² are independently selected from the group consisting of C₁₋₆ alkyl; or R¹, R² jointly form a C₁₋₄ alkylene bridging group;
X⁰ is (X^{0A})ₘ₁—(X^{0B})ₘ₂;
m1 and m2 are independently 0 or 1;
X^{0A} is T⁰;
X^{0B} is a branched or unbranched C₁₋₁₀ alkylene group which is unsubstituted or substituted with one or more R³, which are the same or different;
R³ is halogen; CN; C(O)R⁴; C(O)OR⁴; OR⁴; C(O)R⁴; C(O)N(R⁴R^{4a});
S(O)₂N(R⁴R^{4a}); S(O)N(R⁴R^{4a}); S(O)₂R⁴; S(O)R⁴; N(R⁴)S(O)₂N(R^{4a}R^{4b}); SR⁴;
N(R⁴R^{4a}); NO₂; OC(O)R⁴; N(R⁴)C(O)R^{4a}; N(R⁴)SO₂R^{4a}; N(R⁴)S(O)R^{4a};
N(R⁴)C(O)N(R^{4a}R^{4b}); N(R⁴)C(O)OR^{4a}; OC(O)N(R⁴R^{4a}); or T⁰;
R⁴, R^{4a}, R⁴ are independently selected from the group consisting of H; T⁰; C₁₋₄ alkyl; C₂₋₄ alkenyl; and C₂₋₄ alkynyl, wherein C₁₋₄ alkyl; C₂₋₄ alkenyl; and C₂₋₄ alkynyl are optionally substituted with one or more R⁵, which are the same of different;
R⁵ is halogen; CN; C(O)R⁶; C(O)OR⁶; OR⁶; C(O)R⁶; C(O)N(R⁶R^{6a}); S(O)₂N(R⁶R^{6a}); S(O)N(R⁶R^{6a}); S(O)₂R⁶; S(O)R⁶; N(R⁶)S(O)₂N(R^{6a}R⁶); SR⁶; N(R⁶R^{6a}); NO₂; OC(O)R⁶; N(R⁶)C(O)R^{6a}; N(R⁶)SO₂R^{6a}; N(R⁶)S(O)R^{6a}; N(R⁶)C(O)N(R^{6a}R^{6b}); N(R⁶)C(O)OR^{6a}; OC(O)N(R⁶R^{6a});
R⁶, R^{6a}, R^{6b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same of different;
T⁰ is phenyl; naphthyl; azulenyl; indenyl; indanyl; C₃₋₇ cycloalkyl; 3 to 7 membered heterocyclyl; or 8 to 11 membered heterobicyclyl, wherein T⁰, is optionally substituted with one or more R⁷, which are the same or different;
R⁷ is halogen; CN; COOR⁸; OR⁸; C(O)R⁸; C(O)N(R⁸R^{8a}); S(O)₂N(R⁸R^{8a}); S(O)N(R¹R^{1a}); S(O)₂R⁸; S(O)R⁸; N(R⁸)S(O)₂N(R^{8a}R^{8a}); SR⁸; N(R¹R^{1a}); NO₂; OC(O)R⁸; N(R⁸$)C(O)R^{8a}; N(R⁸$)S(O)₂R^{8a}; N(R⁸$)S(O)R^{8a}; N(R⁸)C(O)OR^{85a}; N(R⁸)C(O)N(R^{8a}R⁸); OC(O)N(R⁸R^{8a}); oxo (═O), where the ring is at least partially saturated; C₁_alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R⁹, which are the same or different;
R⁸, R^{8a}, R^{8b} are independently selected from the group consisting of H; C₁₋₆ alkyl;
C₂₋₆ alkenyl; and C₂₋₆ alkynyl, wherein C₁₋₆ alkyl; C₂₋₆ alkenyl; and C₂₋₆ alkynyl are optionally substituted with one or more R¹⁰, which are the same of different;
R⁹, R¹⁰ are independently selected from the group consisting of halogen; CN; C(O)R¹¹; C(O)OR¹¹; OR¹¹; C(O)R¹¹; C(O)N(R¹¹R^{11a}); S(O)₂N(R¹¹R^{11a}), S(O)N(R¹¹R^{11a}); S(O)₂R¹¹; S(O)Ra; N(R¹¹)S(O)₂N(R^{11a}R^{11b}); SR¹¹; N(R¹¹R^{11a}); NO₂; OC(O)R¹¹; N(R¹¹)C(O)R^{11a}; N(R¹¹)SO₂R^{11a}; N(R¹¹)S(O)R^{11a}; N(R¹¹)C(O)N(R^{11a}R^{11b}); N(R¹¹)C(O)OR^{11a}; and OC(O)N(R¹¹R^{11a});
R¹¹, R^{11a}, R^{11b} are independently selected from the group consisting of H; C₁₋₄ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same of different;
Z¹ is the hydrogel of the hydrogel-linked prodrug, which is covalently attached to X⁰.

Preferably, Z⁰ is C(O)—X⁰—Z¹; C(O)O—X⁰—Z¹; or S(O)₂—X⁰—Z¹. More preferably, Z⁰ is C(O)—X⁰—Z¹; or C(O)O—X⁰—Z¹. Even more preferably, Z⁰ is C(O)—X⁰—Z¹.

Preferably, X⁰ is unsubstituted.

Preferably, m1 is 0 and m2 is 1.

Preferably, X⁰—Z⁰ is C(R¹R²)CH₂—Z⁰, wherein R¹, R² are independently selected from the group consisting of H and C₁₋₄ alkyl, provided that at least one of R¹, R² is other than H; or (CH₂)ₙ—Z⁰, wherein n is 3, 4, 5, 6, 7 or 8.

Preferably, Z¹ is covalently attached to X⁰ via amide group.

Another suitable reversible prodrug linker moiety for aromatic hydroxyl-comprising drugs is described in WO-A 2011/089214. Accordingly, a preferred hydrogel-linked prodrug is given by a conjugate D-L, wherein
D is a biologically active moiety containing an aromatic hydroxyl group; and
L is a non-biologically active linker containing
i) a moiety L¹ represented by formula (XII),

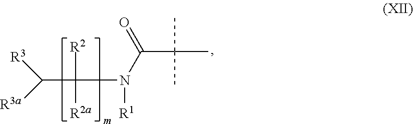

wherein the dashed line indicates the attachment of L¹ to the aromatic hydroxyl group of D by forming a carbamate group and R¹, R², R^{2a}, R³, R^{3a} and m of formula (XII) have the following meaning:
R¹ is selected from the group consisting of C₁₋₄ alkyl, heteroalkyl, C₃₋₇ cycloalkyl, and

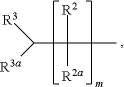

each R², each R^{2a}, R³, R^{3a} are independently selected from hydrogen, substituted or non-substituted linear, branched or cyclic C₁₋₄ alkyl or heteroalkyl,
m is 2, 3 or 4.
ii) a moiety L², which is a chemical bond or a spacer, and L² is bound to the hydrogel of the hydrogel-linked prodrug;
   wherein L¹ is substituted with one L² moiety.

Optionally, L is further substituted.

Thus, the hydrogel is attached to any one of R¹, R², R^{2a}, R³ or R^{3a} of formula (XII), either directly (if L² is a single chemical bond) or through a spacer moiety (if L² is a spacer).

Another suitable reversible prodrug linker moiety for aliphatic amine-comprising drugs is described in WO-A 2011/089216. Accordingly, a preferred hydrogel-linked prodrug is given by a conjugate D-L,
wherein
D is an aliphatic amine-comprising biologically active moiety; and
L is a non-biologically active linker containing
i) a moiety L¹ represented by formula (XIII),

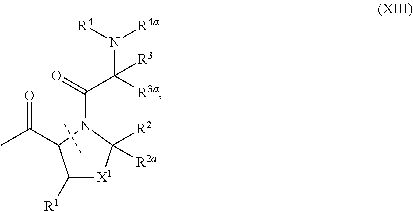

wherein the dashed line indicates the attachment of L¹ to an aliphatic amino group of D by forming an amide bond and wherein X¹, R¹, R², R^{2a}, R³, R^{3a}, R⁴ and R^{4a} of formula (XIII) have the following meaning:
X¹ is selected from O, S and CH—R^{1a}.
R¹ and R^{1a} are independently selected from H, OH, and CH₃;
R², R^{2a}, R⁴ and R^{4a} are independently selected from H and C₁₋₄ alkyl;
R³, R^{3a} are independently selected from H, C₁₋₄ alkyl, and R⁵
R⁵ is selected from

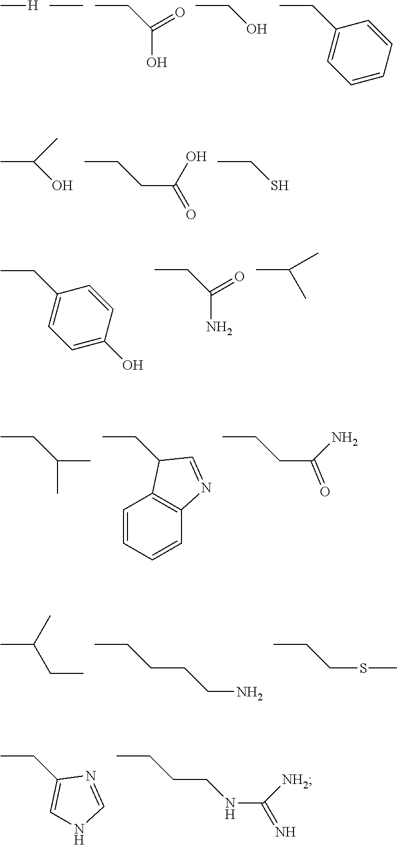

ii) a moiety L², which is a chemical bond or a spacer, and L² is bound to Z, which is the hydrogel of the hydrogel-linked prodrug;
   wherein L is substituted with one L² moiety,
   optionally, L is further substituted.

Thus, the hydrogel is attached to any one of X¹, R¹, R², R^{2a}, R³, R^{3a}, R⁴ or R^{4a} of formula (XIII), either directly (if L² is a single chemical bond) or through a spacer moiety (if L² is a spacer).

Preferably, one of the pair R³/R^{3a} of formula (XIII) is H and the other one is selected from R⁵.

Preferably, one of R⁴/R^{4a} of formula (XIII) is H.

Optionally, one or more of the pairs R³/R^{3a}, R⁴/R^{4a}, R³/R⁴ of formula (XIII) may independently form one or more cyclic fragments selected from C₃₋₇ cycloalkyl, 4 to 7 membered heterocyclyl, or 9 to 11 membered heterobicyclyl.

Optionally, R³, R^{3a}, R⁴ and R^{4a} of formula (XIII) are further substituted. Suitable substituents are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4- to 7-membered heterocycle) or halogen moieties.

Another suitable reversible prodrug linker moiety for aromatic amine-comprising drugs is described in WO-A 2011/089215. Accordingly, a preferred hydrogel-linked prodrug is given by a conjugate D-L,
wherein
D is an aromatic amine-comprising biologically active moiety; and
L is a non-biologically active linker containing
i) a moiety L¹ represented by formula (XIV),

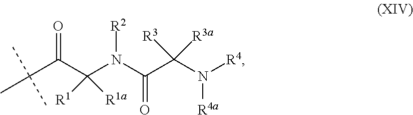

wherein the dashed line indicates the attachment of L¹ to an aromatic amino group of D by forming an amide bond and wherein R¹, R^{1a}, R², R³, R^{3a}, R⁴ and R^{4a} of formula (XIV) have the following meaning:
R¹, R^{1a}, R², R³, R^{3a}, R⁴ and R^{4a} are independently selected from H and C₁₋₄ alkyl,
optionally, any two of R¹, R^{1a}, R², R³, R^{3a}, R⁴ and R^{4a} may independently form one or more cyclic fragments selected from C₃₋₇ cycloalkyl, 4 to 7 membered heterocyclyl, phenyl, naphthyl, indenyl, indanyl, tetralinyl, or 9 to 11 membered heterobicyclyl,
optionally, R¹, R^{1a}, R², R³, R^{3a}, R⁴ and R^{4a} are further substituted; suitable substituents are alkyl, alkene, alkine, aryl, heteroalkyl, heteroalkene, heteroalkine, heteroaryl or halogen moieties.
ii) a moiety L², which is a chemical bond or a spacer, and L² is bound to Z, which is the hydrogel of the hydrogel-linked prodrug;
   wherein L¹ is substituted with one moiety L²,
   optionally, L is further substituted.

Suitable substituents are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Thus, the hydrogel is attached to any one of R¹, R^{1a}, R², R³, R^{3a}, R⁴ or R^{4a} of formula (XIV), either directly (if L² is a single chemical bond) or through a spacer moiety (if L² is a spacer).

Preferably, one of R⁴ or R^{4a} of formula (XIV) is H.

Another suitable reversible prodrug linker moiety is described in U.S. Pat. No. 7,585,837. Accordingly, a preferred hydrogel-linked prodrug is given by a prodrug conjugate D-L, wherein
D is a biologically active moiety comprising an amine, carboxyl, phosphate, hydroxyl or mercapto group; and
L is a non-biologically active linker containing
i) a moiety L¹ represented by formula (XV):

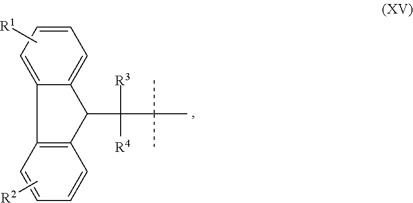

wherein the dashed line indicates the attachment of L¹ to a chemical functional group of a drug D, wherein such chemical functional group is selected from amino, carboxyl, phosphate, hydroxyl and mercapto; and wherein R¹, R², R³ and R⁴ of formula (XV) are defined as follows:
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
ii) a moiety L², which is a chemical bond or a spacer, and L² is bound to the hydrogel of the hydrogel-linked prodrug, and
   wherein L¹ is substituted with one L² moiety.

Optionally, L is further substituted.

Thus, the hydrogel is attached to any one of R¹, R², R³ or R⁴ of formula (XV), either directly (if L² is a single chemical bond) or through a spacer moiety (if L² is a spacer).

Another suitable reversible prodrug linker moiety is described in WO-A 2002/089789.

Accordingly, a preferred hydrogel-linked prodrug is shown in formula (XVI):

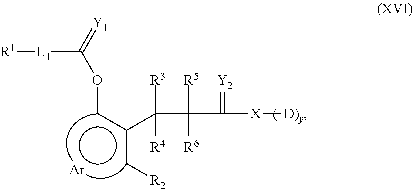

wherein D, X, y, Ar, L₁, Y₁, Y₂, R¹, R², R³, R⁴, R⁵, R⁶ of formula (XVI) are defined as follows:
D is a biologically active moiety;
L₁ is a bifunctional linking group;
Y₁ and Y₂ are independently O, S or NR⁷;
R¹ is the hydrogel;
R²⁻⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula XI forms a multisubstituted aromatic hydrocarbon or a multi-substituted heterocyclic group;
Z is either a chemical bond or a moiety that is actively transported into a target cell, a hydrophobic moiety, or a combination thereof,
y is 0 or 1;
X is a chemical bond or a moiety that is actively transported into a target cell, a hydrophobic moiety, or a combination thereof, and

Another suitable reversible prodrug linker moiety is described in WO-A 2001/47562. Accordingly, a preferred hydrogel-linked prodrug is given by formula (XVII):

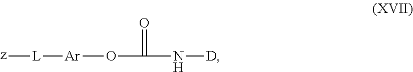

wherein D, L, z and Ar of formula (XVII) have the following meaning:
D is an amine-comprising biologically active moiety comprising NH;
L is a covalent linkage, preferably a hydrolytically stable linkage;
Ar is an aromatic group; and
z is the hydrogel.

Yet another suitable reversible prodrug linker moiety is described in U.S. Pat. No. 7,393,953 B2. Accordingly, a preferred hydrogel-linked prodrug is given by formula (XVIII):

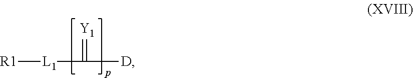

wherein R¹, L₁, Y₁, p and D of formula (XVIII) have the following meaning:
D is a heteroaromatic amine-comprising biologically active moiety connected through a heteroaromatic amine group of D to the rest of the sub-structure of formula (XVIII);
Y¹ is O, S, or NR₂;
p is 0 or 1;
L₁ is a bifunctional linker, such as, for example, —NH(CH₂CH₂O)ₘ(CH₂)ₘNR₃—, —NH(CH₂CH₂O)ₘC(O)—, —NH(CR₄R₅)ₘOC(O)—, —C(O)(CR₄R₅)ₘNHC(O)(CR₈R₇)_{q}NR₃, —C(O)O(CH₂)ₘO—, —C(O)(CR₄R₅)ₘNR₃—, —C(O)NH(CH₂CH₂O)ₘ(CH₂)ₘNR₃—, —C(O)O—(CH₂CH₂O)ₘNR₃—, —C(O)NH(CR₄R₅)ₘO—, —C(O)O(CR₄R₅)ₘO, —C(O)NH(CH₂CH₂O)ₘ—,

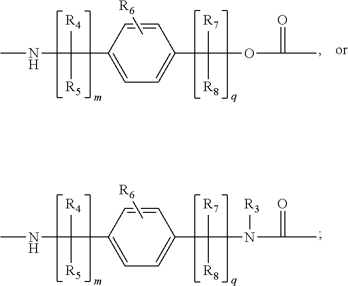

R₂, R₃, R₄, R₅, R₇ and R₈ are independently selected from the group consisting of hydrogen, C₁₋₄ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
R₆ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, NO₂, haloalkyl and halogen; and
m and q are selected independently from each other and each is a positive integer.

Another preferred hydrogel-linked prodrug is given by formula (XIX):

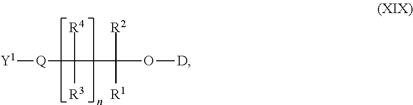

wherein D, R¹, R², R³, R⁴, Y¹ and n of formula (XIX) have the following meaning:
D is a carboxyl-comprising biologically active moiety,
R¹ is selected from the group of unsubstituted alkyl; substituted alkyl; unsubstituted phenyl; substituted phenyl; unsubstituted naphthyl; substituted naphthyl; unsubstituted indenyl; substituted indenyl; unsubstituted indanyl; substituted indanyl; unsubstituted tetralinyl; substituted tetralinyl; unsubstituted C₃₋₁₀ cycloalkyl; substituted C₃₋₁₀ cycloalkyl; unsubstituted 4- to 7-membered heterocyclyl; substituted 4- to 7-membered heterocyclyl; unsubstituted 9- to 11-membered heterobicyclyl; and substituted 9- to 11-membered heterobicyclyl;
R² is selected from H, unsubstituted alkyl, and substituted alkyl;
R³ and R⁴ are independently selected from the group consisting of H, unsubstituted alkyl, and substituted alkyl;
Q is a spacer moiety;
n is 0 or 1,
optionally, R¹ and R³ are joined together with the atoms to which they are attached to form a ring A,
A is selected from the group consisting of C₃₋₁₀ cycloalkyl; 4- to 7-membered aliphatic heterocyclyl; and 9- to 11-membered aliphatic heterobicyclyl, wherein A is unsubstituted or substituted;
Y¹ is the hydrogel.

Preferably, R¹ of formula (XIX) is C₁₋₆ alkyl or substituted C₁₋₆ alkyl, more preferably C₁₋₄ alkyl or substituted C₁₋₄ alkyl.

More preferably, R¹ of formula (XIX) is selected from methyl, ethyl, n-propyl, isopropyl, n-butyl, isobutyl, sec-butyl, t-butyl, and benzyl.

Preferably, R² of formula (XIX) is H.

Preferably, R³ of formula (XIX) is H, C₁₋₆ alkyl or substituted C₁₋₆ alkyl, more preferably C₁₋₄ alkyl or substituted C₁₋₄ alkyl. More preferably, R³ is selected from methyl, ethyl, n-propyl, isopropyl, n-butyl, isobutyl, sec-butyl, t-butyl, and benzyl.

More preferably, R³ of formula (XIX) is H.

Preferably, R⁴ of formula (XIX) is s H, C₁₋₆ alkyl or substituted C₁₋₆ alkyl, more preferably C₁₋₄ alkyl or substituted C₁₋₄ alkyl. More preferably, R⁴ is selected from methyl, ethyl, n-propyl, isopropyl, n-butyl, isobutyl, sec-butyl, t-butyl, and benzyl.

More preferably, R⁴ of formula (XIX) is H.

In another preferred embodiment, R¹ and R³ of formula (XIX) are joined together with the atoms to which they are attached to form a ring A, wherein A is selected from the group consisting of cyclopropane, cyclobutane, cyclopentane, cyclohexane, and cycloheptane.

Another preferred hydrogel-linked prodrug is given by formula (XX):

Y₁—W—O-D (XX),
wherein D, Y₁ and W of formula (XX) have the following meaning:
D is a carboxyl-comprising biologically active moiety comprising O of formula (XX),
W is selected from linear C₁₋₁₅ alkyl; and
Y¹ is the hydrogel of the hydrogel-linked prodrug.

The hydrogel-linked prodrug comprises biologically active moieties which are coupled to the hydrogel through reversible prodrug linkers and which are released intraocularly from the hydrogel-linked prodrug as drug molecules.

A list of druggable targets and preferred drugs is provided by Scheinman et al. (in: Drug Product Development for the Back of the Eye, 2011, Volume 2, 495-563), which is hereby included in its entirety.

A hydrogel-linked prodrug may comprise one or more different biologically active moieties which may be of the same or different drug classes.

Preferred biologically active moieties or drugs are selected from the group comprising: anesthetics and analgesics, antiallergenics, antihistamines, anti-inflammatory agents, anti-cancer agents, antibiotics, antiinfectives, antibacterials, anti-fungal agents, anti-viral agents, cell transport/mobility impending agents, antiglaucoma drugs, antihypertensives, decongestants, immunological response modifiers, immunosuppresive agents, peptides and proteins, steroidal compounds (steroids), low solubility steroids, carbonic anhydrize inhibitors, diagnostic agents, antiapoptosis agents, gene therapy agents, sequestering agents, reductants, antipermeability agents, antisense compounds, antiproliferative agents, antibodies and antibody conjugates, bloodflow enhancers, antiparasitic agents, non-steroidal anti inflammatory agents, nutrients and vitamins, enzyme inhibitors, antioxidants, anticataract drugs, aldose reductase inhibitors, cytoprotectants, cytokines, cytokine inhibitors, and cytokine protectants, UV blockers, mast cell stabilizers, and anti neovascular agents such as antiangiogenic agents like matrix metalloprotease inhibitors and Vascular endothelial growth factor (VEGF) modulators, neuroprotectants, miotics and anti-cholinesterase, mydriatics, artificial tear/dry eye therapies, anti-TNFα, IL-1 receptor antagonists, protein kinase C-β inhibitors, somatostatin analogs and sympathomimetics.

Non-limiting examples of preferred classes of drugs are selected from the classes of drugs comprising: antihistamines, beta-adrenoceptor antagonists, angiotensin II receptor antagonists, miotics, sympathomimetics carbonic anhydrase inhibitors, prostaglandins, antineoplastic agents, anti-microbial compounds, anti-fungal agents, anti-viral compounds, aldose reductase inhibitors, anti-inflammatory compounds, anti-allergy compounds, non-steroidal compounds, local anesthetics, peptides and proteins.

Preferred antihistamines are selected from the group comprising loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chiorcyclizine, thonzylamine, and derivatives thereof.

Preferred beta-adrenoceptor antagonists include, but are not limited to, atenalol, carteolol, cetamolol, betaxolol, levobunolol, metipranolol, timolol, acebutolol, labetalol, metoprolol, propranolol or derivatives thereof.

Preferred angiotensin II receptor antagonists include, but are not limited to, candesartan cilexetil.

Preferred miotics are selected from the group comprising for example physostigmine, pilocarpine, eserine salicylate, carbachol, di-isopropyl fluorophosphate, phospholine iodine, and demecarium bromide.

Preferred sympathomimetics include, but are not limited to, adrenaline and dipivefrine.

Preferred carbonic anhydrase inhibitors include, but are not limited to, acetazolamide, dorzolamide.

Preferred prostaglandins include, but are not limited to, bimatoprost, lantanoprost and travoprost and related compounds.

Preferred antineoplastic agents are selected from the group comprising for example adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, mitomycin C, and flutamide, and derivatives thereof.

Preferred anti-microbial compounds are selected from the group comprising for example cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefox-polyitin, cefonicid, ceforanide, ceftriaxone, cefadroxil, cephradine, cefuroxime, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, fusidic acid, gentamicin, lincomycin, tobramycin, vancomycin, polymyxin B sulfate, colistimethate, colistin, azithromycin, augmentin, sulfamethoxazole, trimethoprim, and derivatives thereof.

Preferred anti-fungal agents are, for example, selected from the compounds classes comprising polyenes, echinocandins, allylamines, imidazole, triazole, and thiazole.

Preferred anti-viral compounds include, but are not limited to, interferon alpha, interferon beta, interferon gamma, zidovudine, amantadine hydrochloride, ribavirin, acyclovir, cidofovir, idoxuridine, fomivirsen, foscarnet, valciclovir, dideoxycytidine, phosphonoformic acid, ganciclovir, and derivatives thereof.

Preferred antibiotics are selected from the group comprising ganciclovir, foscarnet, cidofovir, and fomivirsen, acyclovir, valacyclovir, vancomycin, gentamycin, clindamycin, chloramphenicol, fusidic acid.

Preferred aldose reductase inhibitors are selected from the group comprising tolrestat, epalrestat, ranirestat and fidarestat.

Anti-inflammatory compounds, e.g., steroidal compounds, are preferably selected from the group comprising cortisone, prednisolone, flurometholone, dexamethasone, medrysone, loteprednol, fluazacort, hydrocortisone, prednisone, betamethasone, clobetasone, prednisone, methylprednisolone, riamcinolone hexacatonide, paramethasone acetate, diflorasone, fluocinonide, fluocinolone, triamcinolone, derivatives thereof, and mixtures thereof. Most preferred are cortisone, prednisolone, dexamethasone, prednisone, betamethasone, methylprednisolone, fluocinonide, fluocinolone, triamcinolone, derivatives thereof, and mixtures thereof.

Preferred anti-allergy compounds include, but are not limited to, antazoline, methapyriline, chlorpheniramine, pyrilamine and prophenpyridamine.

Preferred non-steroidal compounds include, but are not limited to, antazoline, bromofenac, diclofenac, indomethacin, lodoxamide, saprofen, sodium cromoglycate.

Preferred local anesthetics include, but are not limited to amethocaine, lidocaine, lignocaine, oxbuprocaine, proxymetacaine.

Preferred peptides and proteins are selected from the group comprising cyclosporin, insulin, growth hormones, insulin related growth factor, heat shock proteins and related compounds, urogastrone and growth factors such as epidermal growth factor Another class of preferred compounds are those that modulate the CXCR4 receptor and/or SDF-I.

Also preferred drugs are antibodies, including, but are not limited to, infliximab, daclizumab, efalizumab, AIN 457, rituximab, etanecept, adalimumab and fragments thereof.

Further preferred drugs are modulators of VEGF activity, including, but not limited to, pegatinib sodium, ranibizumab, aflibercept, bevacizumab and bevasiranib sodium. Most preferred are pegatinib, ranibizumab, aflibercept, bevacizumab and bevasiranib.

Another preferred class of drugs are mydriatics, which for example include atropine sulfate, cyclopentolate, homatropine, scopolamine, tropicamide, eucatropine, and hydroxyamphetamine.

Also preferred drug are immunosuppresive agents including, but are not limited to, cyclosporine, azathioprine, tacrolimus, sirolimus, and derivatives thereof. Most preferred are sirolimus, cyclosporine, and azathioprine.

Also preferred are drugs having cycloplegic or collagenase inhibitor activity.

Another preferred class of drugs may also be photosensitizer, such as verteporfin or PPARa inhibitors, including, but are not limited to, choline fenofibrate.

Another preferred group of drugs are antioxidant agents which, for example, are selected from the group comprising ascorbate, alphatocopherol, mannitol, reduced glutathione, various carotenoids, cysteine, uric acid, taurine, tyrosine, superoxide dismutase, lutein, zeaxanthin, cryotpxanthin, astazanthin, Iycopene, N-acetyl-cysteine, carnosine, gamma-glutamylcysteine, quercitin, lactoferrin, dihydrolipoic acid, citrate, Ginkgo biloba extract, tea catechins, bilberry extract, vitamins E or esters of vitamin E, retinyl palmitate, and derivatives thereof.

Other preferred classes of drugs are integrin antagonists, selectin antagonists, adhesion molecule antagonists (such as for example Intercellular Adhesion Molecule (ICAM)-I, ICAM-2, ICAM-3, Platelet Endothelial Adhesion Molecule (PCAM), Vascular Cell Adhesion Molecule (VCAM)), or leukocyte adhesion-inducing cytokines or growth factor antagonists (such as for example growth hormone receptor antagonist, Tumor Necrosis Factor-a (TNF-a), Interleukin-1β (IL-1β), Monocyte Chemotatic Protein-1 (MCP-1) and a Vascular Endothelial Growth Factor (VEGF)).

Also preferred drugs are sub-immunoglobulin antigen-binding molecules, such as Fv immunoglobulin fragments, minibodies, and the like.

Preferred drugs are also includes PKC-inhibitors, such as, for example, ruboxistautin mesilate and AEB071.

Another preferred class of drugs are vitreolytic agents such as, for example, hyaluronidase, vitreosolve, plasmin, dispase and microlysin.

Further preferred drugs are neuroprotectants, such as, for example, nimodipine and related compounds, ciliary neurotrophic factor and related compounds, and idebenone.

Most preferred are neuroprotectants selected from the group comprising CNTF, bFGF, BDNF, GDNF, LEDGF, RdCVF, PEDF.

Additional preferred drugs are desonide, fluocinolone, fluorometholone, anecortave acetate, momethasone, fluoroquinolones, rimexolone, cephalosporin, anthracycline, aminoglycosides, sulfonamides, TNF inhibitors, anti-PDGF, mycophenolate mofetil, lenalidomide, NOS inhibitors, COX-2 inhibitors, cyclosporine A, SiRNA-027, combrestatin, combrestatin-4-phosphate, MXAA, AS1404, 2-methoxyestradiol, pegaptanib sodium, ZD6126, ZD6474, angiostatin, endostatin, anti TGF-α/β, anti IFN-α/β/γ, anti TNF-α, vasculostatin, vasostatin, angioarrestin and derivatives.

Another preferred class of drugs are plasma kallikrein inhibitors.

Preferred anti TNF-α drugs are selected from the group comprising infliximab, dalimumab, certolizumab pegol, etanercept, and golimumab.

More preferably, the hydrogel-linked prodrug comprises a biologically active moiety selected from the group comprising VEGF activity modulators, steroids, antibiotics, neuroprotectants, immunosuppresive agents, anti-TNFα, IL-1 receptor antagonists, protein kinase C-β inhibitors, and somatostatin analogs.

A preferred IL-1 receptor antagonist is anakinra.

A preferred protein kinase C-β inhibitors is ruboxistaurin.

A preferred somastatin analog is octreotide.

In another preferred embodiment, the drug may be a diagnostic agent, such as a contrast agent, known in the art.

The pharmaceutical composition comprising hydrogel-linked prodrug may be used in the prevention, diagnosis and/or treatment of multiple ocular conditions.

In one embodiment, the ocular condition affects or involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Accordingly, a preferred anterior ocular condition is selected from the group comprising aphakia, pseudophakia, astigmatism, blepharospasm, cataract, conjunctival diseases, conjunctivitis, corneal diseases, corneal ulcer, dry eye syndromes, eyelid diseases, lacrimal apparatus diseases, lacrimal duct obstruction, myopia, presbyopia, pupil disorders, refractive disorders, glaucoma and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

In another embodiment, the ocular condition is a posterior ocular condition is which primarily affects or involves a posterior ocular region or site such as choroid or sclera (in a position posterior to a plane through the posterior wall of the lens capsule), vitreous, vitreous chamber, retina, retinal pigmented epithelium, Bruch's membrane, optic nerve (i.e. the optic disc), and blood vessels and nerves which vascularize or innervate a posterior ocular region or site.

Accordingly, a preferred posterior ocular condition is selected from the group comprising acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, (such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, nonretinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e.neuroprotection).

In one embodiment the pharmaceutical composition in addition to the hydrogel-linked prodrug comprises other biologically active moieties, either in their free form or as prodrugs.

The pharmaceutical composition optionally comprises one or more excipients.

Excipients may be categorized as buffering agents, isotonicity modifiers, preservatives, stabilizers, anti-adsorption agents, oxidation protection agents, viscosifiers/viscosity enhancing agents, or other auxiliary agents. In some cases, these ingredients may have dual or triple functions. The pharmaceutical composition may contain one or more excipients, selected from the groups consisting of:
(i) Buffering agents: physiologically tolerated buffers to maintain pH in a desired range, such as sodium phosphate, bicarbonate, succinate, histidine, citrate and acetate, sulphate, nitrate, chloride, pyruvate. Antacids such as Mg(OH)₂ or ZnCO₃ may be also used. Buffering capacity may be adjusted to match the conditions most sensitive to pH stability;
(ii) Isotonicity modifiers: to minimize pain that can result from cell damage due to osmotic pressure differences at the injection depot. Glycerin and sodium chloride are examples. Effective concentrations can be determined by osmometry using an assumed osmolality of 285-315 mOsmol/kg for serum;
(iii) Preservatives and/or antimicrobials: multidose parenteral preparations require the addition of preservatives at a sufficient concentration to minimize risk of patients becoming infected upon injection and corresponding regulatory requirements have been established. Typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride;
(iv) Stabilizers: Stabilization is achieved by strengthening of the protein-stabilizing forces, by destabilization of the denatured state, or by direct binding of excipients to the protein. Stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives. In addition, oligomers or polymers such as cyclodextrins, dextran, dendrimers, PEG or PVP or protamine or HSA may be used;
(v) Anti-adsorption agents: Mainly ionic or non-ionic surfactants or other proteins or soluble polymers are used to coat or adsorb competitively to the inner surface of the composition's or composition's container. Suitable surfactants are e.g., alkyl sulfates, such as ammonium lauryl sulfate and sodium lauryl sulfate; alkyl ether sulfates, such as sodium laureth sulfate and sodium myreth sulfate; sulfonates such as dioctyl sodium sulfosuccinates, perfluorooctanesulfonates, perfluorobutanesulfonates, alkyl benzene sulfonates; phosphates, such as alkyl aryl ether phosphates and alkyl ether phosphates; carboxylates, such as fatty acid salts (soaps) or sodium stearate, sodium lauroyl sarcosinate, perfluorononanoate, perfluorooctanoate; octenidine dihydrochloride; quaternary ammonium cations such as cetyl trimethylammonium bromide, cetyl trimethylammonium chloride, cetylpyridinium chloride, polyethoxylated tallow amine, benzalkonium chloride, benzethonium chloride, 5-bromo-5-nitor-1,3-dioxane, dimethyldioctadecylammonium chloride, dioctadecyldimethylammonium bromide; zwitterionics, such as 3-[(3-cholamidopropyl)dimethylammonio]-1-propanesulfonate, cocamidopropyl hydroxysultaine, amino acids, imino acids, cocamidopropyl betaine, lecithin; fatty alcohols, such as cetyl alcohol, stearyl alcohol, cetostearyl alcohol, oleyl alcohol; polyoxyethylene glycol alkyl ethers, such as octaethylene glycol monododecyl ether, pentaethylene glycol monododecyl ether; polyoxypropylene glycol alkyl ethers; glucoside alkyl ethers, such as decyl glucoside, lauryl glucoside, octyl glucoside; polyoxyethylene glycol octylphenol ethers such as Triton X-100; polyoxyethylene glycol alkylphenol ethers such as nonoxynol-9; glycerol alkyl esters such as glyceryl laurate; polyoxyethylene glycol sorbitan alkyl esters such as polysorbates; sorbitan alkyl esters; cocamide MEA and cocamide DEA; dodecyl dimethylamine oxide; block copolymers of polyethylene glycol and polypropylene glycol, such as poloxamers (Pluronic F-68), PEG dodecyl ether (Brij 35), polysorbate 20 and 80; other anti-absorption agents are dextran, polyethylene glycol, PEG-polyhistidine, BSA and HSA and gelatines. Chosen concentration and type of excipient depends on the effect to be avoided but typically a monolayer of surfactant is formed at the interface just above the CMC value;
(vi) Lyo- and/or cryoprotectants: During freeze- or spray drying, excipients may counteract the destabilizing effects caused by hydrogen bond breaking and water removal. For this purpose sugars and polyols may be used but corresponding positive effects have also been observed for surfactants, amino acids, non-aqueous solvents, and other peptides. Trehalose is particularly efficient at reducing moisture-induced aggregation and also improves thermal stability potentially caused by exposure of protein hydrophobic groups to water. Mannitol and sucrose may also be used, either as sole lyo/cryoprotectant or in combination with each other where higher ratios of mannitol:sucrose are known to enhance physical stability of a lyophilized cake. Mannitol may also be combined with trehalose. Trehalose may also be combined with sorbitol or sorbitol used as the sole protectant. Starch or starch derivatives may also be used;
(vii) Oxidation protection agents: antioxidants such as ascorbic acid, ectoine, methionine, glutathione, monothioglycerol, morin, polyethylenimine (PEI), propyl gallate, vitamin E, chelating agents such as citric acid, EDTA, hexaphosphate, thioglycolic acid;
(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue. A spreading agent such as but not limited to hyaluronidase temporarily decreases the viscosity of the extracellular matrix and promotes diffusion of injected drugs;
(ix) Other auxiliary agents: such as wetting agents, viscosity modifiers, antibiotics, hyaluronidase. Acids and bases such as hydrochloric acid and sodium hydroxide are auxiliary agents necessary for pH adjustment during manufacture;

The pharmaceutical composition in either dry or liquid form may be provided as a single or multiple dose pharmaceutical composition.

In one embodiment of the present invention, the liquid or dry pharmaceutical composition is provided as a single dose, meaning that the container in which it is supplied contains one pharmaceutical dose.

Alternatively, the liquid or dry pharmaceutical composition is a multiple dose pharmaceutical composition, meaning that the container in which it is supplied contains more than one therapeutic dose, i.e., a multiple dose composition contains at least 2 doses. Such multiple dose pharmaceutical composition can either be used for different patients in need thereof or can be used for one patient, wherein the remaining doses are stored after the application of the first dose until needed.

In another aspect of the present invention the pharmaceutical composition is in a container. Suitable containers for liquid or dry pharmaceutical compositions are, for example, syringes, vials, vials with stopper and seal, ampoules, and cartridges. In particular, the liquid or dry pharmaceutical composition is provided in a syringe. If the pharmaceutical composition is a dry pharmaceutical composition the container preferably is a dual-chamber syringe. In such embodiment, said dry pharmaceutical composition is provided in a first chamber of the dual-chamber syringe and reconstitution solution is provided in the second chamber of the dual-chamber syringe.

Prior to applying the dry pharmaceutical composition to a patient in need thereof, the dry composition is reconstituted. Reconstitution can take place in the container in which the dry composition is provided, such as in a vial, syringe, dual-chamber syringe, ampoule, and cartridge. Reconstitution is done by adding a predefined amount of reconstitution solution to the dry composition. Reconstitution solutions are sterile liquids, such as water or buffer, which may contain further additives, such as preservatives and/or antimicrobials, such as, for example, benzylalcohol and cresol. Preferably, the reconstitution solution is sterile water. When a dry pharmaceutical composition is reconstituted, it is referred to as a “reconstituted pharmaceutical composition” or “reconstituted pharmaceutical composition” or “reconstituted composition”.

An additional aspect of the present invention relates to the method of administration of a reconstituted or liquid pharmaceutical composition comprising a hydrogel-linked prodrug for use in the prevention, diagnosis and/or treatment an ocular condition of the present invention. Preferably, the pharmaceutical composition is administered via intravitreal injection.

A further aspect is a method of preparing a reconstituted pharmaceutical composition comprising a hydrogel-linked prodrug for use in the prevention, diagnosis and/or treatment of an ocular condition, the method comprising the step of
contacting the dry pharmaceutical composition with a reconstitution solution.

Another aspect is a reconstituted pharmaceutical composition comprising a hydrogel-linked prodrug for use in the treatment, diagnosis and/or prevention an ocular condition of the present invention, and optionally one or more pharmaceutically acceptable excipients.

In case of diagnosis, the biologically active moiety is preferably a moiety which comprises at least one label, e.g. a fluorescent, phosphorescent, luminescent or radioactive label.

Another aspect of the present invention is the method of manufacturing a dry pharmaceutical composition comprising a hydrogel-linked prodrug for use in the prevention, diagnosis and/or treatment of an ocular condition. In one embodiment, such dry pharmaceutical composition is made by
(i) admixing the hydrogel-linked prodrug with optionally one or more excipients,
(ii) transferring amounts equivalent to single or multiple doses into a suitable container,
(iii) drying the pharmaceutical composition in said container, and
(iv) sealing the container.

Suitable containers are vials, syringes, dual-chamber syringes, ampoules, and cartridges. Another aspect of the present invention is a kit of parts.

If the injection device is simply a hypodermic syringe then the kit may comprise the syringe, a needle and a container comprising dry pharmaceutical composition for use with the syringe and a second container comprising the reconstitution solution.

If the pharmaceutical composition is a liquid pharmaceutical composition then the kit may comprise the syringe, a needle and a container comprising the liquid pharmaceutical composition for use with the syringe.

Another aspect of the present invention is the pharmaceutical composition for use in the prevention, diagnosis and/or treatment of an ocular condition contained in a container suited for engagement with an injection device.

In a preferred embodiment, the pharmaceutical composition of the present invention is in the form of an injection, in particular a syringe.

In more preferred embodiments, the injection device is other than a simple hypodermic syringe and so the separate container with reconstituted or liquid pharmaceutical composition is adapted to engage with the injection device such that in use the liquid pharmaceutical composition in the container is in fluid connection with the outlet of the injection device. Examples of injection devices include but are not limited to hypodermic syringes and pen injector devices. Particularly preferred injection devices are the pen injectors in which case the container is a cartridge, preferably a disposable cartridge. Optionally, the kit of parts comprises a safety device for the needle which can be used to cap or cover the needle after use to prevent injury.

A preferred kit of parts comprises a needle and a container containing the pharmaceutical composition and optionally further containing a reconstitution solution, the container being adapted for use with the needle. Preferably, the container is a dual-chamber syringe.

Another aspect of the present invention is an ophthalmic device comprising at least one pharmaceutical composition of the present invention. Preferably, such ophthalmic device is a syringe with a needle, more preferably with a thin needle, such as a needle smaller than 0.6 mm inner diameter, preferably a needle smaller than 0.3 mm inner diameter, more preferably a needle small than 0.25 mm inner diameter, even more preferably a needle smaller than 0.2 mm inner diameter, and most preferably a needle small than 0.16 mm inner diameter.

The present invention also relates to a pharmaceutical composition comprising a hydrogel-linked prodrug for the preparation of a medicament for the prevention, diagnosis and/or treatment of an ocular condition.

The present invention also relates to a hydrogel-linked prodrug of the present invention for use in the prevention, diagnosis and/or treatment of an ocular condition.

The present invention also relates to a method of preventing and/or treating an ocular disease, wherein said method comprises the step of administering a therapeutically effective amount of a hydrogel-linked prodrug of the present invention to a patient in need thereof. Preferably, the pharmaceutical composition is administered by intraocular injection, more preferably by intravitreal injection into the vitreous body.

The hydrogel-linked prodrugs of the present invention can be synthesized in a number of ways using standard chemical procedures. The hydrogel carrier may be generated through chemical ligation reactions. In one alternative, the starting material is one macromolecular starting material with complementary functionalities which undergo a reaction such as a condensation or addition reaction, which is a heteromultifunctional backbone reagent, comprising a number of polymerizable functional groups.

Alternatively, the hydrogel may be formed from two or more macromolecular starting materials with complementary functionalities which undergo a reaction such as a condensation or addition reaction. One of these starting materials is a crosslinker reagent with at least two identical polymerizable functional groups and the other starting material is a homomultifunctional or heteromultifunctional backbone reagent, also comprising a number of polymerizable functional groups.

Suitable polymerizable functional groups present on the crosslinker reagent include terminal primary and secondary amino, carboxylic acid and derivatives, maleimide, thiol, hydroxyl and other alpha,beta unsaturated Michael acceptors like vinylsulfone groups. Suitable polymerizable functional groups present in the backbone reagent include but are not limited to primary and secondary amino, carboxylic acid and derivatives, maleimide, thiol, hydroxyl and other alpha,beta unsaturated Michael acceptors like vinylsulfone groups.

If the crosslinker reagent polymerizable functional groups are used substoichiometrically with respect to backbone polymerizable functional groups, the resulting biodegradable hydrogel will be a reactive biodegradable hydrogel with free reactive functional groups attached to the backbone structure, i.e. to backbone moieties.

The hydrogel-linked prodrugs may be obtained by first conjugating a reversible prodrug linker moiety which carries protecting groups to a drug moiety and the resulting biologically active moiety-reversible prodrug linker conjugate may then be deprotected and reacted with the biodegradable hydrogel's reactive functional groups or the chemical functional groups of a spacer moiety.

If the drug is a protein drug, protein-compatible protecting groups, i.e. protecting groups which can be removed under mild aqueous conditions and which do not harm or inactivate the protein, should be used. Suitable examples for such protein-compatible protecting groups are acetyls for the protection of thiol groups which can be removed using an aqueous buffer containing hydroxylamine or a suitable protecting group for the protection of amines which can be removed under slightly basic conditions. The latter protecting group may also be left in place to yield a double prodrug, i.e. a prodrug from which two promoieties are subsequently cleaved off to release the free drug.

Alternatively, one of the chemical functional groups of the reversible prodrug linker moiety is activated first and the activated reversible prodrug linker moiety is reacted with the hydrogel's reactive functional groups or the chemical functional groups of a spacer moiety. Subsequently, the reversible linker is optionally activated again and the drug coupled to the reversible prodrug linker attached to the hydrogel.

### Operative Examples

The subject matter of the present invention is elucidated in more detail below, using examples, without any intention that the subject matter of the invention should be confined to these exemplary embodiments.

Materials and Methods

Amino 4-arm PEG 5 kDa was obtained from JenKem Technology, Beijing, P. R. China. Cithrol™ DPHS was obtained from Croda International Pic, Cowick Hall, United Kingdom.

cis-1,4-cyclohexanedicaboxylic acid was obtained from TCI EUROPE N.V., Boerenveldseweg 6 —Haven 1063, 2070 Zwijndrecht, Belgium.

Isopropylmalonic acid was obtained from ABCR GmbH & Co. KG, 76187 Karlsruhe, Germany.

N-(3-maleimidopropyl)-39-amino-4,7,10,13,16,19,22,25,28,31,34,37-dodecaoxa-nonatriacontanoic acid pentafluorophenyl ester (Mal-PEG12-PFE) was obtained from Biomatrik Inc., Jiaxing, P. R. China. All other chemicals were from Sigma-ALDRICH Chemie GmbH, Taufkirchen, Germany.

N-(3-maleimidopropyl)-21-amino-4,7,10,13,16,19-hexaoxa-heneicosanoic acid NHS ester (Mal-PEG6-NHS) was obtained from Celares GmbH, Berlin, Germany.

6-(S-Tritylmercapto)hexanoic acid was purchased from Polypeptide, Strasbourg, France. All other chemicals were from Sigma-ALDRICH Chemie GmbH, Taufkirchen, Germany.

15-Tritylthio-4,7,10,13-tetraoxa-pentadecanoic acid (Trt-S-PEG4-COOH) is obtained from Iris Biotech GmbH, Marktredwitz, Germany.

Oxyma pure and Fmoc-L-Asp(OtBu)-OH were purchased from Merck Biosciences GmbH, Schwalbach/Ts, Germany.

(5-methyl-2-oxo-1,3-dioxol-4-yl)-methyl 4-nitrophenyl carbonate was purchased from Chemzon Scientific Inc., Lachine, QC, Canada.

Methods:

Fmoc Deprotection:

For Fmoc protecting-group removal, the resin was agitated with 2/2/96 (v/v/v) piperidine/DBU/DMF (two times, 10 min each) and washed with DMF (ten times).

RP-HPLC Purification:

RP-HPLC was done on a 100×20 mm or 100×40 mm C18 ReproSil-Pur 300 ODS-3 5 μm column (Dr. Maisch, Ammerbuch, Germany) connected to a Waters 600 HPLC System and Waters 2487 Absorbance detector unless otherwise stated. Linear gradients of solution A (0.1% TFA in H₂O) and solution B (0.1% TFA in acetonitrile) were used. HPLC fractions containing product were pooled and lyophilized.

Flash Chromatography

Flash chromatography purifications were performed on an Isolera One system from Biotage AB, Sweden, using Biotage KP-Sil silica cartridges and n-heptane, ethyl acetate, and methanol as eluents. Products were detected at 254 nm. For products showing no absorbance above 240 nm fractions were screened by LC/MS.

For hydrogel beads, syringes equipped with polyethylene frits were used as reaction vessels or for washing steps.

Analytical ultra-performance LC (UPLC) was performed on a Waters Acquity system equipped with a Waters BEH300 C18 column (2.1×50 mm, 1.7 μm particle size) coupled to a LTQ Orbitrap Discovery mass spectrometer from Thermo Scientific.

HPLC-Electrospray ionization mass spectrometry (HPLC-ESI-MS) was performed on a Waters Acquity UPLC with an Acquity PDA detector coupled to a Thermo LTQ Orbitrap Discovery high resolution/high accuracy mass spectrometer equipped with a Waters ACQUITY UPLC BEH300 C18 RP column (2.1×50 mm, 300 Å, 1.7 μm, flow: 0.25 mL/min; solvent A: UP—H₂O+0.04% TFA, solvent B: UP-Acetonitrile+0.05% TFA.

MS of PEG products showed a series of (CH₂CH₂O)ₙ moieties due to polydispersity of PEG starting materials. For easier interpretation only one single representative m/z signal is given in the examples.

### Example 1

### Synthesis of Backbone Reagent 1g

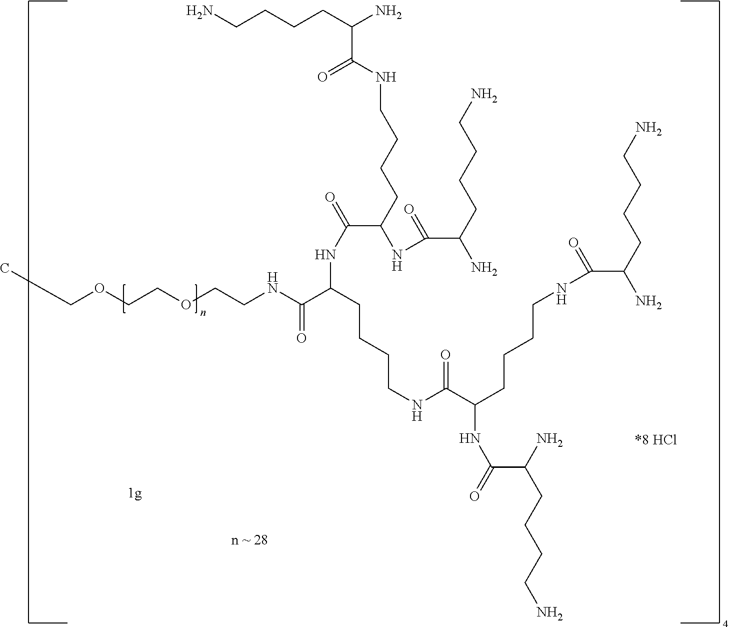

Backbone reagent 1g was synthesized from amino 4-arm PEG5000 1a according to following scheme:

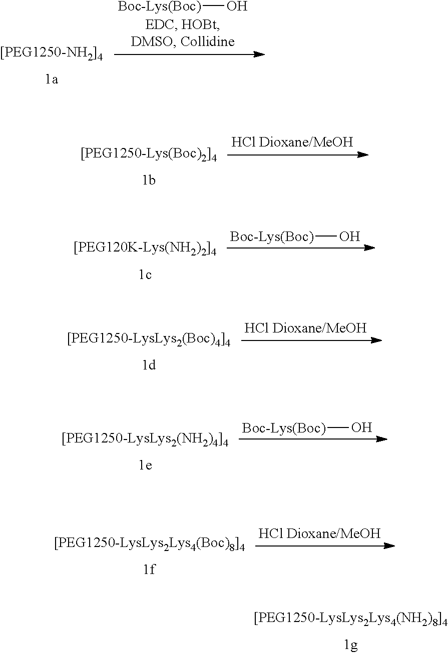

For synthesis of compound 1b, amino 4-arm PEG5000 1a (MW ca. 5200 g/mol, 5.20 g, 1.00 mmol, HCl salt) was dissolved in 20 mL of DMSO (anhydrous). Boc-Lys(Boc)-OH (2.17 g, 6.25 mmol) in 5 mL of DMSO (anhydrous), EDC HCl (1.15 g, 6.00 mmol), HOBt.H₂O (0.96 g, 6.25 mmol), and collidine (5.20 mL, 40 mmol) were added. The reaction mixture was stirred for 30 min at RT.

The reaction mixture was diluted with 1200 mL of DCM and washed with 600 mL of 0.1 N H₂SO₄ (2×), brine (1×), 0.1 M NaOH (2×), and 1/1 (v/v) brine/water (4×). Aqueous layers were reextracted with 500 mL of DCM. Organic phases were dried over Na₂SO₄, filtered and evaporated to give 6.3 g of crude product 1b as colorless oil. Compound 1b was purified by RP-HPLC.

Yield 3.85 g (59%) colorless glassy product 1b.

MS: m/z 1294.4=[M+5H]⁵⁺ (calculated=1294.6).

Compound 1c was obtained by stirring of 3.40 g of compound 1b (0.521 mmol) in 5 mL of methanol and 9 mL of 4 N HCl in dioxane at RT for 15 min. Volatiles were removed in vacuo. The product was used in the next step without further purification.

MS: m/z 1151.9=[M+5H]⁵⁺ (calculated=1152.0).

For synthesis of compound 1d, 3.26 g of compound 1c (0.54 mmol) were dissolved in 15 mL of DMSO (anhydrous). 2.99 g Boc-Lys(Boc)-OH (8.64 mmol) in 15 mL DMSO (anhydrous), 1.55 g EDC HCl (8.1 mmol), 1.24 g HOBt.H₂O (8.1 mmol), and 5.62 mL of collidine (43 mmol) were added. The reaction mixture was stirred for 30 min at RT. Reaction mixture was diluted with 800 mL DCM and washed with 400 mL of 0.1 N H₂SO₄ (2×), brine (1×), 0.1 M NaOH (2×), and 1/1 (v/v) brine/water (4×). Aqueous layers were reextracted with 800 mL of DCM. Organic phases were dried with Na₂SO₄, filtered and evaporated to give a glassy crude product.

Product was dissolved in DCM and precipitated with cooled (−18° C.) diethylether. This procedure was repeated twice and the precipitate was dried in vacuo.

Yield: 4.01 g (89%) colorless glassy product 1d, which was used in the next step without further purification.

MS: m/z 1405.4=[M+6H]⁶⁺ (calculated=1405.4).

Compound 1e was obtained by stirring a solution of compound 1d (3.96 g, 0.47 mmol) in 7 mL of methanol and 20 mL of 4 N HCl in dioxane at RT for 15 min. Volatiles were removed in vacuo. The product was used in the next step without further purification.

MS: m/z 969.6=[M+7H]⁷⁺ (calculated=969.7).

For the synthesis of compound if, compound 1e (3.55 g, 0.48 mmol) was dissolved in 20 mL of DMSO (anhydrous). Boc-Lys(Boc)-OH (5.32 g, 15.4 mmol) in 18.8 mL of DMSO (anhydrous), EDC HCl (2.76 g, 14.4 mmol), HOBt.H₂O (2.20 g, 14.4 mmol), and 10.0 mL of collidine (76.8 mmol) were added. The reaction mixture was stirred for 60 min at RT.

The reaction mixture was diluted with 800 mL of DCM and washed with 400 mL of 0.1 N H₂SO₄ (2×), brine (1×), 0.1 M NaOH (2×), and 1/1 (v/v) brine/water (4×). Aqueous layers were reextracted with 800 mL of DCM. Organic phases were dried over Na₂SO₄, filtered and evaporated to give crude product if as colorless oil.

Product was dissolved in DCM and precipitated with cooled (−18° C.) diethylther. This step was repeated twice and the precipitate was dried in vacuo.

Yield: 4.72 g (82%) colourless glassy product if which was used in the next step without further purification.

MS: m/z 1505.3=[M+8H]⁹⁺ (calculated=1505.4).

Backbone reagent 1g was obtained by stirring a solution of compound if (MW ca. 12035 g/mol, 4.72 g, 0.39 mmol) in 20 mL of methanol and 40 mL of 4 N HCl in dioxane at RT for 30 min. Volatiles were removed in vacuo.

Yield: 3.91 g (100%), glassy product backbone reagent 1g.

MS: m/z 977.2=[M+9H]⁹⁺ (calculated=977.4).

Alternative Synthetic Route for 1g

For synthesis of compound 1b, to a suspension of 4-Arm-PEG5000 tetraamine (1a) (50.0 g, 10.0 mmol) in 250 mL of iPrOH (anhydrous), boc-Lys(boc)-OSu (26.6 g, 60.0 mmol) and DIEA (20.9 mL, 120 mmol) were added at 45° C. and the mixture was stirred for 30 min.

Subsequently, n-propylamine (2.48 mL, 30.0 mmol) was added. After 5 min the solution was diluted with 1000 mL of MTBE and stored overnight at −20° C. without stirring. Approximately 500 mL of the supernatant were decanted and discarded. 300 mL of cold MTBE were added and after 1 min shaking the product was collected by filtration through a glass filter and washed with 500 mL of cold MTBE. The product was dried in vacuo for 16 h.

Yield: 65.6 g (74%) 1b as a white lumpy solid

MS: m/z 937.4=[M+7H]⁷⁺ (calculated=937.6).

Compound 1c was obtained by stirring of compound 1b from the previous step (48.8 g, 7.44 mmol) in 156 mL of 2-propanol at 40° C. A mixture of 196 mL of 2-propanol and 78.3 mL of acetylchloride was added under stirring within 1-2 min. The solution was stirred at 40° C. for 30 min and cooled to −30° C. overnight without stirring. 100 mL of cold MTBE were added, the suspension was shaken for 1 min and cooled for 1 h at −30° C. The product was collected by filtration through a glass filter and washed with 200 mL of cold MTBE. The product was dried in vacuo for 16 h.

Yield: 38.9 g (86%) 1c as a white powder

MS: m/z 960.1=[M+6H]⁶⁺ (calculated=960.2).

For synthesis of compound 1d, boc-Lys(boc)-OSu (16.7 g, 37.7 mmol) and DIPEA (13.1 mL, 75.4 mmol) were added to a suspension of 1c from the previous step (19.0 g, 3.14 mmol) in 80 ml 2-propanol at 45° C. and the mixture was stirred for 30 min at 45° C. Subsequently, n-propylamine (1.56 mL, 18.9 mmol) was added. After 5 min the solution was precipitated with 600 mL of cold MTBE and centrifuged (3000 min⁻¹, 1 min) The precipitate was dried in vacuo for 1 h and dissolved in 400 mL THF. 200 mL of diethyl ether were added and the product was cooled to −30° C. for 16 h without stirring. The suspension was filtered through a glass filter and washed with 300 mL cold MTBE. The product was dried in vacuo for 16 h.

Yield: 21.0 g (80%) 1d as a white solid

MS: m/z 1405.4=[M+6H]⁶⁺ (calculated=1405.4).

Compound 1e was obtained by dissolving compound 1d from the previous step (15.6 g, 1.86 mmol) in 3 N HCl in methanol (81 mL, 243 mmol) and stirring for 90 min at 40° C. 200 mL of MeOH and 700 mL of iPrOH were added and the mixture was stored for 2 h at −30° C. For completeness of crystallization, 100 mL of MTBE were added and the suspension was stored at −30° C. overnight. 250 mL of cold MTBE were added, the suspension was shaken for 1 min and filtered through a glass filter and washed with 100 mL of cold MTBE. The product was dried in vacuo.

Yield: 13.2 g (96%) 1e as a white powder

MS: m/z 679.1=[M+10H]¹⁰⁺ (calculated=679.1).

For the synthesis of compound if, boc-Lys(boc)-OSu (11.9 g, 26.8 mmol) and DIPEA (9.34 mL, 53.6 mmol) were added to a suspension of 1e from the previous step, (8.22 g, 1.12 mmol) in 165 ml 2-propanol at 45° C. and the mixture was stirred for 30 min. Subsequently, n-propylamine (1.47 mL, 17.9 mmol) was added. After 5 min the solution was cooled to −18° C. for 2 h, then 165 mL of cold MTBE were added, the suspension was shaken for 1 min and filtered through a glass filter. Subsequently, the filter cake was washed with 4×200 mL of cold MTBE/iPrOH 4:1 and 1×200 mL of cold MTBE. The product was dried in vacuo for 16 h.

Yield: 12.8 g, MW (90%) if as a pale yellow lumpy solid

MS: m/z 1505.3=[M+8H]⁸⁺ (calculated=1505.4).

Backbone reagent 1g was obtained by dissolving 4ArmPEG5 kDa(-LysLys₂Lys₄(boc)₈)₄ (if) (15.5 g, 1.29 mmol) in 30 mL of MeOH and cooling to 0° C. 4 N HCl in dioxane (120 mL, 480 mmol, cooled to 0° C.) was added within 3 min and the ice bath was removed. After 20 min, 3 N HCl in methanol (200 mL, 600 mmol, cooled to 0° C.) was added within 15 min and the solution was stirred for 10 min at room temperature. The product solution was precipitated with 480 mL of cold MTBE and centrifuged at 3000 rpm for 1 min. The precipitate was dried in vacuo for 1 h and redissolved in 90 mL of MeOH, precipitated with 240 mL of cold MTBE and the suspension was centrifuged at 3000 rpm for 1 min. The product 1g was dried in vacuo Yield: 11.5 g (89%) as pale yellow flakes.

MS: m/z 1104.9=[M+8H]⁸⁺ (calculated=1104.9).

### Example 2

Synthesis of Crosslinker Reagent 2d

Crosslinker reagent 2d was prepared from adipic acid mono benzyl ester (English, Arthur R. et al., Journal of Medicinal Chemistry, 1990, 33(1), 344-347) and PEG2000 according to the following scheme:

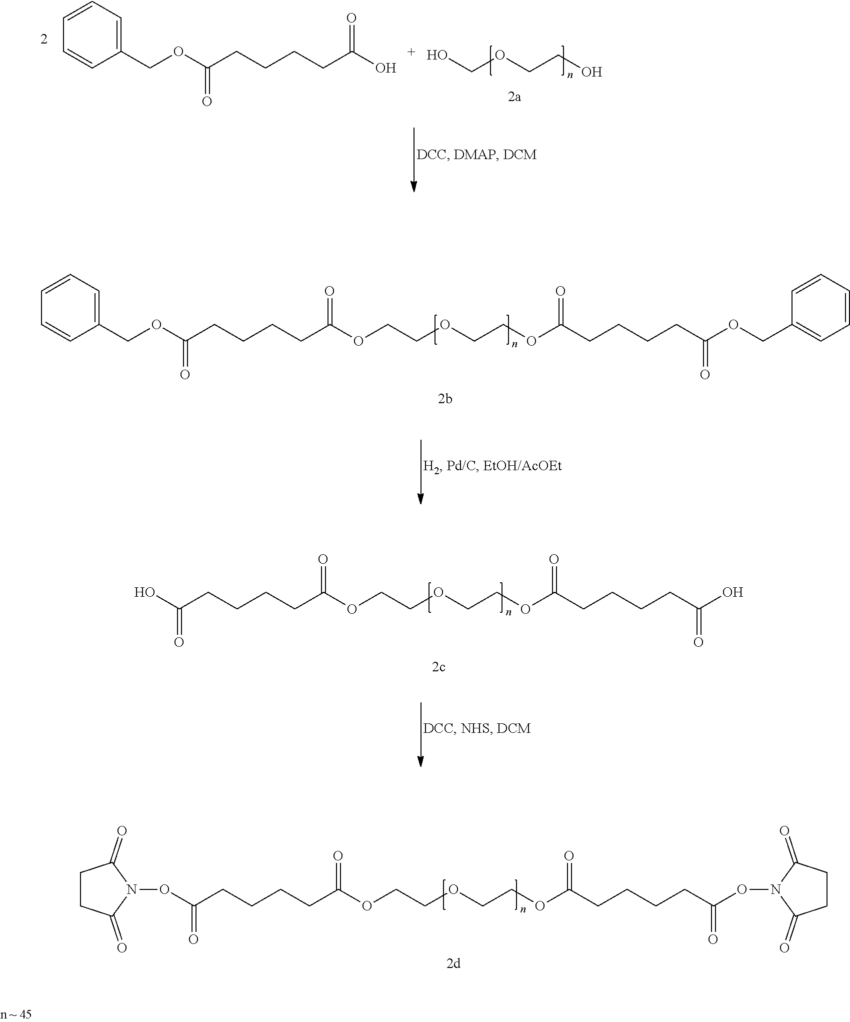

A solution of PEG 2000 (2a) (11.0 g, 5.5 mmol) and benzyl adipate half-ester (4.8 g, 20.6 mmol) in DCM (90.0 mL) was cooled to 0° C. Dicyclohexylcarbodiimide (4.47 g, 21.7 mmol) was added followed by a catalytic amount of DMAP (5 mg) and the solution was stirred and allowed to reach room temperature overnight (12 h). The flask was stored at +4° C. for 5 h. The solid was filtered and the solvent completely removed by distillation in vacuo. The residue was dissolved in 1000 mL 1/1 (v/v) diethyl ether/ethyl acetate and stored at RT for 2 hours while a small amount of a flaky solid was formed. The solid was removed by filtration through a pad of Celite®. The solution was stored in a tightly closed flask at −30° C. in the freezer for 12 h until crystallisation was complete. The crystalline product was filtered through a glass frit and washed with cooled diethyl ether (−30° C.). The filter cake was dried in vacuo.

Yield: 11.6 g (86%) 2b as a colorless solid. The product was used without further purification in the next step.

MS: m/z 813.1=[M+3H]³⁺ (calculated=813.3) In a 500 mL glass autoclave PEG2000-bis-adipic acid-bis-benzyl ester 2b (13.3 g, 5.5 mmol) was dissolved in ethyl acetate (180 mL) and 10% Palladium on charcoal (0.4 g) was added. The solution was hydrogenated at 6 bar, 40° C. until consumption of hydrogen had ceased (5-12 h). Catalyst was removed by filtration through a pad of Celite® and the solvent was evaporated in vacuo.

Yield: 12.3 g (quantitative) 2c as yellowish oil. The product was used without further purification in the next step.

MS: m/z 753.1=[M+3H]³⁺ (calculated=753.2) A solution of PEG2000-bis-adipic acid half ester 2c (9.43 g, 4.18 mmol), N-hydroxysuccinimide (1.92 g, 16.7 mmol) and dicyclohexylcarbodiimide (3.44 g, 16.7 mmol) in 75 mL of DCM (anhydrous) was stirred over night at room temperature. The reaction mixture was cooled to 0° C. and precipitate was filtered off. DCM was evaporated and the residue was recrystallized from THF.

Yield: 8.73 g (85%) crosslinker reagent 2d as colorless solid.

MS: m/z 817.8=[M+3H]³⁺ (calculated=817.9 g/mol).

Synthesis of 2e

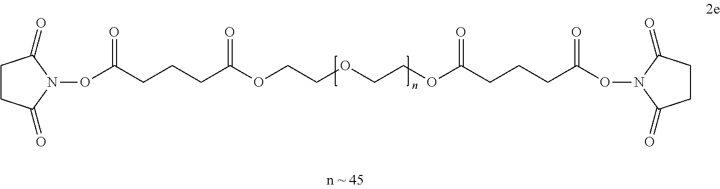

2e was synthesized as described for 2d except for the use of glutaric acid instead of adipic acid

MS: m/z 764.4=[M+3H]3+ (calculated=764.5).

### Example 3

Preparation of hydrogel beads 3 containing free amino groups A solution of 1200 mg 1g and 3840 mg 2e in 28.6 mL DMSO was added to a solution of 425 mg Arlacel P135 (Croda International Plc) in 100 mL heptane. The mixture was stirred at 650 rpm with a propeller stirrer for 10 min at 25° C. to form a suspension in a 250 ml reactor equipped with baffles. 4.3 mL TMEDA was added to effect polymerization. After 2 h, the stirrer speed was reduced to 400 rpm and the mixture was stirred for additional 16 h. 6.6 mL of acetic acid were added and then after 10 min 50 mL of water and 50 mL of saturated aqueous sodium chloride solution were added. After 5 min, the stirrer was stopped and the aqueous phase was drained.

For bead size fractionation, the water-hydrogel suspension was wet-sieved on 75, 50, 40, 32 and 20 μm mesh steel sieves. Bead fractions that were retained on the 32, 40, and 50 μm sieves were pooled and washed 3 times with water, 10 times with ethanol and dried for 16 h at 0.1 mbar to give 3 as a white powder.

Amino group content of hydrogel was determined by coupling of a fmoc-amino acid to the free amino groups of the hydrogel and subsequent fmoc-determination as described by Gude, M., J. Ryf, et al. (2002) Letters in Peptide Science 9(4): 203-206.

The amino group content of 3 was determined to be between 0.11 and 0.16 mmol/g.

### Example 4

Preparation of Maleimide Functionalized Hydrogel Suspension 4 and Determination of Maleimide Substitution

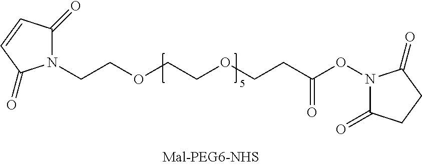

Hydrogel 3 was pre-washed with 99/1 (v/v) DMSO/DIPEA, washed with DMSO and incubated for 45 min with a solution of Mal-PEG6-NHS (2.0 eq relative to theoretical amount of amino groups on hydrogel) in DMSO. Hydrogel were washed five times with DMSO and five times with pH 3.0 succinate (20 mM, 1 mM EDTA, 0.01% Tween-20). The sample was washed three times with pH 6.0 sodium phosphate (50 mM, 50 mM ethanolamine, 0.01% Tween-20) and incubated in the same buffer for 1 h at RT. Then hydrogel was washed five times with pH 3.0 sodium succinate (20 mM, 1 mM EDTA, 0.01% Tween-20) and kept in that buffer to yield maleimide functionalized hydrogel 4 in suspension.

For determination of maleimide content, an aliquot of hydrogel 4 was washed three times with water and ethanol each. The aliquot was dried under reduced pressure and the weight of hydrogel in the aliquot was determined. Another aliquot of hydrogel 4 was reacted with excess mercaptoethanol (in 50 mM sodium phosphate buffer, 30 min at RT), and mercaptoethanol consumption was detected by Ellman test (Ellman, G. L. et al., Biochem. Pharmacol., 1961, 7, 88-95). A maleimide content of 0.10-0.15 mmol/g dried hydrogel was calculated.

### Example 5

Preparation of Betamethasone Linker Reagent 5 Betamethasone linker reagent 5 is synthesized according to the following scheme:

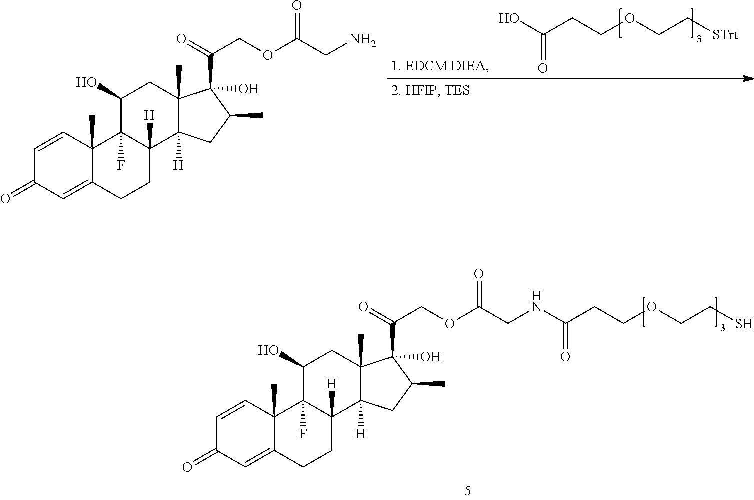

21-Glycyl-betamethasone is prepared according to the literature (Benedini, Francesca; Biondi, Stefano; Ongini, Ennio, PCT Int. Appl. (2008), WO 2008095806 A1 20080814). To a solution of 21-glycyl-betamethasone hydrochloride (MW 486 g/mol, 600 mg, 1.2 mmol) in methylene chloride (dry, molecular sieve, 40 ml), Trt-S-PEG4-COOH (MW 480.6 g/mol, 960 mg, 2.0 mmol) and DIEA (129.2 g/mol, d 0.742 mg/mL, 0.7 ml, 4 mmol) are added. The reaction is stirred at room temperature for 24 h. The solution is treated with a 5% solution of H3PO4 (50 ml). The organic layer is dried over sodium sulfate and concentrated under reduced pressure. The residue is dissolved in 2 mL dichloro methane and 8 mL HFIP. 0.4 mL TES are added and the reaction is stirred at room temperature for 1 h. Volatiles are removed under reduced pressure and 5 is purified by RP-HPLC.

### Example 6

Synthesis of Betamethasone Linker Hydrogel 6

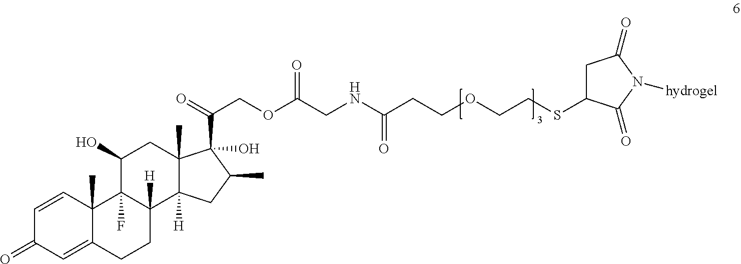

A suspension of maleimide functionalized hydrogel 4 in pH 3.0 succinate buffer (20 mM, 1 mM EDTA, 0.01% Tween-20)/acetonitrile 1/2 (v/v), (corresponding to 250 mg dried hydrogel, maleimide loading of 0.1 mmol/g dried hydrogel) is filled into a syringe equipped with a filter frit. The hydrogel is washed ten times with 2/1 (v/v) acetonitrile/water containing 0.1% TFA (v/v). A solution of betamethasone linker reagent 6 (MW 669.8 g/mol, 18.5 mg, 27.5 μmol) in 2/1 (v/v) acetonitrile/water containing 0.1% TFA (3.7 mL) is drawn up and shaken for 2 min at RT to obtain an equilibrated suspension. 334 μL phosphate buffer (pH 7.4, 0.5 M) is added and the syringe is agitated at RT. Consumption of thiol is monitored by Ellman test. The hydrogel is washed 10 times with 1/1 (v/v) acetonitrile/water containing 0.1% TFA (v/v). Mercaptoethanol (47 μL) is dissolved in 1/1 (v/v) acetonitrile/water plus 0.1% TFA (3 mL) and phosphate buffer (0.5 mL, pH 7.4, 0.5 M). The solution is drawn into the syringe and the syringe is agitated for 30 min at RT. Hydrogel is washed ten times with 1/1 (v/v) acetonitrile/water plus 0.1% TFA and ten times with sterile succinate buffer (10 mM, 46 g/L mannitol, 0.05% Tween-20, adjusted to pH 5.0 with 5 M NaOH). Volume is adjusted to 5 mL to yield 50 mg/mL betamethasone linker hydrogel 6 as suspension in succinate buffer.

Betamethasone content is determined by thiol consumption during reaction (Ellman test).

### Example 7

Release Kinetics In Vitro

An aliquot of betamethasone linker hydrogel 6 is transferred in a syringe equipped with a filter frit and washed 5 times with pH 7.4 phosphate buffer (60 mM, 3 mM EDTA, 0.01% Tween-20). The hydrogel is suspended in the same buffer and incubated at 37° C. At defined time points (after 1-7 days incubation time each) the supernatant is exchanged and liberated betametasone is quantified by RP-HPLC at 215 nm. UV-signals correlating to liberated betamethasone are integrated and plotted against incubation time.

Curve-fitting software is applied to estimate the corresponding halftime of release.

### Example 8

Synthesis of Acetylated Hydrogel 8

Hydrogel 3 (0.5 g, 62 μmol amino groups) was given in a 20 mL syringe equipped with a filter frit, NMP was added (15 mL) and the syringes were placed on an orbital shaker for 5 min. The supernatant was released, 1 mL acylation mixture (417 mM acetic anhydride, 833 mM N,N-diisopropylethylamine in NMP) was drawn into the syringe, and placed for 30 min on an orbital shaker. The supernatant was released and the acylation reaction was repeated as described above. Acetylated hydrogel 8 was washed 10 times with NMP, 10 times with 0.1% acetic acid and 10 times with NMP.

### Example 9

Preparation of Acetylated Hydrogel Suspension 9 for Intravitreal Injection

Acetylated hydrogel 8 (0.5 g) in a 20 mL syringe equipped with a filter frit was filled-up to 10 mL suspension with NMP and subjected to gamma sterilization (34 kGy). Under sterile conditions, NMP was removed by washing 15 times with sterile histidine buffer (10 mM histidine, 10% α, α-trehalose dihydrate, 0.01% polysorbate 20, adjusted to pH 5.5 with 5 M HCl). After the last wash, injection buffer was added to prepare 6 mL hydrogel suspension 6 containing approx. 80 mg acetylated hydrogel/mL.

### Example 10

Local Tolerance Study of Hydrogel after Intravitreal Injection in Rabbits

50 μL of hydrogel suspension 9 was injected intravitreously in the right eye of 12 anesthesized male New Zealand White rabbits via 30 G needle. 50 μl control item histidine buffer was injected intravitreously in the left eye. Three animals each were euthanized 1, 3, 7 and 14 days after dosing. Eyes were trimmed, frozen, and stained with hematoxylin and eosin (H&E). Tissues were evaluated by light microscopy.

In the right eyes, basophilic spheres consistent with hydrogel was present in the vitreous chamber towards the ventral side (2 of 12 animals) or in the central part (10 of 12 animals). There was no inflammation associated with the foreign material and no other microscopic changes were present in the eye. The histopathological evaluation of the left eyes revealed no evidence of an inflammatory response to the control item.

### Example 11

Pharmacokinetics and Retinal Distribution of Betamethasone after Intravitreal Injection of Betamethasone Linker Hydrogel in Rabbits

50 μL of hydrogel suspension 6 is injected intravitreously in the right eye of 18 anesthesized male New Zealand White rabbits via 28 G needle in both eyes. Two animals each are euthanized 1 and 8 h and 1, 3, 7, 14, 21, 28 and 42 days after dosing. Whole blood is collected via the medial ear artery or cardiac bleed under anesthesia. Vitreous and aqueous humor is collected from both eyes. Betamethasone is quantified by liquid chromatography-tandem mass spectrometry according to literature (Pereira Ados S, Oliveira L S, Mendes G D, Gabbai J J, De Nucci G. Quantification of betamethasone in human plasma by liquid chromatography-tandem mass spectrometry using atmospheric pressure photoionization in negative mode, J Chromatogr B Analyt Technol Biomed Life Sci. 2005 Dec. 15; 828(1-2):27-32.).

### Example 12

Synthesis of Backbone Reagent 12a and 12g:

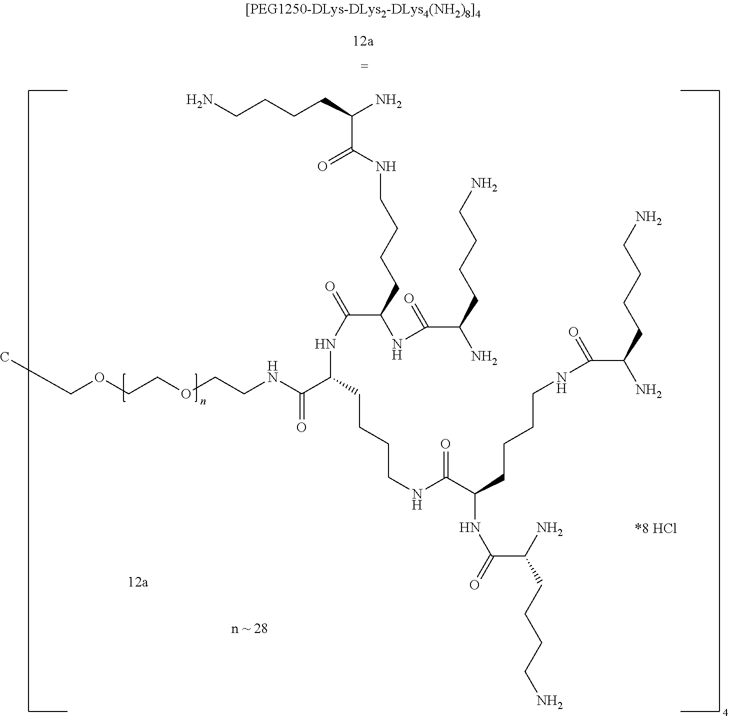

Backbone reagent 12a was synthesized as described in example 1 of WO 2011/012715 A1 except for the use of Boc-DLys(Boc)-OH instead of Boc-LLys(Boc)-OH.

MS: m/z 888.50=[M+10H+]¹⁰⁺ (calculated=888.54)

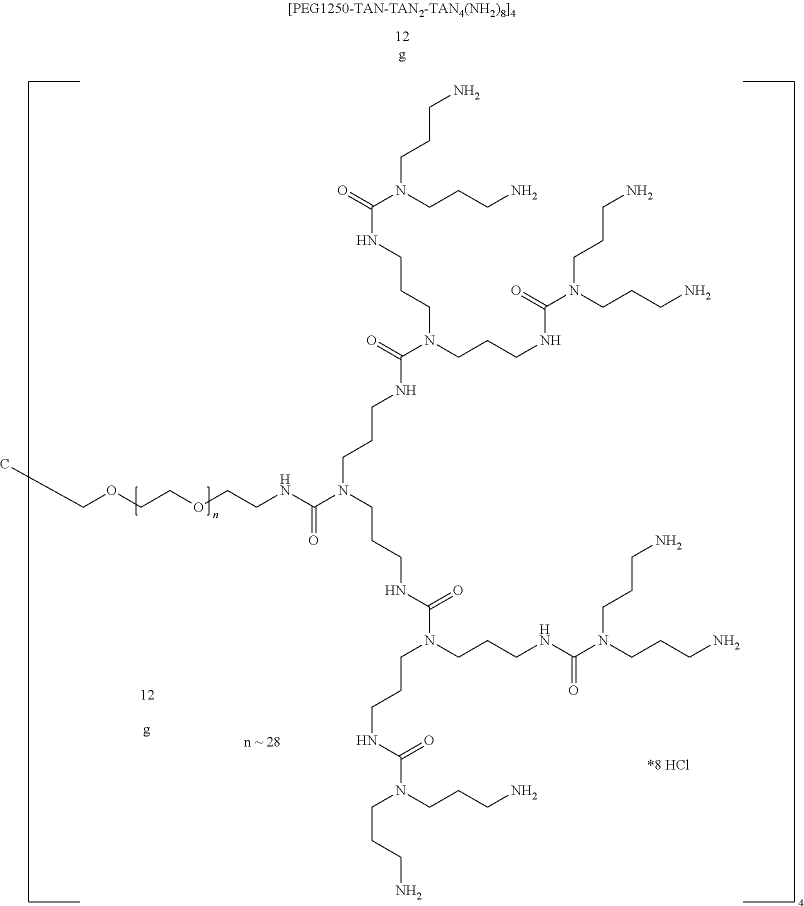

Backbone reagent 12g was synthesized from amino 4-arm PEG5000 12b according to the following scheme:

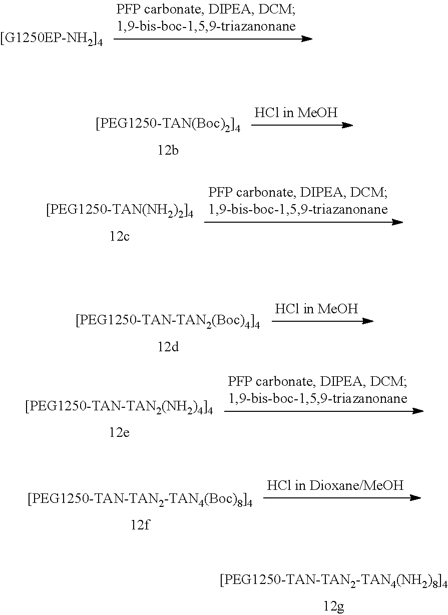

For synthesis of compound 12b, amino 4-arm PEG5000 (MW ca. 5350 g/mol, 10.7 g, 2.00 mmol, HCl salt) and bis(pentafluorophenyl)carbonate (4.73 g, 12.0 mmol) were dissolved in 43 mL of DCM (anhydrous) and DIPEA (3.10 g, 24.0 mmol, 4.18 mL) was added at room temperature. After 10 min, 1,9-bis-boc-1,5,9-triazanonane (5.30 g, 16.0 mmol) was added and the mixture was stirred for 15 min. Then additional 1,9-bis-boc-1,5,9-triazanonane (0.33 g, 1.0 mmol) was added. After complete dissolution, the reaction mixture was filtered and the solvent was evaporated at room temperature.

The residue was dissolved in 40 mL iPrOH and diluted with 320 mL MTBE. The product was precipitated over night at −20° C. The precipitate was collected by filtration through a glass filter Por. 3, and washed with 200 mL of cooled MTBE (0° C.). The product was dried in vacuo over night.

Yield 11.1 g (83%) white solid 12b.

MS: m/z 1112.86=[M+6H]⁶⁺ (calculated=1113.04).

For synthesis of compound 12c, the boc-protected compound 12b (11.1 g, 1.66 mmol) was dissolved in 40 mL of 3 M HCl in MeOH and stirred for 20 min at 45° C., then for 10 min at 55° C. For precipitation, 10 mL MeOH and 200 mL of MTBE were added and the mixture was stored for 16 h at −20° C. The precipitate was collected by filtration through a glass filter Por. 3 and washed with 200 mL of cooled MTBE (0° C.). The product was dried in vacuo over night.

Yield 9.14 g (89%) white powder 12c (HCl salt).

MS: m/z 979.45=[M+6H]⁶⁺ (calculated=979.55).

For synthesis of compound 12d, compound 12c (9.06 g, 1.47 mmol, HCl salt) and bis(pentafluorophenyl)carbonate (6.95 g, 17.6 mmol) were dissolved in 50 mL of DCM (anhydrous) and DIPEA (4.56 g, 35.3 mmol, 6.15 mL) was added at room temperature. After 10 min, 1,9-bis-boc-1,5,9-triazanonane (7.80 g, 23.5 mmol) was added and the mixture was stirred for 15 min. Then additional 1,9-bis-boc-1,5,9-triazanonane (0.49 g, 1.5 mmol) was added. After complete dissolution, the solvent was evaporated at room temperature.

The residue was dissolved in 35 mL iPrOH at 40° C. and diluted with 200 mL MTBE. The product was precipitated over night at −20° C. The precipitate was collected by filtration through a glass filter Por. 3, and washed with 200 mL of cooled MTBE (0° C.). The product was dried in vacuo over night to give 12d as a white solid.

Yield 11.6 g (90%) white solid 12d.

MS: m/z 1248.08=[M+7H]⁷⁺ (calculated=1248.27).

For synthesis of compound 12e, the boc-protected compound 12d (11.4 g, 1.31 mmol) was dissolved in 40 mL of 3 M HCl in MeOH and stirred for 20 min at 45° C., then for 10 min at 55° C. For precipitation, 10 mL MeOH and 200 mL of MTBE were added and the mixture was stored for 16 h at −20° C. The precipitate was collected by filtration through a glass filter Por. 3 and washed with 200 mL of cooled MTBE (0° C.). The product was dried in vacuo over night to give white powder 12e.

Yield 7.60 g (75%) white powder 12e (HCl salt).

MS: m/z 891.96=[M+8H]⁸⁺ (calculated=892.13).

For synthesis of compound 12f, compound 12e (7.56 g, 0.980 mmol, HCl salt) and bis(pentafluorophenyl)carbonate (9.27 g, 23.0 mmol) were dissolved in 250 mL of DCM (anhydrous) and DIPEA (6.08 g, 47.0 mmol, 8.19 mL) was added at 35° C. After 10 min, 1,9-bis-boc-1,5,9-triazanonane (5.30 g, 16.0 mmol) was added and the mixture was stirred for 15 min. Then additional 1,9-bis-boc-1,5,9-triazanonane (0.33 g, 1.0 mmol) was added. After complete dissolution, the solvent was evaporated at room temperature.

The residue was dissolved in 250 mL iPrOH at 60° C. and diluted with 1350 mL MTBE. The product was precipitated over night at −20° C. The precipitate was collected by filtration through a glass filter Por. 3, and washed with 400 mL of cooled MTBE (0° C.). The product was dried in vacuo over night to give 12f as a glassy solid.

Yield 11.1 g (83%) glassy solid 12f.

MS: m/z 1312.01=[M+10H]¹⁰⁺ (calculated=1312.21).

For synthesis of backbone reagent 12g, the boc-protected compound 12f (7.84 g, 0.610 mmol) was dissolved in 16 mL of MeOH at 37° C. and 55 mL of a precooled solution of 4 M HCl (4° C.) in dioxane was added at room temperature. The mixture was stirred without cooling for 20 min. After 20 min 110 mL of 3M HCl in MeOH was added. The solution was partitioned in 24 Falcon tubes (50 mL) and precipitated with by adding 40 mL cold MTBE (−20° C.) to each Falcon tube. After centrifugation at 3214 rcf for 1 min, the supernatant was decanted and the glassy solid was dissolved in 5 mL MeOH per Falcon tube and precipitated by adding 40 mL cold MTBE (−20° C.) to each Falcon tube again. The supernatant was discarded and the remaining solid was dried in vacuo over night.

Yield 5.74 g (87%) white glassy solid 12g (HCl salt).

MS: m/z 965.46=[M+10H]¹⁰⁺ (calculated=965.45).

### Example 13

Synthesis of Crosslinker Reagents 13d, 13g, 13k, and 13o

Crosslinker reagent 13e was prepared from azelaic acid monobenzyl ester and PEG10000 according to the following scheme:

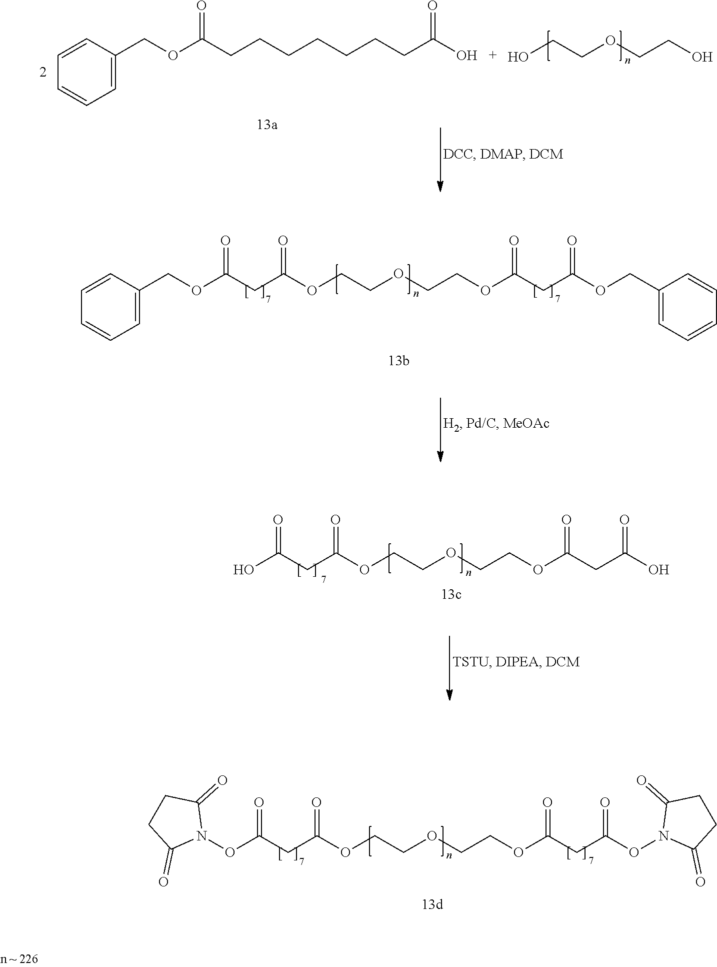

For the synthesis of azelaic acid monobenzyl ester 13a, a mixture of azelaic acid (37.6 g, 200 mmol), benzyl alcohol (21.6 g, 200 mmol), p-toluenesulfonic acid (0.80 g, 4.2 mmol), and 240 mL toluene was refluxed for 7 h in a Dean-Stark apparatus. After cooling down, the solvent was evaporated and 300 mL sat. aqueous NaHCO₃ solution were added. This mixture was extracted with 3×200 mL MTBE. The combined organic phases were dried over Na₂SO₄ and the solvent was evaporated. The product was purified on 2×340 g silica using ethyl acetate/heptane (10:90→25:75) as eluent. The eluent was evaporated and the residue was dried in vacuo over night.

Yield 25.8 g (46%) colorless oil 13a.

MS: m/z 279.16=[M+H]+(calculated=279.16).

For synthesis of compound 13b, azelaic acid monobenzyl ester 13a (3.90 g, 14.0 mmol) and PEG 10000 (40.0 g, 4.00 mmol) were dissolved in 64 mL dichloromethane and cooled with an ice bath. A solution of DCC (2.89 g, 14.0 mmol) and DMAP (0.024 g, 0.020 mmol) in 32 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 65 mL dichloromethane and diluted with 308 mL MTBE at room temperature. The mixture was stored over night at −20° C. The precipitate was collected by filtration through a glass filter Por. 3, and washed with 250 mL of cooled MTBE (−20° C.). The product was dried in vacuo over night.

Yield 40.8 g (97%) white powder 13b.

MS: m/z 835.50=[M+14H]¹⁴⁺ (calculated=835.56).

For synthesis of compound 13c, compound 13b (40.6 g, 3.86 mmol) was dissolved in methyl acetate (250 mL) and 203 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 37.2 g (93%) glassy solid 13c.

MS: m/z 882.53=[M+13H]¹³⁺ (calculated=882.51).

For synthesis of compound 13d, compound 13c (32.0 g, 3.10 mmol) and TSTU (3.73 g, 12.4 mmol) were dissolved in 150 mL dichloromethane at room temperature. Then DIPEA (1.60 g, 12.4 mmol) was added and the mixture was stirred for 1 h. The resulting suspension was filtered and the filtrate was diluted with 170 mL dichloromethane, washed with 140 mL of a solution of 750 g water/197 g NaCl/3 g NaOH. The organic phase was dried over MgSO₄ and the solvent was evaporated in vacuo.

The residue was dissolved in 200 mL toluene, diluted with 180 mL MTBE at room temperature and stored over night at −20° C. The precipitate was collected by filtration through a glass filter Por. 3, and washed with 100 mL of cooled MTBE (−20° C.). The product was dried in vacuo over night.

Yield 28.8 g (88%) white powder 13d.

MS: m/z 795.47=[M+15H]¹⁵⁺ (calculated=795.54).

Crosslinker reagent 13g was prepared from azelaic acid monobenzyl ester and PEG6000 according to the following scheme:

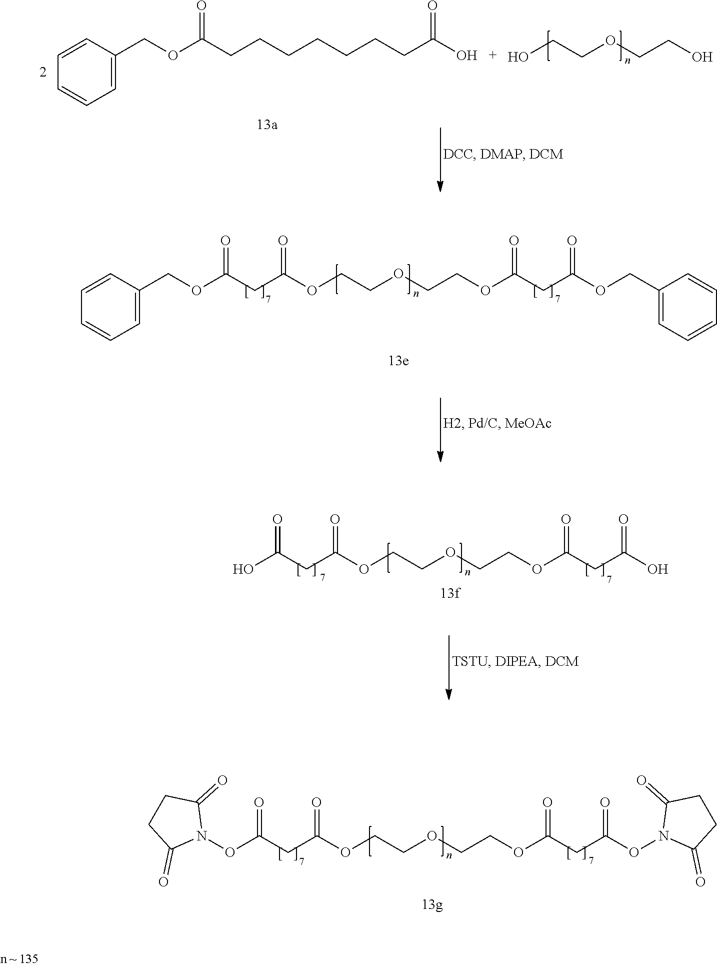

For synthesis of compound 13e, azelaic acid monobenzyl ester 13a (6.50 g, 23.3 mmol) and PEG 6000 (40.0 g, 6.67 mmol) were dissolved in 140 mL dichloromethane and cooled with an ice bath. A solution of DCC (4.81 g, 23.3 mmol) and DMAP (0.040 g, 0.33 mmol) in 40 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 70 mL dichloromethane and diluted with 300 mL MTBE at room temperature. The mixture was stored over night at −20° C. The precipitate was collected by filtration through a glass filter Por. 3, and washed with 500 mL of cooled MTBE (−20° C.). The product was dried in vacuo over night.

Yield 41.2 g (95%) white powder 13e.

MS: m/z 833.75=[M+8H]⁸⁺ (calculated=833.74).

For synthesis of compound 13f, compound 13e (41.2 g, 6.32 mmol) was dissolved in methyl acetate (238 mL) and ethanol (40 mL), then 400 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 38.4 g (96%) glassy solid 13f.

MS: m/z 750.46=[M+9H]⁹⁺ (calculated=750.56).

For synthesis of compound 13g, compound 13f (38.2 g, 6.02 mmol) and TSTU (7.25 g, mmol) were dissolved in 130 mL dichloromethane at room temperature. Then DIPEA (3.11 g, 24.1 mmol) was added and the mixture was stirred for 1 h. The resulting suspension was filtered, the filtrate was diluted with 100 mL dichloromethane and washed with 200 mL of a solution of 750 g water/197 g NaCl/3 g NaOH. The organic phase was dried over MgSO₄ and the solvent was evaporated in vacuo.

The residue was dissolved in 210 mL toluene, diluted with 430 mL MTBE at room temperature and stored over night at −20° C. The precipitate was collected by filtration through a glass filter Por. 3, and washed with 450 mL of cooled MTBE (−20° C.). The product was dried in vacuo over night.

Yield 35.8 g (91%) white powder 13g.

MS: m/z 857.51=[M+8H]⁸⁺ (calculated=857.51).

Crosslinker reagent 13k was prepared from isopropylmalonic acid monobenzyl ester and PEG10000 according to the following scheme:

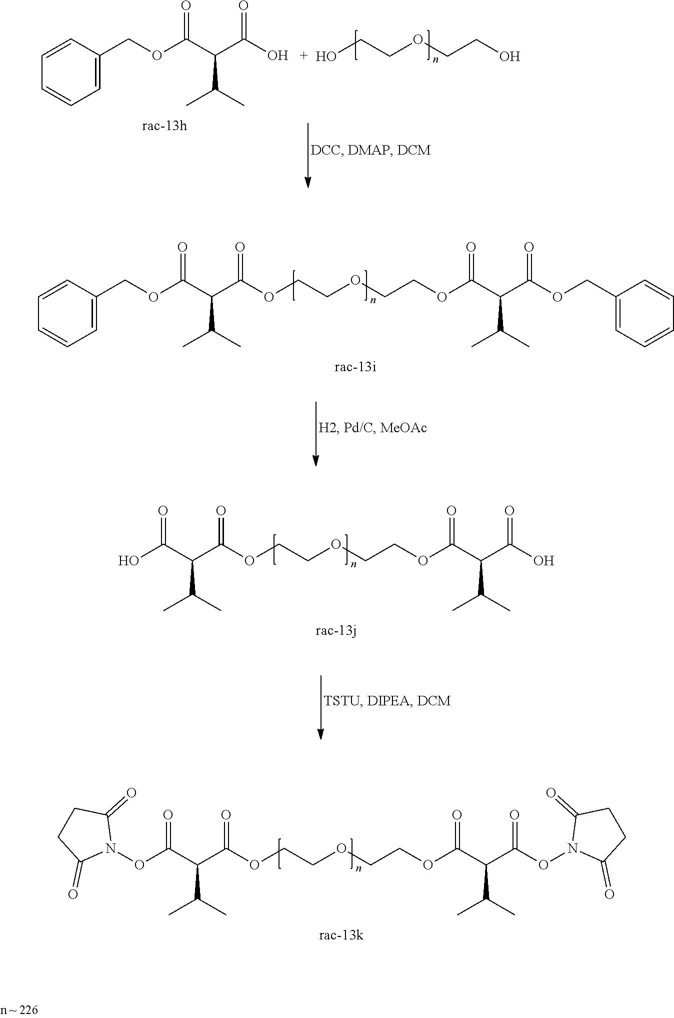

For the synthesis of isopropylmalonic acid monobenzyl ester rac-13h, isopropylmalonic acid (35.0 g, 239 mmol), benzyl alcohol (23.3 g, 216 mmol) and DMAP (1.46 g, 12.0 mmol) were dissolved in 100 mL acetonitrile. Mixture was cooled to 0° C. with an ice bath. A solution of DCC (49.4 g, 239 mmol) in 150 mL acetonitrile was added within 15 min at 0° C. The ice bath was removed and the reaction mixture was stirred over night at room temperature, then the solid was filtered off. The filtrate was evaporated at 40° C. in vacuo and the residue was dissolved in 300 mL MTBE. This solution was extracted with 2×300 mL sat. aqueous NaHCO₃ solution, then the combined aqueous phases were acidified to pH=1-3 using 6 N hydrochloric acid. The resulting emulsion was extracted with 2×300 mL MTBE and the solvent was evaporated. The combined organic phases were washed with 200 mL sat. aqueous NaCl and dried over MgSO₄. The product was purified on 340 g silica using ethyl acetate/heptane (10:90→20:80) as eluent. The eluent was evaporated and the residue was dried in vacuo over night.

Yield 9.62 g (17%) colorless oil rac-13h.

MS: m/z 237.11=[M+H]+(calculated=237.11).

For synthesis of compound 13i, isopropylmalonic acid monobenzyl ester rac-13h (945 mg, 4.00 mmol) and PEG 10000 (10.0 g, 4.00 mmol) were dissolved in 20 mL dichloromethane and cooled with an ice bath. A solution of DCC (825 mg, 4.00 mmol) and DMAP (6 mg, 0.05 mmol) in 10 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 20 mL dichloromethane and diluted with 150 mL MTBE at room temperature. The mixture was stored over night at −20° C. The precipitate was collected by filtration through a glass filter Por. 3, and washed with 500 mL of cooled MTBE (−20° C.). The product was dried in vacuo over night.

Yield 9.63 g (92%) white powder 13i.

MS: m/z 742.50=[M+16H]¹⁶⁺ (calculated=742.51).

For synthesis of compound 13j, compound 13i (3.38 g, 0.323 mmol) was dissolved in methyl acetate (100 mL) and 105 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 3.25 g (98%) glassy solid 13j.

MS: m/z 731.25=[M+16H]¹⁶⁺ (calculated=731.25).

For synthesis of compound 13k, compound 13j (3.10 g, 0.302 mmol) and TSTU (0.364 g, 1.21 mmol) were dissolved in 15 mL dichloromethane at room temperature. Then DIPEA (0.156 g, 1.21 mmol) was added and the mixture was stirred for 45 min. The resulting suspension was filtered and the filtrate was washed with 2×10 mL of a 0.5 M phosphate buffer pH=6.5. The organic phase was dried over MgSO₄ and the solvent was evaporated in vacuo. The residue was dissolved in 20 mL toluene, diluted with 10 mL MTBE at room temperature and stored over night at −20° C. The precipitate was collected by filtration through a glass filter Por. 3, and washed with 250 mL of cooled MTBE (−20° C.). The product was dried in vacuo over night.

Yield 2.66 g (84%) white powder 13k.

MS: m/z 743.37=[M+16H]¹⁶⁺ (calculated=743.38).

Crosslinker reagent rac-13o was prepared from cis-1,4-cyclohexanedicarboxylic acid and PEG10000 according to the following scheme:

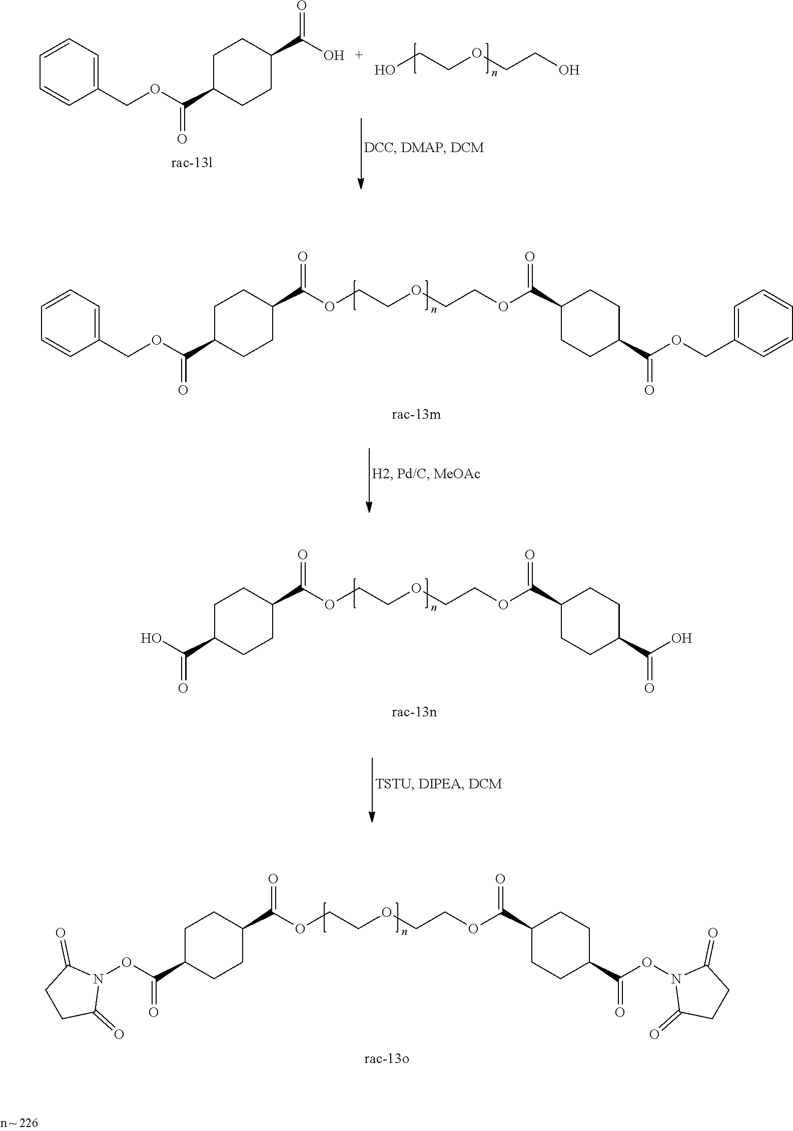

For the synthesis of cis-1,4-cyclohexanedicarboxylic acid monobenzyl ester rac-13l, cis-1,4-cyclohexanedicarboxylic acid (20.0 g, 116 mmol), benzyl alcohol (11.3 g, 105 mmol) and DMAP (710 mg, 5.81 mmol) were dissolved in 200 mL THF. Mixture was cooled to 0° C. with an ice bath. A solution of DCC (49.4 g, 239 mmol) in 100 mL THF was added within 15 min at 0° C. The ice bath was removed and the reaction mixture was stirred over night at room temperature, then the solid was filtered off. The filtrate was evaporated at 40° C. and the residue was dissolved in 300 mL MTBE. This solution was extracted with 2×300 mL sat. aqueous NaHCO₃ solution, then the combined aqueous phases were acidified to pH=1-3 using 6 N hydrochloric acid. The resulting emulsion was extracted with 2×300 mL MTBE and the solvent was evaporated. The combined organic phases were washed with 200 mL sat. aqueous NaCl and dried over MgSO₄. The product was purified on 340 g silica using ethyl acetate/heptane (10:90→20:80) as eluent. The eluent was evaporated and the colorless oily residue crystallized during drying in vacuo over night.

Yield 4.82 g (16%) colorless crystals rac-13l.

MS: m/z 263.13=[M+H]+(calculated=263.13).

For synthesis of compound 13m, cis-1,4-cyclohexanedicarboxylic acid monobenzyl ester rac-21 (2.10 g, 8.00 mmol) and PEG 10000 (20.0 g, 10.0 mmol) were dissolved in 50 mL dichloromethane and cooled with an ice bath. A solution of DCC (1.65 g, 8.00 mmol) and DMAP (0.012 g, 0.10 mmol) in 25 mL dichloromethane was added. The ice bath was removed and mixture was stirred at room temperature overnight. The resulting suspension was cooled to 0° C. and the solid was filtered off. The solvent was evaporated in vacuo.

The residue was dissolved in 55 mL dichloromethane and diluted with 300 mL MTBE at room temperature. The mixture was stored over night at −20° C. The precipitate was collected by filtration through a glass filter Por. 3, and washed with 250 mL of cooled MTBE (−20° C.). The product was dried in vacuo over night.

Yield 18.2 g (87%) white powder 13m.

MS: m/z 745.76=[M+16H]¹⁶⁺ (calculated=745.77).

For synthesis of compound 13n, compound 13m (9.00 g, 0.857 mmol) was dissolved in methyl acetate (100 mL) and 157 mg of palladium on charcoal was added. Under a hydrogen atmosphere of ambient pressure, the mixture was stirred overnight at room temperature. The reaction mixture was filtered through a pad of celite and the filtrate was evaporated and dried in vacuo over night.

Yield 8.83 g (100%) glassy solid 13n.

MS: m/z 734.50=[M+16H]¹⁶⁺ (calculated=734.50).

For synthesis of compound 13o, compound 13n (8.92 g, 0.864 mmol) and TSTU (1.04 g, 3.64 mmol) were dissolved in 35 mL dichloromethane at room temperature. Then DIPEA (0.447 g, 3.46 mmol) was added and the mixture was stirred for 45 min. The resulting suspension was filtered and the filtrate was washed with 2×10 mL of a 0.5 M phosphate buffer pH=6.5. The organic phase was dried over MgSO₄ and the solvent was evaporated in vacuo.

The residue was dissolved in 50 mL toluene, diluted with 25 mL MTBE at room temperature and stored over night at −20° C. The precipitate was collected by filtration through a glass filter Por. 3, and washed with 400 mL of cooled MTBE (−20° C.). The product was dried in vacuo over night.

Yield 7.62 g (84%) white powder 13o.

MS: m/z 702.60=[M+16H]¹⁶⁺ (calculated=702.59).

### Example 14

Preparation of hydrogel beads 14a, 14b, 14c, and 14d containing free amino groups.

In a cylindrical 250 mL reactor with bottom outlet, diameter 60 mm, equipped with baffles, an emulsion of 218 mg Cithrol™ DPHS in 100 mL undecane was stirred with an isojet stirrer, diameter 50 mm at 580 rpm, at ambient temperature. A solution of 250 mg 12a and 2205 mg 13d in 22.1 g DMSO was added and stirred for 10 min at RT to form a suspension. 1.1 mL TMEDA were added to effect polymerization. The mixture was stirred for 16 h. 1.7 mL of acetic acid were added and then after 10 min 100 mL of a 15 wt % solution of sodium chloride in water was added. After 10 min, the stirrer was stopped and phases were allowed to separate. After 2 h the aqueous phase containing the hydrogel was drained.

For bead size fractionation, the water-hydrogel suspension was diluted with 40 mL ethanol and wet-sieved on 125, 100, 75, 63, 50, 40, and 32 μm steel sieves using a Retsch AS200 control sieving machine for 15 min. Sieving amplitude was 1.5 mm, water flow 300 mL/min. Bead fractions that were retained on the 63 and 75 μm sieves were pooled and washed 3 times with 0.1% AcOH, 10 times with ethanol and dried for 16 h at 0.1 mbar to give 670 mg of 14a as a white powder.

Amino group content of the hydrogel was determined to be 0.145 mmol/g by conjugation of a fmoc-amino acid to the free amino groups on the hydrogel and subsequent fmoc-determination.

14b was prepared as described for 14a except for the use of 350 mg 12a, 2548 mg 13g, 26.1 g DMSO, 257 mg Cithrol™ DPHS, 1.5 mL TMEDA, and 2.4 mL acetic acid, yielding 550 mg 14b as a white powder, free amino groups 0.120 mmol/g. 14c was prepared as described for 14a except for the use of 250 mg 12a, 3019 mg rac-13k, 32.7 g DMSO, 290 mg Cithrol™ DPHS, 1.1 mL ml TMEDA, and 1.7 mL acetic acid, yielding 770 mg 13c as a white powder, free amino groups 0.126 mmol/g. 14d was prepared as described for 14a except for the use of 250 mg 12a, 2258 mg rac-13o, 22.6 g DMSO, 222 mg Cithrol™ DPHS, 1.1 mL ml TMEDA, and 1.7 mL acetic acid, yielding 186 mg 14d as a white powder, free amino groups 0.153 mmol/g.

### Example 15

Synthesis of Linker Reagent 15c

Linker reagent 15c was synthesized according to the following scheme:

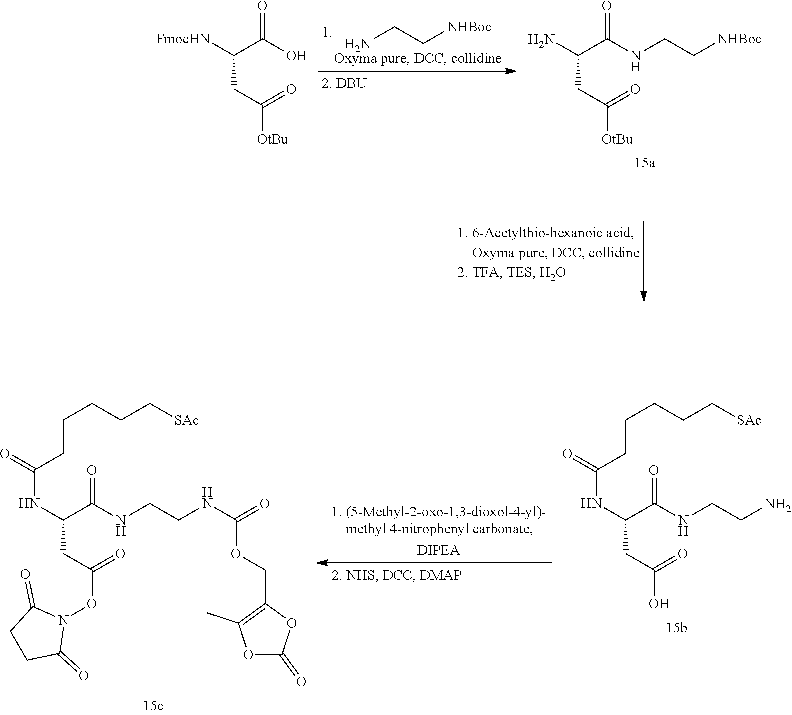

Synthesis of 15a:

Fmoc-L-Asp(OtBu)-OH (1.00 g, 2.43 mmol) was dissolved with DCC (0.70 g, 3.33 mmol) in DCM (25 mL). Oxyma pure (0.51 g, 3.58 mmol) and collidine (0.50 mL, 3.58 mmol) were added in one portion and a solution of N-Boc-ethylenediamine (0.41 g, 2.56 mmol) in DCM (15 mL) was added slowly. After stirring the mixture for 90 min at RT the formed precipitate was filtered off and the filtrate washed with aqueous HCl (0.1 M, 50 mL). The aqueous layer was extracted with DCM (2×20 mL) and the combined organic fractions were washed with sat. aqueous NaHCO₃ (3×25 mL) and brine (1×50 mL), dried over Na₂SO₄, filtered and concentrated in vacuo. The crude solid was purified by flash chromatography. The intermediate N-boc-N′—(N-fmoc-4-tert.-butyl-L-aspartoyl)-ethylenediamine was obtained as white solid (0.98 g, 1.77 mmol, 73%).

MS: m/z 554.29=[M+H]⁺, (calculated=554.29).

N-boc-N′—(N-fmoc-4-tert.-butyl-L-aspartoyl)-ethylenediamine (0.98 g, 1.77 mmol) was dissolved in THF (15 mL), DBU (0.31 mL) was added and the solution was stirred for 12 min at RT. The reaction was quenched with AcOH (0.5 ml), concentrated in vacuo and the residue purified by flash chromatography to give 15a (0.61 g, 1.77 mmol, 73% over 2 steps) as white solid.

MS: m/z 332.38=[M+H]⁺, (calculated=332.22).

Synthesis of 15b:

6-Acetylthiohexanoic acid (0.37 g, 1.95 mmol) was dissolved in DCM (19.5 mL) and Oxyma pure (0.35 g, 2.48 mmol) and DCC (0.40 g, 1.95 mmol) added in one portion. The solution was stirred for 30 min at RT, filtered, and the filtrate added to a solution of 15a (0.61 g, 1.77 mmol) in DCM (10.5 mL). DIPEA (0.46 mL, 2.66 mmol) was added to the solution and the reaction stirred for 2 h at RT. The solution was washed with aqueous H₂SO₄ (0.1 M, 2×30 mL), sat. aqueous NaHCO₃ (2×20 mL) and brine (1×20 mL). The organic layer was dried over Na₂SO₄, filtered and concentrated in vacuo. The crude material was purified by flash chromatography to give N-boc-N′—(N-6-acetylthiohexyl-4-tert.-butyl-L-aspartoyl)-ethylenediamine (0.65 g, 1.30 mmol, 73% over 2 steps) as white solid.

MS: m/z 504.27=[M+H]⁺, (calculated=504.28).

N-boc-N′—(N-6-Acetylthiohexyl-4-tert.-butyl-L-aspartoyl)-ethylenediamine (0.60 g, 1.18 mmol) was dissolved in TFA (5 mL) and TES (0.13 mL) and water (0.13 ml) were added. The mixture was stirred for 30 min at RT. TFA was removed in a stream of N₂, and crude 15b dissolved in H2O/ACN 1:1 and purified by RP-HPLC.

Yield: 0.39 g, 0.85 mmol (TFA salt), 72%.

MS: m/z 348.25=[M+H]⁺, (calculated=348.16).

Synthesis of 15c:

15b (TFA salt, 0.38 g, 0.80 mmol) was dissolved in DMF (5 mL) and (5-methyl-2-oxo-1,3-dioxol-4-yl)-methyl 4-nitrophenyl carbonate (0.26 g, 0.88 mmol) and DIPEA (0.28 mL, 1.6 mmol) were added. The resulting suspension was diluted with DCM (5 mL) and stirred for 3 h at RT. More DIPEA (0.28 mL 1.6 mmol) was added and stirring continued for 2 h. DCM was concentrated in vacuo, the residue diluted with H2O/ACN 3:1 and purified by RP-HPLC to give N-(5-methyl-2-oxo-1,3-dioxol-4-yl)-methyl-oxocarbonyl-N′—(N-6-acetylthiohexyl-L-aspartyl)-ethylenediamine (0.31 g, 0.62 mmol, 77%) as colorless oil.

MS: m/z 504.16=[M+H]⁺, (calculated=504.17).

N-(5-methyl-2-oxo-1,3-dioxol-4-yl)-methyl oxocarbonyl-N′—(N-6-acetylthiohexyl-L-aspartyl)-ethylene-diamine (150 mg, 0.30 mmol) was dissolved in DCM (17.5 mL) and NHS (41 mg, 0.36 mmol), DCC (74 mg, 0.36 mmol) and DMAP (4 mg, 0.03 mmol) were added in one portion. The reaction was stirred for 1 h at RT and the resulting suspension filtered. The precipitate was washed with a small amount of DCM and the combined filtrates concentrated in vacuo. 15c was purified by RP-HPLC to give a colorless oil (144 mg, 0.24 mmol, 80%).

MS: m/z 601.18=[M+H]⁺, (calculated=601.18).

### Example 16

Preparation of Maleimide Functionalized Hydrogel Beads 16a

259.3 mg of dry hydrogel beads 14a was incubated for 15 min in 10 mL 1% n-propylamine in NMP and subsequently washed two times with 1% n-propylamine in NMP and two times with 2% DIPEA in NMP. 171 mg of maleimide-NH-PEG12-PFE was dissolved in 1 mL NMP and added to the washed hydrogel beads 14a. The hydrogel suspension was incubated for 2 h at room temperature. Resulting maleimide functionalized hydrogel beads 16a were washed five times each with NMP, 20 mM succinate, 1 mM Na₂EDTA, 0.01% Tween20, pH 3.0, water, and with 0.1% acetic acid, 0.01% Tween20.

### Example 17

Synthesis of transient Lucentis-linker-hydrogel prodrug 17c 4.6 mg Lucentis (depicted in the scheme below as Lucentis-NH₂) (460 μL of 10 mg/mL Lucentis in 10 mM histidine, 10 wt % α,α-trehalose, 0.01% Tween20, pH 5.5) was buffer exchanged to 10 mM sodium phosphate, 2.7 mM potassium chloride, 140 mM sodium chloride, pH 7.4 and the concentration of Lucentis was adjusted to 16.4 mg/mL. 6 mg of Linker reagent 15c was dissolved in 100 μL DMSO to yield a concentration of 100 mM. 1 molar equivalent of linker reagent 15c relative to the amount of Lucentis was added to the Lucentis solution. The reaction mixture was mixed carefully and incubated for 5 min at room temperature. Subsequently, 2 additional molar equivalents of linker reagent 15c were added to the Lucentis solution in 1 molar equivalent steps and after addition of each equivalent the reaction mixture was incubated for 5 min at room temperature yielding a mixture of unmodified Lucentis and the protected Lucentis-linker monoconjugate 17a.

The pH of the reaction mixture was adjusted to pH 6.5 by addition of 1 M sodium citrate, pH 5.0 and Na₂EDTA was added to a final concentration of 5 mM. To remove the protecting groups of 17a 0.5 M NH₂OH (dissolved in 10 mM sodium citrate, 140 mM sodium chloride, 5 mM Na₂EDTA, pH 6.5) was added to a final concentration of 45 mM and the deprotection reaction was incubated at room temperature for 4 h yielding the Lucentis-linker monoconjugate 17b. The mixture of Lucentis and Lucentis-linker monoconjugate 17b was buffer exchanged to 10 mM sodium phosphate, 2.7 mM potassium chloride, 140 mM sodium chloride, 5 mM Na₂EDTA, 0.01% Tween 20, pH 6.5 and the overall concentration of the two Lucentis species was adjusted to 11.8 mg/mL. The content of Lucentis-linker monoconjugate 17b in the mixture was 20% as determined by ESI-MS.

4 mg of the Lucentis/Lucentis-linker monoconjugate 17b mixture in 10 mM sodium phosphate, 2.7 mM potassium chloride, 140 mM sodium chloride, 5 mM Na₂EDTA, 0.01% Tween 20, pH 6.5 were added to 1 mg of maleimide functionalized hydrogel beads 16a and incubated overnight at room temperature yielding transient Lucentis-linker-hydrogel prodrug 17c.

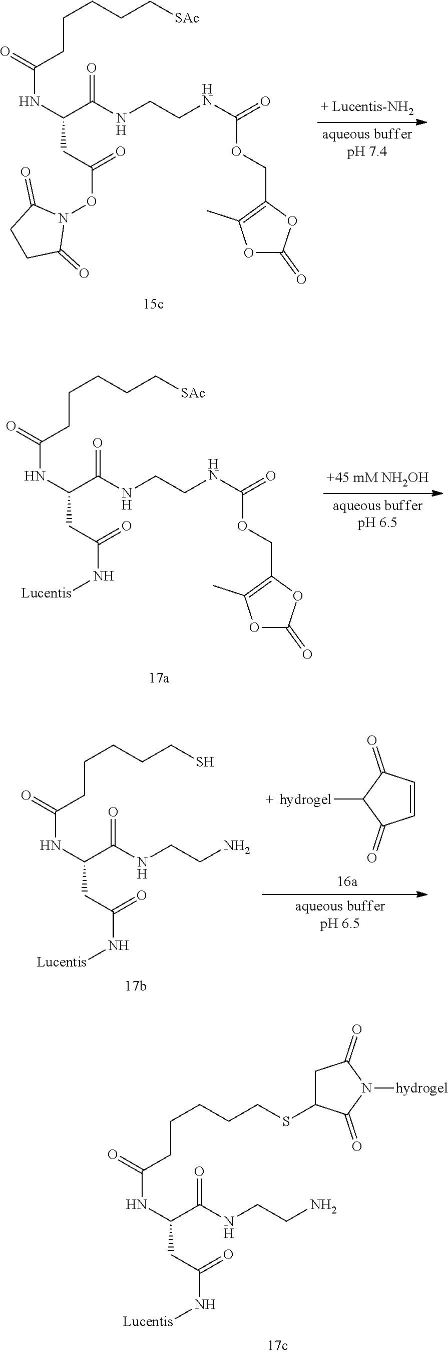

### Example 18

In Vitro Release Kinetics—Determination of In Vitro Half-Life

Lucentis-linker-hydrogel prodrug 17c (containing approximately 1 mg Lucentis) was washed five times with 60 mM sodium phosphate, 3 mM Na₂EDTA, 0.01% Tween20, pH 7.4 and finally suspended in 1 mL of the aforementioned buffer. The suspension was incubated at 37° C. The buffer of the suspension was exchanged after different time intervals and analyzed by HPLC-SEC at 220 nm. Peaks corresponding to liberated Lucentis were integrated and the total of liberated Lucentis was plotted against total incubation time. Curve fitting software was applied to determine first-order cleavage rates.

### Abbreviations

Ac acetyl
ACN acetonitrile
AcOH acetic acid
AcOEt ethyl acetate
Asp aspartate
Bn benzyl
Boc t-butyloxycarbonyl
DBU 1,3-diazabicyclo[5.4.0]undecene
DCC N,N-dicyclohexylcarbodiimid
DCM dichloromethane
DIPEA diisopropylethylamine
DMAP dimethylamino-pyridine
DMF N,N-dimethylformamide
DMSO dimethylsulfoxide
DTT DL dithiotreitol
EDC 1-Ethyl-3-(3-dimethylaminopropyl)carbodiimid
EDTA ethylenediaminetetraacetic acid
eq stoichiometric equivalent
EtOH ethanol
Fmoc 9-fluorenylmethoxycarbonyl
HPLC high performance liquid chromatography
HOBt N-hydroxybenzotriazole
iPrOH 2-propanol
LCMS mass spectrometry-coupled liquid chromatography
Mal 3-maleimido propyl
Maleimide-NH-PEG12-PFE N-(3-maleimidopropyl)-39-amino-4,7,10,13,16,19,22,25,28,31,34,37-dodecaoxa-nonatriacontanoic acid pentafluorophenyl ester
Mal-PEG6-NHS N-(3-maleimidopropyl)-21-amino-4,7,10,13,16,19-hexaoxa-heneicosanoic acid NHS ester
Me methyl
MeOAc methyl acetate
MeOH methanol
Mmt 4-methoxytrityl
MS mass spectrum/mass spectrometry
MTBE methyl tert.-butyl ether
MW molecular mass
NHS N-hydroxy succinimide
Oxyma Pure ethyl 2-cyano-2-(hydroxyimino)acetate
PEG poly(ethylene glycol)
PyBOP benzotriazole-1-yl-oxy-tris-pyrrolidino-phosphonium hexafluorophosphate
RP-HPLC reversed-phase high performance liquid chromatography
rpm rounds per minute
RT room temperature
SEC size exclusion chromatography
tBu tert.-butyl
TAN 1,5,9-triazanonane
TCEP tris(2-carboxyethyl)phosphine hydrochloride
TES triethylsilane
TFA trifluoroacetic acid
THE tetrahydrofurane
TMEDA N,N,N′N′-tetramethylethylene diamine
Trt triphenylmethyl, trityl
TSTU O—(N-succinimidyl)-N,N,N,N-tetramethyluronium
tetrafluoroborate
UPLC ultra performance liquid chromatography
V volume

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

1. A method of preventing, diagnosing and/or treating an ocular condition, wherein said method comprises:
the step of administering a therapeutically effective amount of a pharmaceutical composition comprising a hydrogel-linked prodrug to a patient in need thereof.

2. The method of claim 1;
wherein the pharmaceutical composition is administered via intraocular injection.

3. The method of claim 1;
wherein the ocular condition is an anterior ocular condition or a posterior ocular condition.

4. The method of claim 3;
wherein the anterior ocular condition is selected from the group comprising aphakia, pseudophakia, astigmatism, blepharospasm, cataract, conjunctival diseases, conjunctivitis, corneal diseases, corneal ulcer, dry eye syndromes, eyelid diseases, lacrimal apparatus diseases, lacrimal duct obstruction, myopia, presbyopia, pupil disorders, refractive disorders, glaucoma, and strabismus.

5. The method of claim 3;
wherein the posterior ocular condition is selected from the group consisting of:
acute macular neuroretinopathy;
Behcet's disease;
choroidal neovascularization;
diabetic uveitis;
histoplasmosis;
infections, including fungal-caused and viral-caused infections;
macular degeneration, including acute macular degeneration, non-exudative age related macular degeneration, and exudative age related macular degeneration;
edema, including macular edema, cystoid macular edema, and diabetic macular edema;
multifocal choroiditis;
ocular trauma which affects a posterior ocular site or location; ocular tumors;
retinal disorders, including central retinal vein occlusion, diabetic retinopathy, proliferative vitreoretinopathy (PVR), retinal arterial occlusive disease, retinal detachment, and uveitic retinal disease;
sympathetic opthalmia;
Vogt Koyanagi-Harada (VKH) syndrome;
uveal diffusion;
a posterior ocular condition caused by or influenced by an ocular laser treatment; and
posterior ocular conditions caused by or influenced by a photodynamic therapy, photocoagulation, radiation retinopathy, epiretinal membrane disorders, branch retinal vein occlusion, anterior ischemic optic neuropathy, nonretinopathy diabetic retinal dysfunction, retinitis pigmentosa, glaucoma, or by a combination thereof.

6. The method of claim 1;
wherein the pharmaceutical composition is contained in a container suited for engagement with an injection device.

7. The method of claim 1;
wherein the hydrogel is a biodegradable hydrogel.

8. The method of claim 1;
wherein the hydrogel is a PEG-based hydrogel.

9. The method of claim 1;
wherein the hydrogel-linked prodrug is bead-shaped.

10. The method of claim 9;
wherein the beads have a diameter of 1 to 1000 μm.

11. The method of claim 1;
wherein the hydrogel is obtained by a process comprising the steps of:
(a) providing a mixture comprising:
(a-i) at least one backbone reagent, wherein the at least one backbone reagent has a molecular weight ranging from 1 to 100 kDa, and comprises at least three amines (—NH₂ and/or —NH—);
(a-ii) at least one PEG-based crosslinker reagent, wherein the at least one PEG-based crosslinker reagent has a molecular weight ranging from 6 to 40 kDa, the at least one PEG-based crosslinker reagent comprising:
(i) at least two carbonyloxy groups (—(C═O)—O— or —O—(C═O)—);
(ii) at least two activated functional end groups selected from the group consisting of activated ester groups, activated carbamate groups, activated carbonate groups and activated thiocarbonate groups; and
(iii) at least 70% PEG; and
(a-iii) a first solvent and at least a second solvent, which second solvent is immiscible in the first solvent;
wherein a weight ratio of the at least one backbone reagent to the at least one PEG-based crosslinker reagent is from 1:99 to 99:1;
(b) polymerizing the mixture of step (a) in a suspension polymerization to a hydrogel; and
(c) optionally working-up the hydrogel.

12. The method of claim 11;
wherein the mixture of step (a) further comprises a detergent.

13. The method of claim 11;
wherein the polymerization in step (b) is initiated by adding a base.

14. The method of claim 11;
wherein the mixture of step (a) is an emulsion.

15. The method of claim 11;
wherein the at least one backbone reagent is selected from the group consisting of:
(i) a compound of formula (I):
B(-(A⁰)ₓ₁-(SP)ₓ₂-A¹-P-A²-Hyp¹)ₓ (I);
wherein:
B is a branching core;
SP is a spacer moiety selected from the group consisting of C₁₋₆ alkyl, C₂₋₆ alkenyl and C₂₋₆ alkynyl;
P is a PEG-based polymeric chain comprising at least 80% PEG, preferably at least 85% PEG, more preferably at least 90% PEG and most preferably at least 95% PEG;
Hyp¹ is a moiety comprising at least one amine selected from the group consisting of —NH₂ and —NH—;
x is an integer from 3 to 16;
x1 and x2 are independently of each other 0 or 1, provided that x1 is 0 if x2 is 0;
A⁰, A¹, and A² are independently of each other selected from the group consisting of: 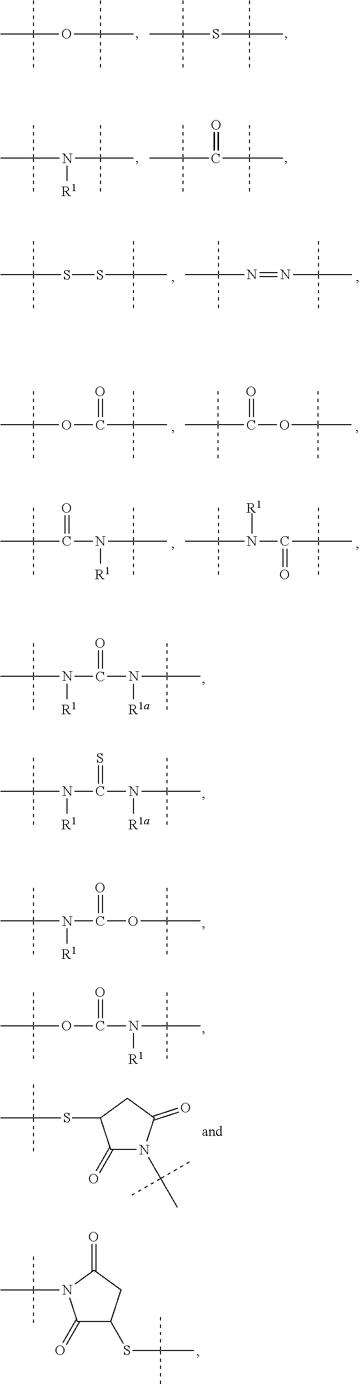
wherein R¹ and R^{1a} are independently of each other selected from H and C₁₋₆ alkyl;
(ii) a compound of formula (II):
Hyp²-A³-P-A⁴-Hyp³ (II);
wherein:
P is defined as above in the compound of formula (I);
Hyp² and Hyp³ are independently of each other a polyamine comprising at least two amines selected from the group consisting of —NH₂ and —NH—; and
A³ and A⁴ are independently selected from the group consisting of: 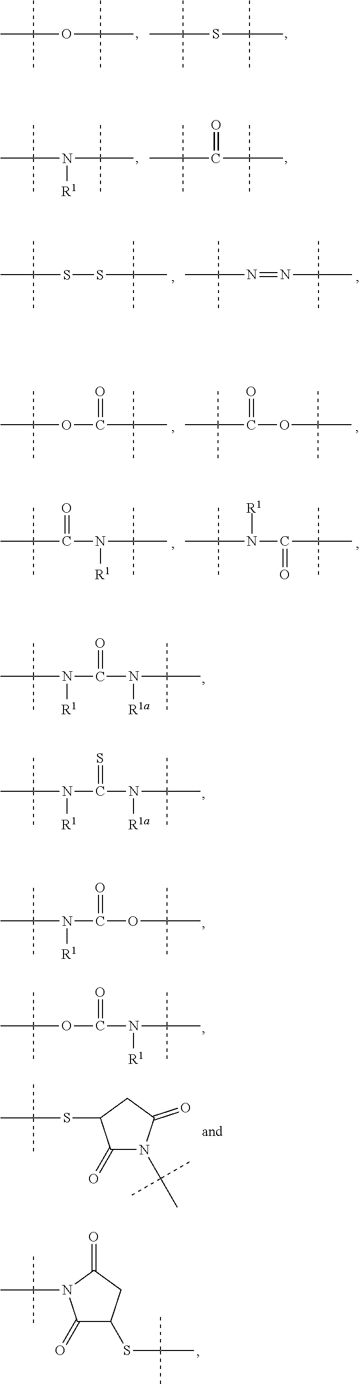
wherein R¹ and R^{1a} are independently of each other selected from H and C₁₋₆ alkyl;
(iii) a compound of formula (III):
P¹-A⁵-Hyp⁴ (III);
wherein:
P¹ is a PEG-based polymeric chain comprising at least 80% PEG;
Hyp⁴ is a polyamine comprising at least three amines selected from the group consisting of —NH₂ and —NH—; and
A⁵ is selected from the group consisting of: 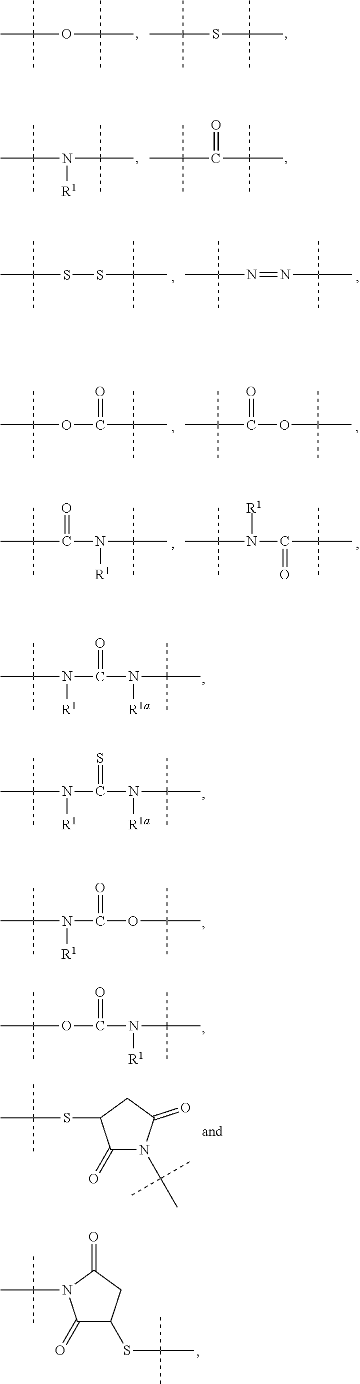
wherein R¹ and R^{1a} are independently of each other selected from H and C₁₋₆ alkyl; and
(iv) a compound of formula (IV):
T¹-A⁶-Hyp⁵ (IV);
wherein:
Hyp⁵ is a polyamine comprising at least three amines selected from the group consisting of —NH₂ and —NH—; and
A⁶ is selected from the group consisting of: 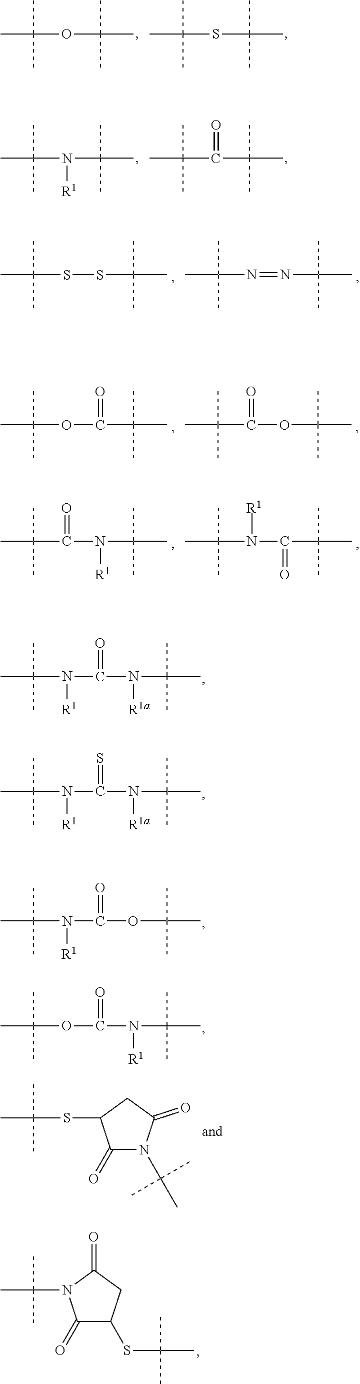
wherein R¹ and R^{1a} are independently of each other selected from H and C₁₋₆ alkyl; and
T¹ is selected from the group consisting of C₁₋₅₀ alkyl, C₂₋₅₀ alkenyl and C₂₋₅₀ alkynyl, which fragment is optionally interrupted by at least one group selected from the group consisting of —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl, and naphthyl.

16. The method of claim 15;
wherein Hyp¹, Hyp², Hyp³, Hyp⁴, and Hyp⁵ are selected from the group consisting of:
(i) a moiety of formula (e-i): 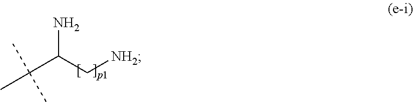
wherein:
p1 is an integer from 1 to 5; and
the dashed line indicates attachment to A² if the backbone reagent has a structure of formula (I), and to A³ or A⁴ if the backbone reagent has the structure of formula (II);
(ii) a moiety of formula (e-ii): 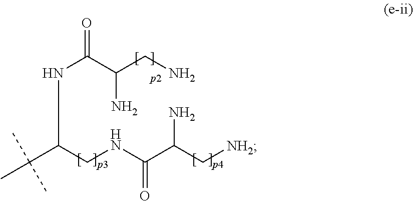
wherein:
p2, p3, and p4 are identical or different and each is independently of the others an integer from 1 to 5; and
the dashed line indicates attachment to A² if the backbone reagent has a structure of formula (I), to A³ or A⁴ if the backbone reagent has a structure of formula (II), to A⁵ if the backbone reagent has a structure of formula (III), and to A⁶ if the backbone reagent has a structure of formula (IV);
(iii) a moiety of formula (e-iii): 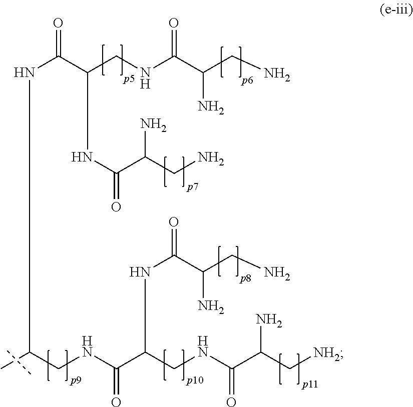
wherein:
p5 to p11 are identical or different and each is independently of the others an integer from 1 to 5; and
the dashed line indicates attachment to A² if the backbone reagent is of formula (I), to A³ or A⁴ if the backbone reagent is of formula (II), to A⁵ if the backbone reagent is of formula (III), and to A⁶ if the backbone reagent is of formula (IV);
(iv) a moiety of formula (e-iv): 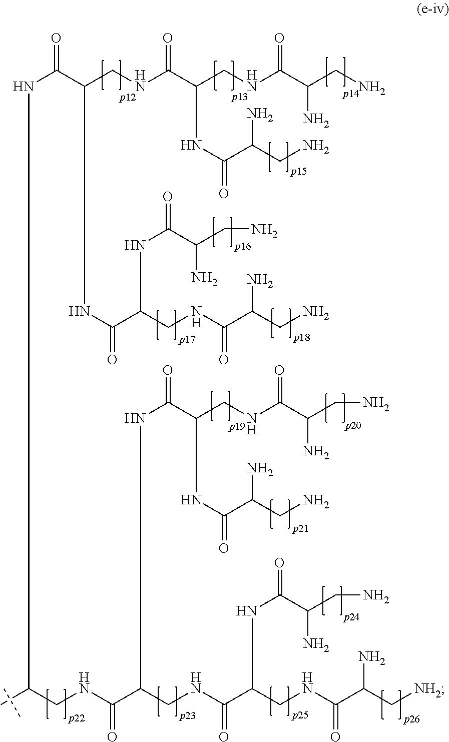
wherein:
p12 to p26 are identical or different and each is independently of the others an integer from 1 to 5; and
the dashed line indicates attachment to A² if the backbone reagent has a structure of formula (I), to A³ or A⁴ if the backbone reagent has a structure of formula (II), to A⁵ if the backbone reagent has a structure of formula (III), and to A⁶ if the backbone reagent has a structure of formula (IV);
(v) a moiety of formula (e-v): 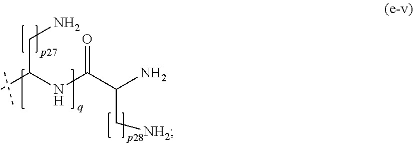
wherein:
p27 and p28 are identical or different and each is independently of the other an integer from 1 to 5;
q is an integer from 1 to 8; and
the dashed line indicates attachment to A² if the backbone reagent has a structure of formula (I), to A³ or A⁴ if the backbone reagent has a structure of formula (II), to A⁵ if the backbone reagent has a structure of formula (III) and to A⁶ if the backbone reagent has a structure of formula (IV);
(vi) a moiety of formula (e-vi): 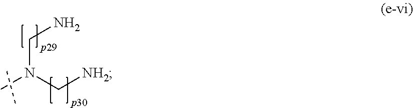
wherein:
p29 and p30 are identical or different and each is independently of the other an integer from 2 to 5; and
the dashed line indicates attachment to A² if the backbone reagent has the structure of formula (I), to A³ or A⁴ if the backbone reagent has the structure of formula (II), to A⁵ if the backbone reagent has the structure of formula (III), and to A⁶ if the backbone reagent has the structure of formula (IV);
(vii) a moiety of formula (e-vii): 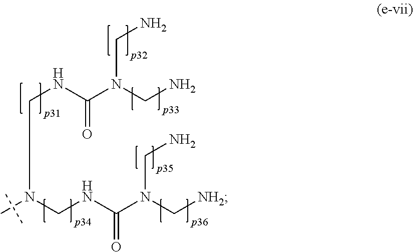
wherein:
p31 to p36 are identical or different and each is independently of the others an integer from 2 to 5; and
the dashed line indicates attachment to A² if the backbone reagent has a structure of formula (I), to A³ or A⁴ if the backbone reagent has a structure of formula (II), to A⁵ if the backbone reagent has a structure of formula (III), and to A⁶ if the backbone reagent has a structure of formula (IV);
(viii) a moiety of formula (e-viii): 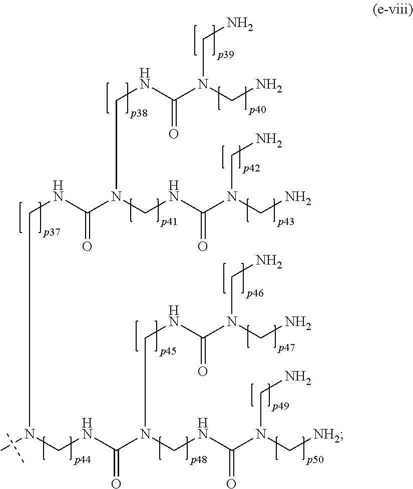
wherein:
p37 to p50 are identical or different and each is independently of the others an integer from 2 to 5; and
the dashed line indicates attachment to A² if the backbone reagent has a structure of formula (I), to A³ or A⁴ if the backbone reagent has a structure of formula (II), to A⁵ if the backbone reagent has a structure of formula (III), and to A⁶ if the backbone reagent has a structure of formula (IV); and
(ix) a moiety of formula (e-ix): 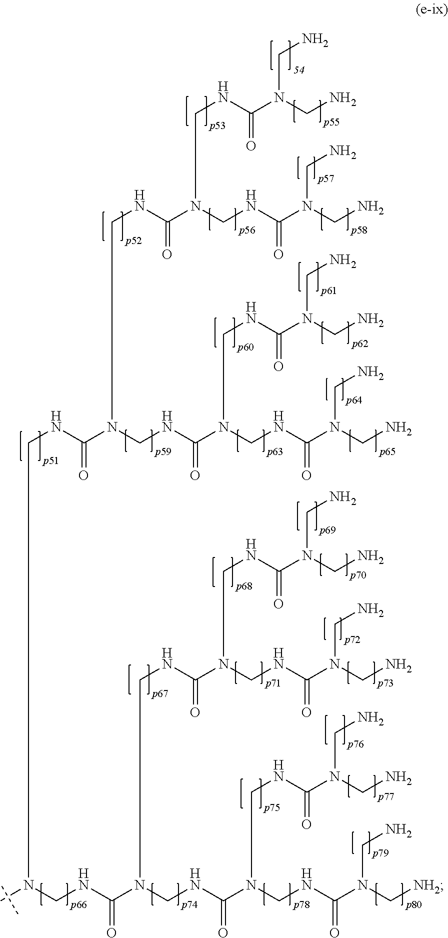
wherein:
p51 to p80 are identical or different and each is independently of the others an integer from 2 to 5; and
the dashed line indicates attachment to A² if the backbone reagent has a structure of formula (I), to A³ or A⁴ if the backbone reagent has a structure of formula (II), to A⁵ if the backbone reagent has a structure of formula (III), and to A⁶ if the backbone reagent has a structure of formula (IV); and
wherein the moieties (e-i) to (e-v) may at each chiral center be in either R- or S-configuration.

17. The method of claim 15;
wherein the backbone reagent is a compound of formula (I).

18. The method of claim 15;
wherein the branching core B is selected from the following structures: 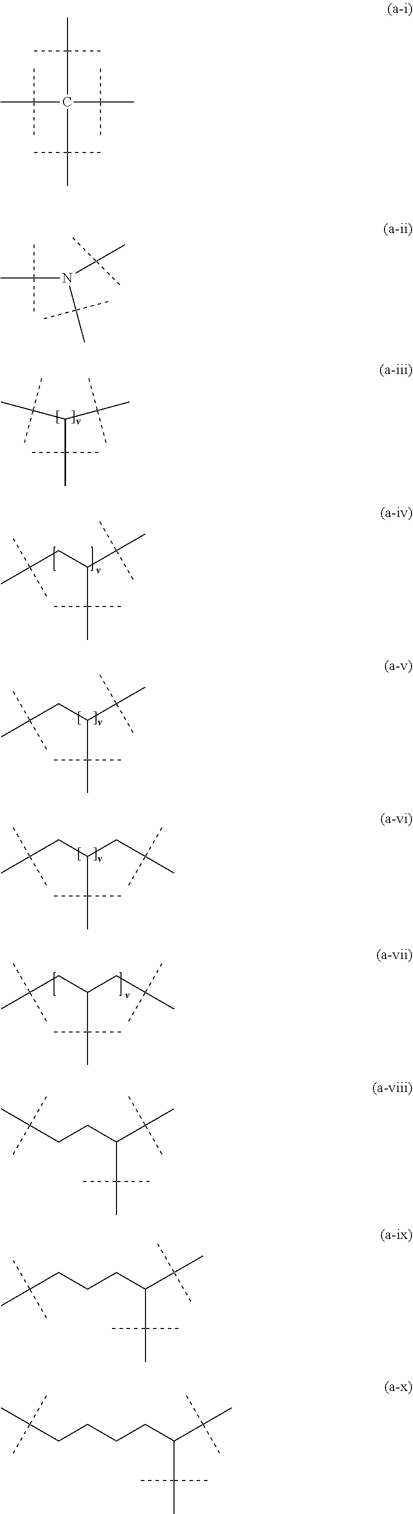 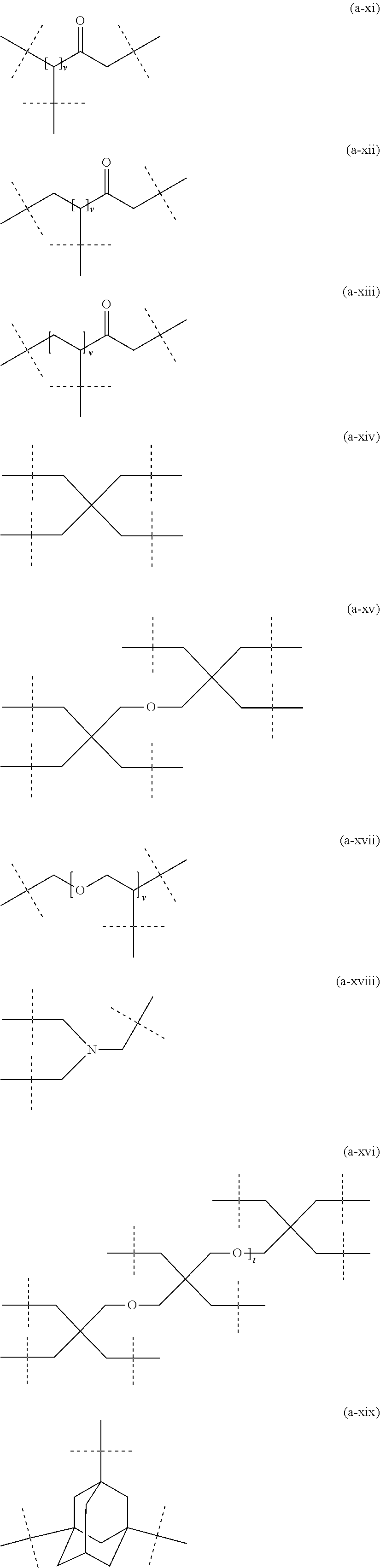 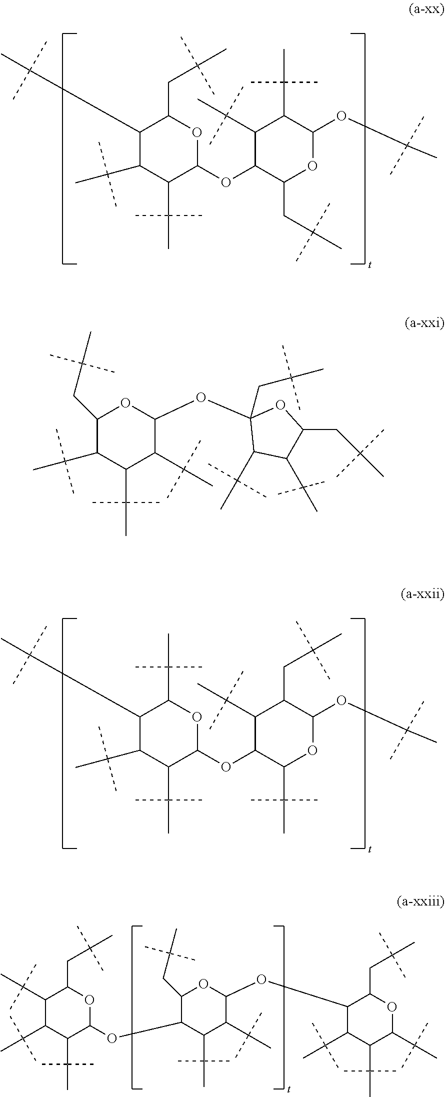
wherein:
dashed lines indicate attachment to A⁰ or, if x1 and x2 are both 0, to A¹;
t is 1 or 2; and
v is 1, 2, 3, 4, 5, 6, 7, 8, 9, 10, 11, 12, 13, or 14.

19. The method of claim 18;
wherein B is of formula (a-xiv).

20. The method of claim 15;
wherein A⁰ is:

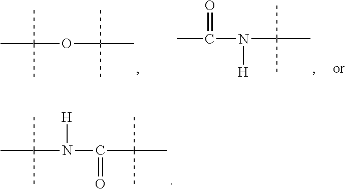

21. The method of claim 15;
wherein x1 and x2 are 0.

22. The method of claim 15;
wherein P has the structure of formula (c-i): 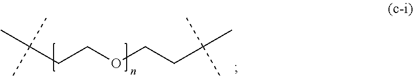
wherein n ranges from 6 to 900.

23. The method of claim 16;
wherein the at least one backbone reagent is of formula (I); and
wherein the moiety -A²-Hyp¹ is a moiety of the formula: 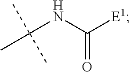
wherein:
the dashed line indicates attachment to P; and
E¹ is selected from formulas (e-i) to (e-ix).

24. The method of claim 11;
wherein the backbone reagent has the following formula: 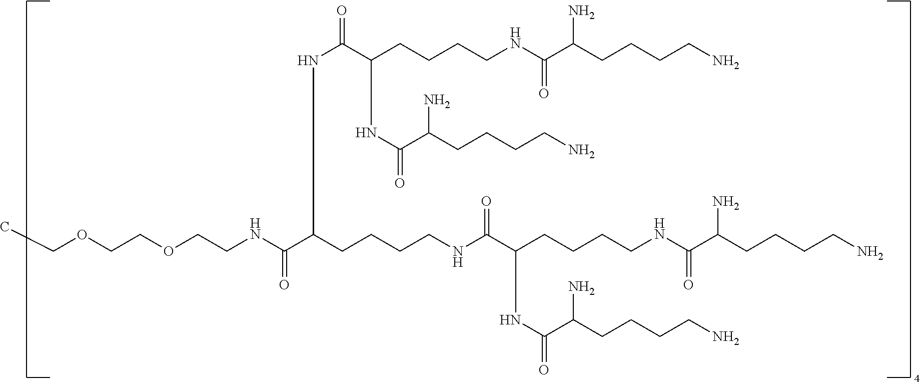
wherein:
n ranges from 10 to 40.

25. The method of claim 11;
wherein the backbone reagent is present in the form of its acidic salt.

26. The method of claim 11;
wherein the crosslinker reagent is a compound of formula (V): 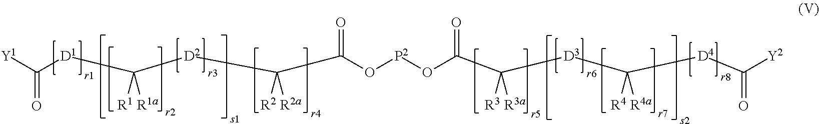
wherein:
D¹, D², D³, and D⁴ are identical or different and each is independently of the others selected from the group comprising O, NR⁵, S and CR⁵R^{5a};
R¹, R^{1a}, R², R^{2a}, R³, R^{3a}, R⁴, R^{4a}, R⁵, and R^{5a} are identical or different and each is independently of the others selected from the group comprising H and C₁₋₆ alkyl;
where, optionally, one or more of the pair(s) R¹/R^{1a}, R²/R^{2a}, R³/R^{3a}, R⁴/R^{4a}, R¹/R², R³/R⁴, R^{1a}/R², and R^{3a}/R^{4a} form a chemical bond or are joined together with the atom to which they are attached to form a C₃₋₈ cycloalkyl or to form a ring A or are joined together with the atom to which they are attached to form a 4-membered to 7-membered heterocyclyl or 8-membered to 11-membered heterobicyclyl or adamantyl;
A is selected from the group consisting of phenyl, naphthyl, indenyl, indanyl, and tetralinyl;
P2 is: 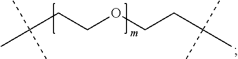
where m ranges from 120 to 920;
r1, r2, r7, and r8 are independently 0 or 1;
r3 and r6 are independently 0, 1, 2, 3, or 4;
r4 and r5 are independently 1, 2, 3, 4, 5, 6, 7, 8, 9, or 10;
s1 and s2 are independently 1, 2, 3, 4, 5, or 6;
Y¹ and Y² are identical or different and each is independently of the other selected from formulas (f-i) to (f-vi): 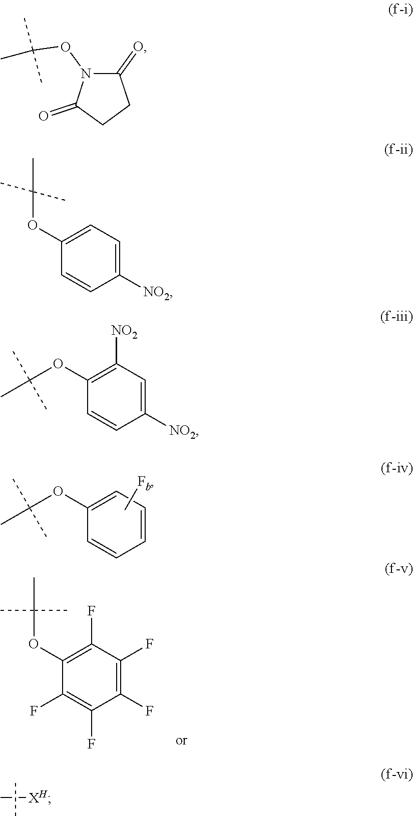
wherein:
the dashed lines indicate attachment to the rest of the molecule;
b is 1, 2, 3, or 4; and
X^{H} is Cl, Br, I, or F.

27. The method of claim 11;
wherein the crosslinker reagent is of formula (V-I) to (V-53): 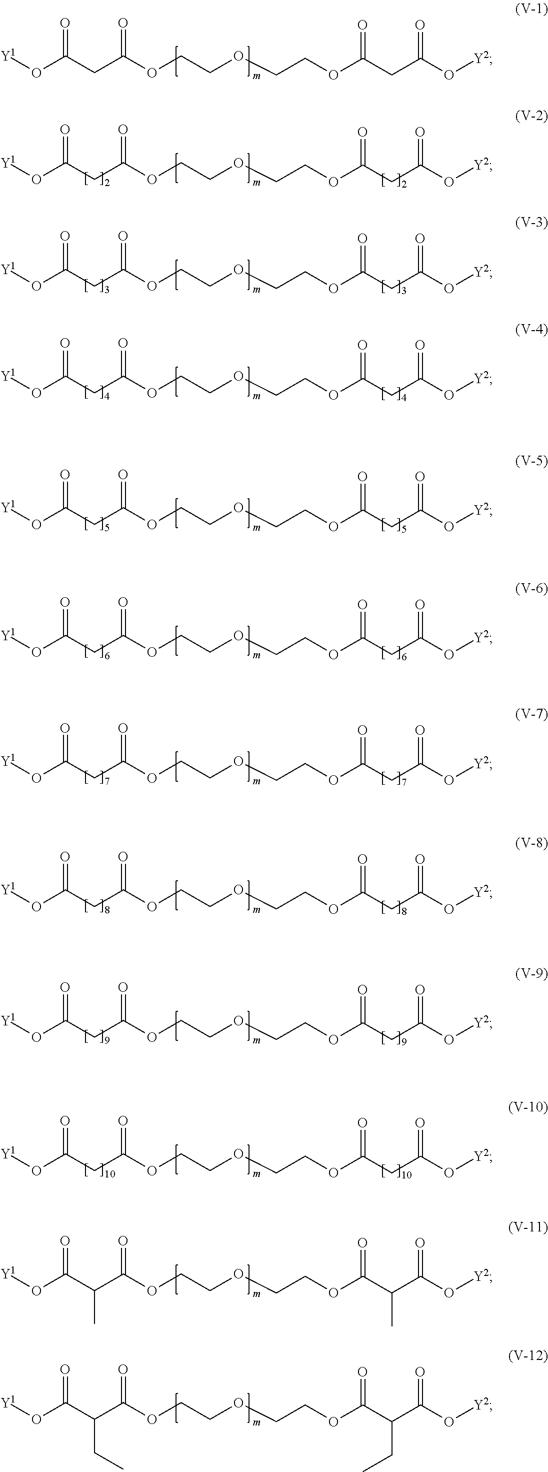 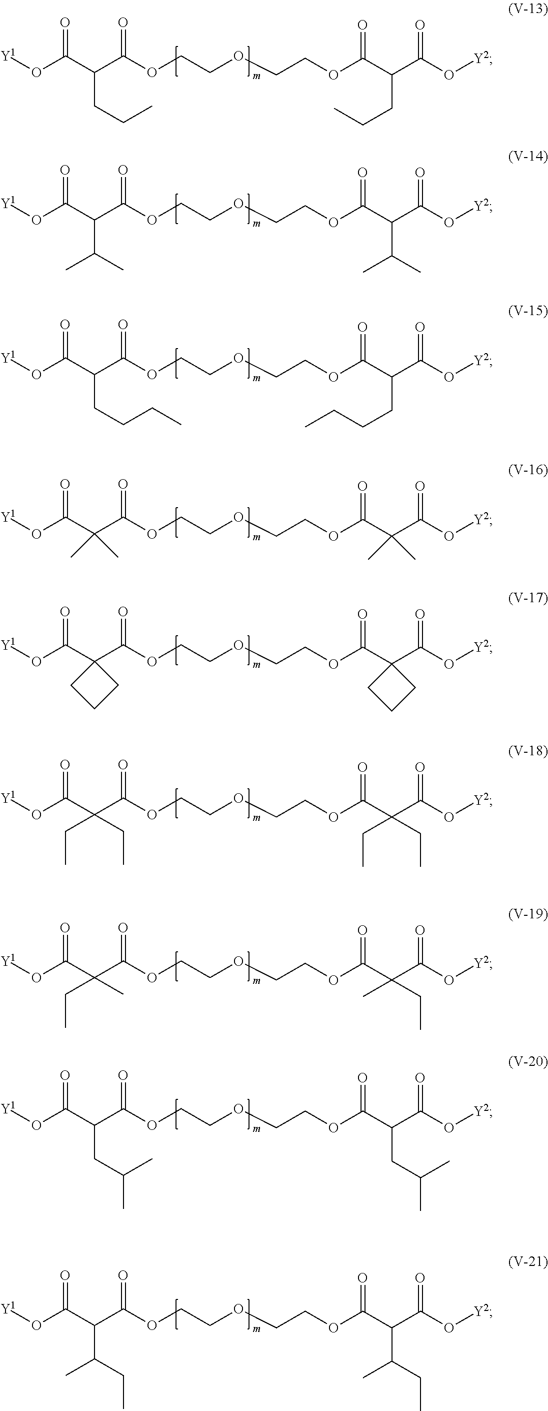 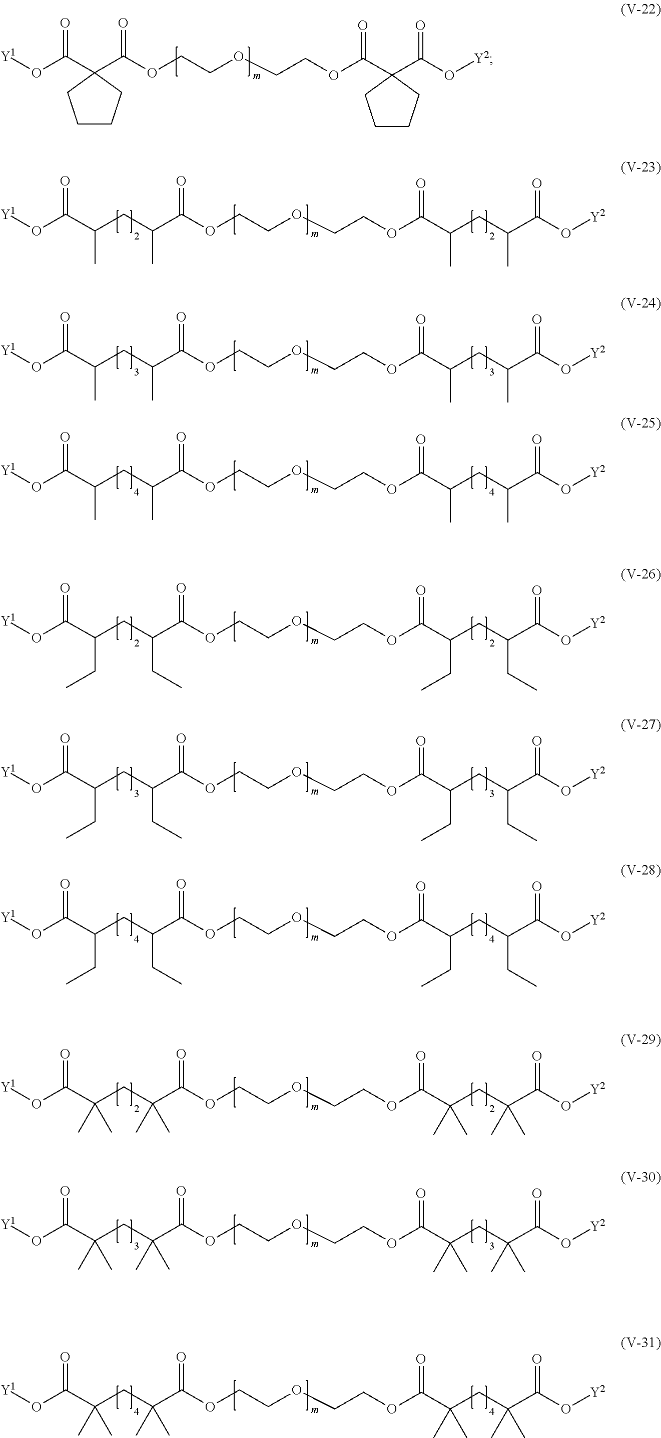 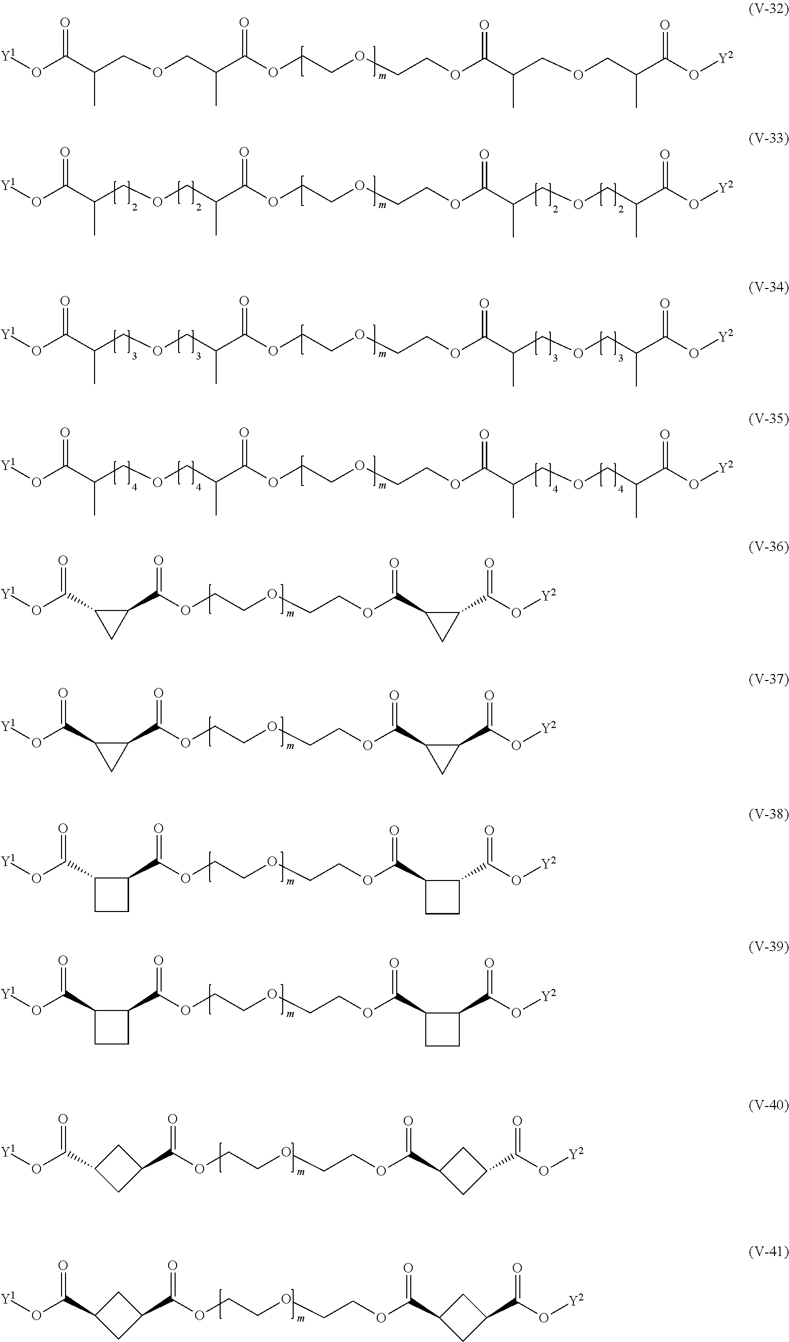 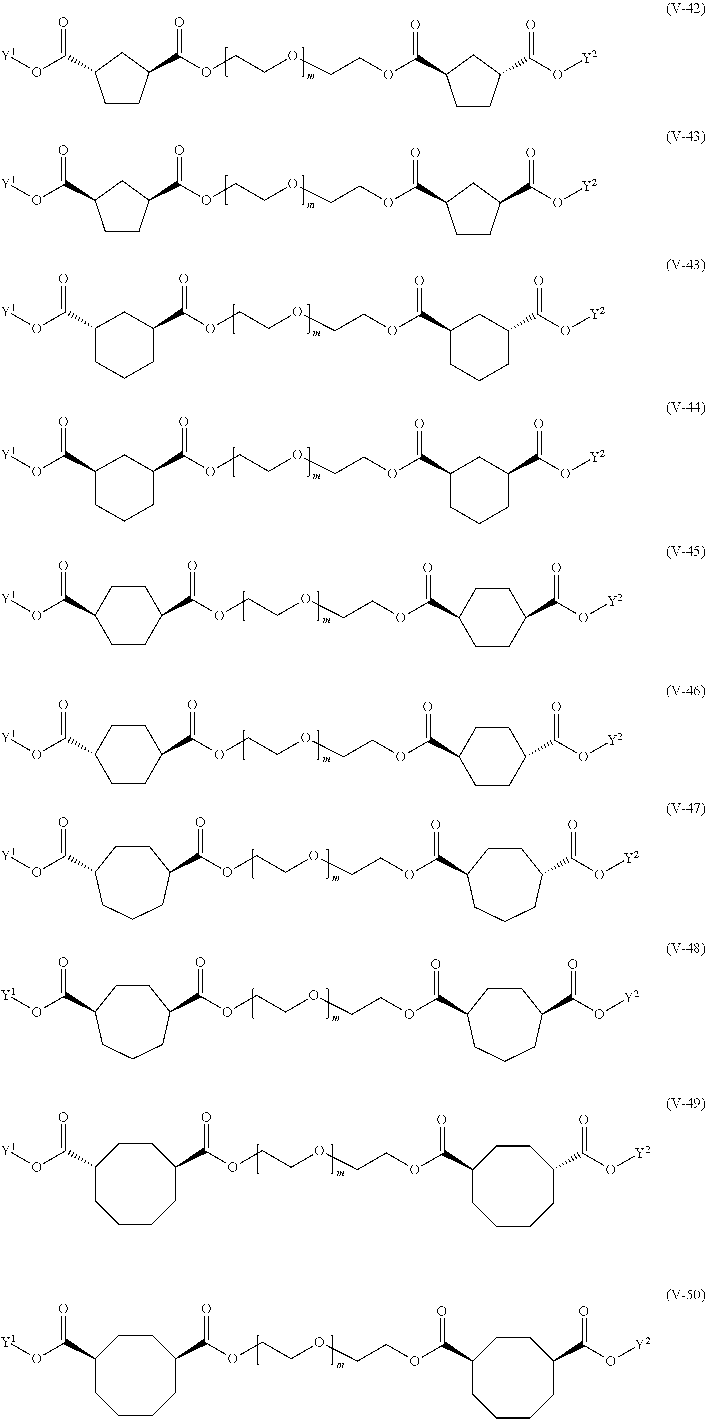 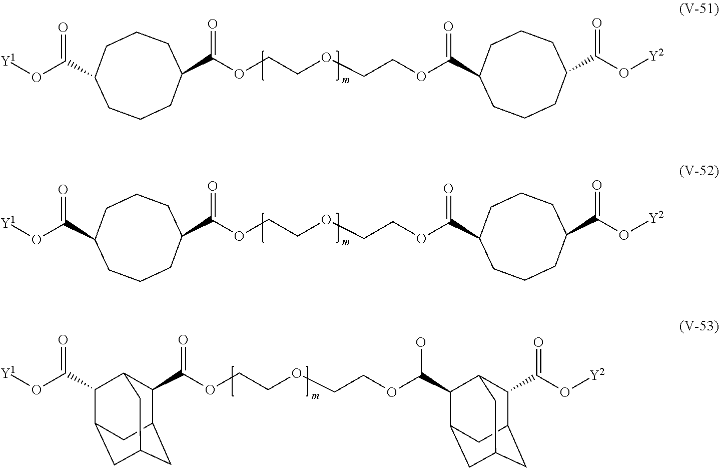
wherein:
each crosslinker reagent may be in the form of its racemic mixture, where applicable;
m ranges from 120 to 920; and
Y and Y² are identical or different and each is independently of the other selected from formulas (f-i) to (f-vi): 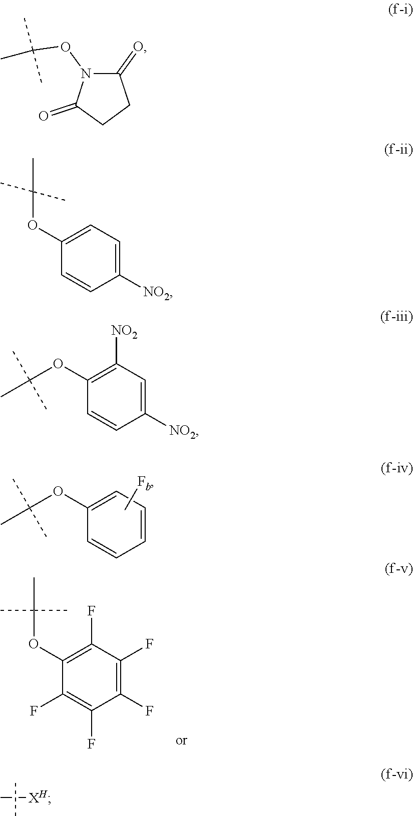
wherein
the dashed lines indicate attachment to the rest of the molecule;
b is 1, 2, 3, or 4 and
X^{H} is Cl, Br, J, or F.

28. The method of claim 1,
wherein the hydrogel-linked prodrug comprises a biologically active moiety selected from the group consisting of:
anesthetics and analgesics, antiallergenics, antihistamines, anti-inflammatory agents, anti-cancer agents, antibiotics, antiinfectives, antibacterials, anti-fungal agents, anti-viral agents, cell transport/mobility impending agents, antiglaucoma drugs, antihypertensives, decongestants, immunological response modifiers, immunosuppresive agents, peptides and proteins, steroidal compounds (steroids), low solubility steroids, carbonic anhydrize inhibitors, diagnostic agents, antiapoptosis agents, gene therapy agents, sequestering agents, reductants, antipermeability agents, antisense compounds, antiproliferative agents, antibodies and antibody conjugates, bloodflow enhancers, antiparasitic agents, non-steroidal anti inflammatory agents, nutrients and vitamins, enzyme inhibitors, antioxidants, anticataract drugs, aldose reductase inhibitors, cytoprotectants, cytokines, cytokine inhibitors, and cytokine protectants, UV blockers, and mast cell stabilizers; and
anti neovascular agents, including antiangiogenic agents, neuroprotectants, miotics and anti-cholinesterase, mydriatics, artificial tear and dry eye therapies, anti-TNFα, IL-1 receptor antagonists, protein kinase C-β inhibitors, somatostatin analogs, and sympathomimetics.
